SEQUENCE LISTING (76 sequences)

SEQ ID NO: 1 - protein, 9 aa, human herpesvirus 1
LIDGIFLRY

SEQ ID NO: 2 - protein, 9 aa, human herpesvirus 1
YMESVFQMY

SEQ ID NO: 3 - protein, 10 aa, human herpesvirus 1
HTDLHPNNTY

SEQ ID NO: 4 - protein, 9 aa, human herpesvirus 1
ATDSLNNEY

SEQ ID NO: 5 - protein, 9 aa, human herpesvirus 1
RSSLGSLLY

SEQ ID NO: 6 - protein, 9 aa, human herpesvirus 1
FTAPEVGTY

SEQ ID NO: 7 - protein, 10 aa, human herpesvirus 1
SALPTNADLY

SEQ ID NO: 8 - protein, 10 aa, human herpesvirus 1
FTDALGIDEY

SEQ ID NO: 9 - protein, 9 aa, human herpesvirus 1
ETDPVTFLY

SEQ ID NO: 10 - protein, 9 aa, human herpesvirus 1
TLLELVVSV

SEQ ID NO: 11 - protein, 9 aa, human herpesvirus 1
FLWEDQTLL

SEQ ID NO: 12 - protein, 9 aa, human herpesvirus 1
SVYPYDEFV

SEQ ID NO: 13 - protein, 9 aa, human herpesvirus 1
FLIAYQPLL

SEQ ID NO: 14 - protein, 9 aa, human herpesvirus 1
RILGVLVHL

SEQ ID NO: 15 - protein, 9 aa, human herpesvirus 1
ILIEGIFFA

SEQ ID NO: 16 - protein, 9 aa, human herpesvirus 1
FLADAVVRL

SEQ ID NO: 17 - protein, 9 aa, human herpesvirus 1
ALLDRDCRV

SEQ ID NO: 18 - protein, 9 aa, human herpesvirus 1
RLLGFADTV

SEQ ID NO: 19 - protein, 9 aa, human herpesvirus 1
VYTPSPYVF

SEQ ID NO: 20 - protein, 9 aa, human herpesvirus 1
EYQRLYATF

SEQ ID NO: 21 - protein, 10 aa, human herpesvirus 1
AYSLLFPAPF

SEQ ID NO: 22 - protein, 9 aa, human herpesvirus 1
AYLPRPVEF

SEQ ID NO: 23 - protein, 9 aa, human herpesvirus 1
AYVSVLYRW

SEQ ID NO: 24 - protein, 9 aa, human herpesvirus 1
KYFYCNSLF

SEQ ID NO: 25 - protein, 10 aa, human herpesvirus 1
LYPDAPPLRL

SEQ ID NO: 26 - protein, 10 aa, human herpesvirus 1
SSGVVFGTWY

SEQ ID NO: 27 - protein, 9 aa, human herpesvirus 1
AVLCLYLLY

SEQ ID NO: 28 - protein, 9 aa, human herpesvirus 1
YVAGFLALY

SEQ ID NO: 29 - protein, 9 aa, human herpesvirus 1
YLWIPASHY

SEQ ID NO: 30 - protein, 9 aa, human herpesvirus 1
VYMSPFYGY

SEQ ID NO: 31 - protein, 9 aa, human herpesvirus 1
FTFGGGYVY

SEQ ID NO: 32 - protein, 9 aa, human herpesvirus 1
ALLAKMLFY

SEQ ID NO: 33 - protein, 9 aa, human herpesvirus 1
YMANQILRY

SEQ ID NO: 34 - protein, 9 aa, human herpesvirus 1
LASDPHYEY

SEQ ID NO: 35 - protein, 9 aa, human herpesvirus 1
AILTQYWKY

SEQ ID NO: 36 - protein, 9 aa, human herpesvirus 1
LLAYVSVLY

SEQ ID NO: 37 - protein, 9 aa, human herpesvirus 1
SIVHHHAQY

SEQ ID NO: 38 - protein, 9 aa, human herpesvirus 1
ALATVTLKY

SEQ ID NO: 39 - protein, 9 aa, human herpesvirus 1
VPGWSRRTL

SEQ ID NO: 40 - protein, 9 aa, human herpesvirus 1
VPRPDDPVL

SEQ ID NO: 41 - protein, 10 aa, human herpesvirus 1
RPTERPRAPA

SEQ ID NO: 42 - protein, 9 aa, human herpesvirus 1
APRIGGRRA

SEQ ID NO: 43 - protein, 9 aa, human herpesvirus 1
VVRGPTVSL

SEQ ID NO: 44 - protein, 9 aa, human herpesvirus 1
CPRRPAVAF

SEQ ID NO: 45 - protein, 9 aa, human herpesvirus 1
APASVYQPA

SEQ ID NO: 46 - protein, 224 aa, human herpesvirus 1
MGILGWVGLIAVGILCVRGGLPSTEYVIRSRVAREVGDILKVPCVPLPSDDLDWRYETPSAINYALIDGIFLRYHCPGLDTVLWDRHAQRAYWVNPFLFGAGFLEDLSHPAFPADTQETETRLALYKEIRQALDSRKQAASHTPVKAGCVNFDYSRTRRCVGRQDLGLTNRTSGRTPVLPSDDEAGLQPKPLTTPSPIIATSDPTPRRDAATKSRRRRPHFRGL

SEQ ID NO: 47 - protein, 518 aa, human herpesvirus 1
MDESRRQRPAGHVAANLSPQGARQRSFKDWLASYVHSNPHGASGRPSGPSLQDAAVSRSSHGSRHRSGLRERLRAGLSRWRMSRSSHRRASPETPGTAAKLNRPPLRRSQAALTAPPSSPSHILTLTRIRKLCSPVFAINPALHYTTLEIPGARSFGGSGGYGDVQLIREHKLAVKTIKEKEWFAVELIATLLVGECVLRAGRTHNIRGFIAPLGFSLQQRQIVFPAYDMDLGKYIGQLASLRTTNPSVSTALHQCFTELARAVVFLNTTCGISHLDIKCANILVMLRSDAVSLRRAVLADFSLVTLNSNSTIARGQFCLQEPDLKSPRMFGMPTALTTANFHTLVGHGYNQPPELLVKYLNNERAEFTNHRLKHDVGLAVDLYALGQTLLELVVSVYVAPSLGVPVTRFPGYQYFNNQLSPDFALALLAYRCVLHPALFVNSAETNTHGLAYDVPEGIRRHLRNPKIRRAFTDRCINYQHTHKAILSSVALPPELKPLLVLVSRLCHTNPCARHALS

SEQ ID NO: 48 - protein, 535 aa, human herpesvirus 1
MELSYATTMHYRDVVFYVTTDRNRAYFVCGGCVYSVGRPCASQPGEIAKFGLVVRGTGPDDRVVANYVRSELRQRGLQDVRPIGEDEVFLDSVCLLNPNVSSELDVINTNDVEVLDECLAEYCTSLRTSPGVLISGLRVRAQDRIIELFEHPTIVNVSSHFVYTPSPYVFALAQAHLPRLPSSLEALVSGLFDGIPAPRQPLDAHNPRTDVVITGRRAPRPIAGSGAGSGGAGAKRATVSEFVQVKHIDRVGPAGVSPAPPPNNTDSSSLVPGAQDSAPPGPTLRELWWVFYAADRALEEPRADSGLTREEVRAVRGFREQAWKLFGSAGAPRAFIGAALGLSPLQKLAVYYYIIHRERRLSPFPALVRLVGRYTQRHGLYVPRPDDPVLADAINGLFRDALAAGTTAEQLLMFDLLPPKDVPVGSDVQADSTALLRFIESQRLAVPGGVISPEHVAYLGAFLSVLYAGRGRMSAATHTARLTGVTSLVLAVGDVDRLSAFDRGAAGAASRTRAAGYLDVLLTVRLARSQHGQSV

SEQ ID NO: 49 - protein, 580 aa, human herpesvirus 1
MDPYCPFDALDVWEHRRFIVADSRNFITPEFPRDFWMSPVFNLPRETAAEQVVVLQAQRTAAAAALENAAMQAAELPVDIERRLRPIERNVHEIAGALEALETAAAAAEEADAARGDEPAGGGDGGAPPGLAVAEMEVQIVRNDPPLRYDTNLPVDLLHMVYAGRGATGSSGVVFGTWYRTIQDRTITDFPLTTRSADFRDGRMSKTFMTALVLSLQACGRLYVGQRHYSAFECAVLCLYLLYRNTHGAADDSDRAPVTFGDLLGRLPRYLACLAAVIGTEGGRPQYRYRDDKLPKTQFAAGGGRYEHGALASHIVIATLMHHGVLPAAPGDVPRDASTHVNPDGVAHHDDINRAAAAFLSRGHNLFLWEDQTLLRATANTITALGVIQRLLANGNVYADRLNNRLQLGMLIPGAVPSEAIARGASGSDSGAIKSGDNNLEALCANYVLPLYRADPAVELTQLFPGLAALCLDAQAGRPVGSTRRVVDMSSGARQAALVRLTALELINRTRTNPTPVGEVIHAHDALAIQYEQGLGLLAQQARIGLGSNTKRFSAFNVSSDYDMLYFLCLGFIPQYLSAV

SEQ ID NO: 50 - protein, 635 aa, human herpesvirus 1
MAADAPGDRMEEPLPDRAVPIYVAGFLALYDSGDSGELALDPDTVRAALPPDNPLPINVDHRAGCEVGRVLAVVDDPRGPFFVGLIACVQLERVLETAASAAIFERRGPPLSREERLLYLITNYLPSVSLATKRLGGEAHPDRTLFAHVALCAIGRRLGTIVTYDTGLDAAIAPFRHLSPASREGARRLAAEAELALSGRTWAPGVEALTHTLLSTAVNNMMLRDRWSLVAERRRQAGIAGHTYLQASEKFKMWGAEPVSAPARGYKNGAPESTDIPPGSIAAAPQGDRCPIVRQRGVALSPVLPPMNPVPTSGTPAPAPPGDGSYLWIPASHYNQLVAGHAAPQPQPHSAFGFPAAAGSVAYGPHGAGLSQHYPPHVAHQYPGVLFSGPSPLEAQIAALVGAIAADRQAGGQPAAGDPGVRGSGKRRRYEAGPSESYCDQDEPDADYPYYPGEARGAPRGVDSRRAARHSPGTNETITALMGAVTSLQQELAHMRARTSAPYGMYTPVAHYRPQVGEPEPTTTHPALCPPEAVYRPPPHSAPYGPPQGPASHAPTPPYAPAACPPGPPPPPCPSTQTRAPLPTEPAFPPAATGSQPEASNAEAGALVNASSAAHVDVDTARAADLFVSQMMGAR

SEQ ID NO: 51 - protein, 904 aa, human herpesvirus 1
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 52 - protein, 1196 aa, human herpesvirus 1
METKPKTATTIKVPPGPLGYVYARACPSEGIELLALLSARSGDSDVAVAPLVVGLTVESGFEANVAVVVGSRTTGLGGTAVSLKLTPSHYSSSVYVFHGGRHLDPSTQAPNLTRLCERARRHFGFSDYTPRPGDLKHETTGEALCERLGLDPDRALLYLVVTEGFKEAVCINNTFLHLGGSDKVTIGGAEVHRIPVYPLQLFMPDFSRVIAEPFNANHRSIGEKFTYPLPFFNRPLNRLLFEAVVGPAAVALRCRNVDAVARAAAHLAFDENHEGAALPADITFTAFEASQGKTPRGGRDGGGKGAAGGFEQRLASVMAGDAALALESIVSMAVFDEPPTDISAWPLFEGQDTAAARANAVGAYLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGAVIVGRQEMDVFRYVADSNQTDVPCNLCTFDTRHACVHTTLMRLRARHPKFASAARGAIGVFGTMNSMYSDCDVLGNYAAFSALKRADGSETARTIMQETYRAATERVMAELETLQYVDQAVPTAMGRLETIITNREALHTVVNNVRQVVDREVEQLMRNLVEGRNFKFRDGLGEANHAMSLTLDPYACGPCPLLQLLGRRSNLAVYQDLALSQCHGVFAGQSVEGRNFRNQFQPVLRRRVMDMFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAAIFPNGKPPGSNQPNPQWFWTALQRNQLPARLLSREDIETIAFIKKFSLDYGAINFINLAPNNVSELAMYYMANQILRYCDHSTYFINTLTAIIAGSRRPPSVQAAAAWSAQGGAGLEAGARALMDAVDAHPGAWTSMFASCNLLRPVMAARPMVVLGLSISKYYGMAGNDRVFQAGNWASLMGGKNACPLLIFDRTRKFVLACPRAGFVCAASSLGGGAHESSLCEQLRGIISEGGAAVASSVFVATVKSLGPRTQQLQIEDWLALLEDEYLSEEMMELTARALERGNGEWSTDAALEVAHEAEALVSQLGNAGEVFNFGDFGCEDDNATPFGGPGAPGPAFAGRKRAFHGDDPFGEGPPDKKGDLTLDML

SEQ ID NO: 53 - protein, 306 aa, human herpesvirus 1
MYDTDPHRRGSRPGPYHGKERRRSRSSAAGGTLGVVRRASRKSLPPHARKQELCLHERQRYRGLFAALAQTPSEEIAIVRSLSVPLVKTTPVSLPFCLDQTVADNCLTLSGMGYYLGIGGCCPACNAGDGRFAATSREALILAFVQQINTIFEHRAFLASLVVLADRHNAPLQDLLAGILGQPELFFVHTILRGGGACDPRLLFYPDPTYGGHMLYVIFPGTSAHLHYRLIDRMLTACPGYRFVAHVWQSTFVLVVRRNAEKPTDAEIPTVSAADIYCKMRDISFDGGLMLEYQRLYATFDEFPPP

SEQ ID NO: 54 - protein, 1123 aa, human herpesvirus 1
MADRGLPSEAPVVTTSPAGPPSDGPMQRLLASLAGLRQPPTPTAETANGADDPAFLATAKLRAAMAAFLLSGTAIAPADARDCWRPLLEHLCALHRAHGLPETALLAENLPGLLVHRLVVALPEAPDQAFREMEVIKDTILAVTGSDTSHALDSAGLRTAAALGPVRVRQCAVEWIDRWQTVTKSCLAMSPRTSIEALGETSLKMAPVPLGQPSANLTTPAYSLLFPAPFVQEGLRFLALVSNRVTLFSAHLQRIDDATLTPLTRALFTLALVDEYLTTPERGAVVPPPLLAQFQHTVREIDPAIMIPPLEANKMVRSREEVRVSTALSRVSPRSACAPPGTLMARVRTDVAVFDPDVPFLSSSALAVFQPAVSSLLQLGEQPSAGAQQRLLALLQQTWTLIQNTNSPSVVINTLIDAGFTPSHCTHYLSALEGFLAAGVPARTPTGHGLGEVQQLFGCIALAGSNVFGLAREYGYYANYVKTFRRVQGASEHTHGRLCEAVGLSGGVLSQTLARIMGPAVPTEHLASLRRALVGEFETAERRFSSGQPSLLRETALIWIDVYGQTHWDITPTTPATPLSALLPVGQPSHAPSVHLAAATQIRFPALEGIHPNVLADPGFVPYVLALVVGDALRATCSAAYLPRPVEFALRVLAWARDFGLGYLPTVEGHRTKLGALITLLEPAARGGLGPTMQMADNIEQLLRELYVISRGAVEQLRPLVQLQPPPPPEVGTSLLLISMYALAARGVLQDLAERADPLIRQLEDAIVLLRLHMRTLSAFFECRFESDGRRLYAVVGDTPDRLGPWPPEAMGDAVSQYCSMYHDAKRALVASLASLRSVITETTAHLGVCDELAAQVSHEDNVLAVVRREIHGFLSVVSGIHARASKLLSGDQVPGFCFMGQFLARWRRLSACYQAARAAAGPEPVAEFVQELHDTWKGLQTERAVVVAPLVSSADQRAAAIREVMAHAPEDAPPQSPAADRVVLTSRRDLGAWGDYSLGPLGQTTAVPDSVDLSRQGLAVTLSMDWLLMNELLRVTDGVFRASAFRPLAGPESPRDLEVRDAGNSLPAPMPMDAQKPEAYGHGPRQADREGAPHSNTPVEDDEMIPEDTVAPPTDLPLTSYQ

SEQ ID NO: 55 - protein, 1137 aa, human herpesvirus 1
MASRPAASSPVEARAPVGGQEAGGPSAATQGEAAGAPLAHGHHVYCQRVNGVMVLSDKTPGSASYRISDNNFVQCGSNCTMIIDGDVVRGRPQDPGAAASPAPFVAVTNIGAGSDGGTAVVAFGGTPRRSAGTSTGTQTADVPTEALGGPPPPPRFTLGGGCCSCRDTRRRSAVFGGEGDPVGPAEFVSDDRSSDSDSDDSEDTDSETLSHASSDVSGGATYDDALDSDSSSDDSLQIDGPVCRPWSNDTAPLDVCPGTPGPGADAGGPSAVDPHAPTPEAGAGLAADPAVARDDAEGLSDPRPRLGTGTAYPVPLELTPENAEAVARFLGDAVNREPALMLEYFCRCAREETKRVPPRTFGSPPRLTEDDFGLLNYALVEMQRLCLDVPPVPPNAYMPYYLREYVTRLVNGFKPLVSRSARLYRILGVLVHLRIRTREASFEEWLRSKEVALDFGLTERLREHEAQLVILAQALDHYDCLIHSTPHTLVERGLQSALKYEEFYLKRFGGHYMESVFQMYTRIAGFLACRATRGMRHIALGREGSWWEMFKFFFHRLYDHQIVPSTPAMLNLGTRNYYTSSCYLVNPQATTNKATLRAITSNVSAILARNGGIGLCVQAFNDSGPGTASVMPALKVLDSLVAAHNKESARPTGACVYLEPWHTDVRAVLRMKGVLAGEEAQRCDNIFSALWMPDLFFKRLIRHLDGEKNVTWTLFDRDTSMSLADFHGEEFEKLYQHLEVMGFGEQIPIQELAYGIVRSAATTGSPFVMFKDAVNRHYIYDTQGAAIAGSNLCTEIVHPASKRSSGVCNLGSVNLARCVSRQTFDFGRLRDAVQACVLMVNIMIDSTLQPTPQCTRGNDNLRSMGIGMQGLHTACLKLGLDLESAEFQDLNKHIAEVMLLSAMKTSNALCVRGARPFNHFKRSMYRAGRFHWERFPDARPRYEGEWEMLRQSMMKHGLRNSQFVALMPTAASAQISDVSEGFAPLFTNLFSKVTRDGETLRPNTLLLKELERTFSGKRLLEVMDSLDAKQWSVAQALPCLEPTHPLRRFKTAFDYDQKLLIDLCADRAPYVDHSQSMTLYVTEKADGTLPASTLVRLLVHAYKRGLKTGMYYCKVRKATNSGVFGGDDNIVCMSCAL

SEQ ID NO: 56 - protein, 340 aa, human herpesvirus 1
MDSAAPALSPALTALTDQSATADLAIQIPKCPDPERYFYTSQCPDINHLRSLSILNRWLETELVFVGDEEDVSKLSEGELSFYRFLFAFLSAADDLVTENLGGLSGLFEQKDILHYYVEQECIEVVHSRVYNIIQLVLFHNNDQARREYVAGTINHPAIRAKVDWLEARVRECASVPEKFILMILIEGIFFAASFAAIAYLRTNNLLRVTCQSNDLISRDEAVHTTASCYIYNNYLGGHAKPPPDRVYGLFRQAVEIEIGFIRSQAPTDSHILSPAALAAIENYVRFSADRLLGLIHMKPLFSAPPPDASFPLSLMSTDKHTNFFECRSTSYAGAVVNDL

SEQ ID NO: 57 - protein, 489 aa, human herpesvirus 1
MGLFGMMKFAHTHHLVKRRGLGAPAGYFTPIAVDLWNVMYTLVVKYQRRYPSYDREAITLHCLCRLLKVFTQKSLFPIFVTDRGVNCMEPVVFGAKAILARTTAQCRTDEEASDVDASPPPSPITDSRPSSAFSNMRRRGTSLASGTRGTAGSGAALPSAAPSKPALRLAHLFCIRVLRALGYAYINSGQLEADDACANLYHTNTVAYVYTTDTDLLLMGCDIVLDISACYIPTINCRDILKYFKMSYPQFLALFVRCHTDLHPNNTYASVEDVLRECHWTPPSRSQTRRAIRREHTSSRSTETRPPLPPAAGGTETRVSWTEILTQQIAGGYEDDEDLPLDPRDVTGGHPGPRSSSSEILTPPELVQVPNAQLLEEHRSYVANPRRHVIHDAPESLDWLPDPMTITELVEHRYIKYVISLIGPKERGPWTLLKRLPIYQDIRDENLARSIVTRHITAPDIADRFLEQLRTQAPPPAFYKDVLAKFWDE

SEQ ID NO: 58 - protein, 718 aa, human herpesvirus 1
MQRRTRGASSLRLARCLTPANLIRGDNAGVPERRIFGGCLLPTPEGLLSAAVGALRQRSDDAQPAFLTCTDRSVRLAARQHNTVPESLIVDGLASDPHYEYIRHYASAATQALGEVELPGGQLSRAILTQYWKYLQTVVPSGLDVPEDPVGDCDPSLHVLLRPTLAPKLLARTPFKSGAVAAKYAATVAGLRDALHRIQQYMFFMRPADPSRPSTDTALRLNELLAYVSVLYRWASWMLWTTDKHVCHRLSPSNRRFLPLGGSPEAPAETFARHLDRGPSGTTGSMQCMALRAAVSDVLGHLTRLANLWQTGKRSGGTYGTVDTVVSTVEVLSIVHHHAQYIINATLTGYGVWATDSLNNEYLRAAVDSQERFCRTTAPLFPTMTAPSWARMELSIKAWFGAALAADLLRNGAPSLHYESILRLVASRRTTWSAGPPPDDMASGPGGHRAGGGTCREKIQRARRDNEPPPLPRPRLHSTPASTRRFRRRRADGAGPPLPDANDPVAEPPAAATQPATYYTHMGEVPPRLPARNVAGPDRRPPAATCPLLVRRASLGSLDRPRVWGPAPEGEPDQMEATYLTADDDDDDARRKATHAASARERHAPYEDDESIYETVSEDGGRVYEEIPWMRVYENVCVNTANAAPASPYIEAENPLYDWGGSALFSPPGRTGPPPPPLSPSPVLARHRANALTNDGPTNVAALSALLTKLKREGRRSR

SEQ ID NO: 59 - protein, 693 aa, human herpesvirus 1
MSAREPAGRRRRASTRPRASPVADEPAGDGVGFMGYLRAVFRGDDDSELEALEEMAGDEPPVRRRREGPRARRRRASEAPPTSHRRASRQRPGPDAARSQSVRGRLDDDDEVPRGPPQARQGGYLGPVDARAILGRVGGSRVAPSPLFLEELQYEEDDYPEAVGPEDGGGARSPPKVEVLEGRVPGPELRAAFPLDRLAPQVAVWDESVRSALALGHPAGFYPCPDSAFGLSRVGVMHFASPDNPAVFFRQTLQQGEALAWYITGDGILDLTDRRTKTSPAQAMSFLADAVVRLAINGWVCGTRLHAEARGSDLDDRAAELRRQFASLTALRPVGAAAVPLLSAGGLVSPQSGPDAAVFRSSLGSLLYWPGVRALLDRDCRVAARYAGRMTYLATGALLARFNPDAVRCVLTREAAFLGRVLDVLAVMAEQTVQWLSVVVGARLHPHVHHPAFADVAREELFRALPLGSPAVVGAEHEALGDTAARRLLANSGLNAVLGAAVYALHTALATVTLKYARACGDAHRRRDDAAATRAILAAGLVLQRLLGFADTVVACVTLAAFDGGFTAPEVGTYTPLRYACVLRATQPLYARTTPAKFWADVRAAAEHVDLRPASSAPRAPVSGTADPAFLLKDLEPFPPAPVSGGSVLGPRVRVVDIMSQFRKLLMGDEGAAALRAHVSGRRATGLGGPPRP

SEQ ID NO: 60 - protein, 490 aa, human herpesvirus 1
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 61 - protein, 301 aa, human herpesvirus 1
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 62 - protein, 338 aa, human herpesvirus 1
MLAVRSLQHLSTVVLITAYGLVLVWYTVFGASPLHRCIYAVRPTGTNNDTALVWMKMNQTLLFLGAPTHPPNGGWRNHAHICYANLIAGRVVPFQVPPDAMNRRIMNVHEAVNCLETLWYTRVRLVVVGWFLYLAFVALHQRRCMFGVVSPAHKMVAPATYLLNYAGRIVSSVFLQYPYTKITRLLCELSVQRQNLVQLFETDPVTFLYHRPAIGVIVGCELMLRFVAVGLIVGTAFISRGACAITYPLFLTITTWCFVSTIGLTELYCILRRGPAPKNADKAAAPGRSKGLSGVCGRCCSIILSGIAVRLCYIAVVAGVVLVALHYEQEIQRRLFDV

SEQ ID NO: 63 - protein, 512 aa, human herpesvirus 1
MATDIDMLIDLGLDLSDSDLDEDPPEPAESRRDDLESDSSGECSSSDEDMEDPHGEDGPEPILDAARPAVRPSRPEDPGVPSTQTPRPTERQGPNDPQPAPHSVWSRLGARRPSCSPEQHGGKVARLQPPPTKAQPARGGRRGRRRGRGRGGPGAADGLSDPRRRAPRTNRNPGGPRPGAGWTDGPGAPHGEAWRGSEQPDPPGGQRTRGVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTTAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 64 - protein, 420 aa, human herpesvirus 1
MADISPGAFAPCVKARRPALRSPPLGTRKRKRPSRPLSSESEVESDTALESEVESETASDSTESGDQDEAPRIGGRRAPRRLGGRFFLDMSAESTTGTETDASVSDDPDDTSDWSYDDIPPRPKRARVNLRLTSSPDRRDGVIFPKMGRVRSTRETQPRAPTPSAPSPNAMLRRSVRQAQRRSSARWTPDLGYMRQCINQLFRVLRVARDPHGSANRLRHLIRDCYLMGYCRARLAPRTWCRLLQVSGGTWGMHLRNTIREVEARFDATAEPVCKLPCLETRRYGPECDLSNLEIHLSATSDDEISDATDLEAAGSDHTLASQSDTEDAPSPVTLETPEPRGSLAVRLEDEFGEFDWTPQEGSQPWLSAVVADTSSVERPGPSDSGAGRAAEDRKCLDGCRKMRFSTACPYPCSDTFLRP

SEQ ID NO: 65 - protein, 390 aa, human herpesvirus 1
MPCRPLQGLVLVGLWVCATSLVVRGPTVSLVSNSFVDAGALGPDGVVEEDLLILGELRFVGDQVPHTTYYDGGVELWHYPMGHKCPRVVHVVTVTACPRRPAVAFALCRATDSTHSPAYPTLELNLAQQPLLRVQRATRDYAGVYVLRVWVGDAPNASLFVLGMAIAAEGTLAYNGSAYGSCDPKLLPSSAPRLAPASVYQPAPNQASTPSTTTSTPSTTIPAPSTTIPAPQASTTPFPTGDPKPQPPGVNHEPPSNATRATRDSRYALTVTQIIQIAIPASIIALVFLGSCICFIHRCQRRYRRSRRPIYSPQMPTGISCAVNEAAMARLGAELKSHPSTPPKSRRRSSRTPMPSLTAIAEESEPAGAAGLPTPPVDPTTPTPTPPLLV

SEQ ID NO: 66 - protein, 775 aa, human herpesvirus 1
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAAPRGASGPSGGVGVGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPLPSAASVEQEAAVRPRKRRGSGQENPSPQSTRPPLAPAGAKRAATHPPSDSGPGGRGQGGPGTPLTSSAASASSSSASSSSAPTPAGAASSAAGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVMPPEYPTAPASEWNSLWMTPVGNMLFDQGTLVGALDFRSLRSRHPWSGEQGASTRDEGKQ

SEQ ID NO: 67 - protein, 1298 aa, human herpesvirus 1
MASENKQRPGSPGPTDGPPPTPSPDRDERGALGWGAETEEGGDDPDHDPDHPHDLDDARRDGRAPAAGTDAGEDAGDAVSPRQLALLASMVEEAVRTIPTPDPAASPPRTPAFRADDDDGDEYDDAADAAGDRAPARGREREAPLRGAYPDPTDRLSPRPPAQPPRRRRHGRWRPSASSTSSDSGSSSSSSASSSSSSSDEDEDDDGNDAADHAREARAVGRGPSSAAPAAPGRTPPPPGPPPLSEAAPKPRAAARTPAASAGRIERRRARAAVAGRDATGRFTAGQPRRVELDADATSGAFYARYRDGYVSGEPWPGAGPPPPGRVLYGGLGDSRPGLWGAPEAEEARRRFEASGAPAAVWAPELGDAAQQYALITRLLYTPDAEAMGWLQNPRVVPGDVALDQACFRISGAARNSSSFITGSVARAVPHLGYAMAAGRFGWGLAHAAAAVAMSRRYDRAQKGFLLTSLRRAYAPLLARENAALTGAAGSPGAGADDEGVAAVAAAAPGERAVPAGYGAAGILAALGRLSAAPASPAGGDDPDAARHADADDDAGRRAQAGRVAVECLAACRGILEALAEGFDGDLAAVPGLAGARPASPPRPEGPAGPASPPPPHADAPRLRAWLRELRFVRDALVLMRLRGDLRVAGGSEAAVAAVRAVSLVAGALGPALPRDPRLPSSAAAAAADLLFDNQSLRPLLAAAASAPDAADALAAAAASAAPREGRKRKSPGPARPPGGGGPRPPKTKKSGADAPGSDARAPLPAPAPPSTPPGPEPAPAQPAAPRAAAAQARPRPVAVSRRPAEGPDPLGGWRRQPPGPSHTAAPAAAALEAYCSPRAVAELTDHPLFPVPWRPALMFDPRALASIAARCAGPAPAAQAACGGGDDDDNPHPHGAAGGRLFGPLRASGPLRRMAAWMRQIPDPEDVRVVVLYSPLPGEDLAGGGASGGPPEWSAERGGLSCLLAALANRLCGPDTAAWAGNWTGAPDVSALGAQGVLLLSTRDLAFAGAVEFLGLLASAGDRRLIVVNTVRACDWPADGPAVSRQHAYLACELLPAVQCAVRWPAARDLRRTVLASGRVFGPGVFARVEAAHARLYPDAPPLRLCRGGNVRYRVRTRFGPDTPVPMSPREYRRAVLPALDGRAAASGTTDAMAPGAPDFCEEEAHSHAACARWGLGAPLRPVYVALGREAVRAGPARWRGPRRDFCARALLEPDDDAPPLVLRGDDDGPGALPPAPPGIRWASATGRSGTVLAAAGAVEVLGAEAGLATPPRREVVDWEGAWDEDDGGAFEGDGVL

SEQ ID NO: 68 - protein, 41 aa, artificial sequence
  synthetic peptide
MSGSHHHHHHSSGIEGRGRLIKHMTMASRLESTSLYKKAGF

SEQ ID NO: 69 - DNA, 32 bp, artificial sequence
  primer
ccgccgctagcatggccgtcatggcgccccga

SEQ ID NO: 70 - DNA, 30 bp, artificial sequence
  primer
ccgccctcgagtcacactttacaagctgtg

SEQ ID NO: 71 - DNA, 36 bp, artificial sequence
  primer
ccgccgctagcatgcgggtcacggcgccccgaaccg

SEQ ID NO: 72 - DNA, 34 bp, artificial sequence
  primer
ccgccctcgagtcaagctgtgagagacacatcag

SEQ ID NO: 73 - DNA, 34 bp, artificial sequence
  primer
ccgcctgctagcatgcgggtcatggcgccccgag

SEQ ID NO: 74 - DNA, 39 bp, artificial sequence
  primer
ccgcccgtctcgagtcaggctttacaagtgatgagagac

SEQ ID NO: 75 - DNA, 29 bp, artificial sequence
  primer
tataaagcttttctccccagacgccgaga

SEQ ID NO: 76 - DNA, 32 bp, artificial sequence
  primer
atatgcggccgcgtctcaggctttacaagcga

(19) United States
(12) Patent Application Publication
(10) Pub. No.: US 2013/0224236 A1
(43) Pub. Date: Aug. 29, 2013

(54) HSV-1 EPITOPES AND METHODS FOR USING SAME

(71) Applicants: David M. Koelle, Seattle, WA (US); Lichen Jing, Seattle, WA (US)

(72) Inventors: David M. Koelle, Seattle, WA (US); Lichen Jing, Seattle, WA (US)

(73) Assignee: UNIVERSITY OF WASHINGTON, Seattle, WA (US)

(21) Appl. No.: 13/882,405

(22) PCT Filed: Nov. 3, 2011
(86) PCT No.: PCT/US2011/059214

§ 371 Date: Apr. 29, 2013

Related U.S. Application Data
(60) Provisional application No. 61/409,683, filed on Nov. 3, 2010.
(60) Provisional application No. 61/475,808, filed on Apr. 15, 2011.

(51) Int. Cl. C07K 14/035

(52) U.S. Cl. CPC C07K 14/035
USPC 424/186.1; 530/350; 536/23.72; 435/320.1; 435/69.3; 435/235.1; 435/375; 435/252.3

(57) ABSTRACT

The invention provides HSV antigens and epitopes that are useful for the prevention and treatment of HSV infection. T-cells having specificity for antigens of the invention have demonstrated cytotoxic activity against cells loaded with virally-encoded peptide epitopes, and in many cases, against cells infected with HSV. The identification of immunogenic antigens responsible for T-cell specificity provides improved anti-viral therapeutic and prophylactic strategies. Compositions containing antigens or polynucleotides encoding antigens of the invention provide effectively targeted vaccines for prevention and treatment of HSV infection.

This application claims the benefit of U.S. provisional patent applications 61/409,683, filed Nov. 3, 2010, and 61/475,808, filed Apr. 15, 2011, the entire contents of each of which is incorporated herein by reference.

### STATEMENT REGARDING FEDERALLY SPONSORED RESEARCH

This invention was made with government support under grant number AI50132 and 1 R21 081060 awarded by the National Institutes of Health. The government has certain rights in the invention.

### TECHNICAL FIELD OF THE INVENTION

The invention relates to molecules, compositions and methods that can be used for the treatment and prevention of viral infection and other diseases. More particularly, the invention identifies epitopes of herpes simplex virus type 1 (HSV-1) proteins that can be used for methods involving molecules and compositions having the antigenic specificity of HSV-specific T cells. In addition, the invention relates to methods for detecting, treating and preventing HSV infection, as well as methods for inducing an immune response to HSV. The epitopes described herein are also useful in the development of diagnostic and therapeutic agents for detecting, preventing and treating viral infection and other diseases.

### BACKGROUND OF THE INVENTION

Herpes simplex type 1 (HSV-1) infects about 60% of people in the United States. Most people have either no symptoms or bothersome recurrent sores on the lips or face. Medically serious consequences of HSV-1 include herpes simplex encephalitis (HSE). HSE is usually a recurrence of HSV-1, and occurs in otherwise healthy, immunocompetent people. HSE can be fatal, and typically results in long term brain damage. Herpes simplex keratitis (HSK) is another serious consequence. HSK is part of a spectrum of HSV eye diseases that consume considerable health care resources; HSK can lead to blindness and a need for corneal transplantation. These and other complications are rare on a per-patient basis, but given the high prevalence of HSV-1, overall have a significant health care impact.

There is no HSV-1 vaccine. Vaccines for HSV that have been tested thus far have failed in clinical trials, including a recent phase III trial of an adjuvanted glycoprotein D (gD2) product (2). This vaccine elicits antibody and CD4 T-cell responses, but fails to induce CD8 responses. Newer platforms can elicit CD8 and CD4 cells, but they require rationally selected T-cell antigens. There is thus a need for new methods to permit measurement of both CD8 and CD4 responses to the complete HSV-1 proteome to begin rational prioritization of next-generation vaccine candidates.

Several recent observations support the concept that an effective HSV vaccine will need to induce coordinated CD8 and CD4 T-cell responses. HSV-1-specific CD8 T-cells localize to the site of HSV-1-infection in human and murine trigeminal ganglia (TG) (3-5) and both HSV-specific CD8 and CD4 T-cells localize to acute and healed sites of skin infection in mice and humans, suggesting that optimally programmed memory cells could monitor for infection or reactivation (6-8). In animals, HSV ganglionic load correlates with reactivation frequency, so pre-equipping a person with HSV-specific CD8 T-cells could reduce seeding of the ganglia, even if a primary infection occurs in recipients of a non-sterilizing vaccine, and ameliorate the chronic phase (9, 10). Strong CD8 responses can be protective against HSV infection specific mouse models (11). In murine protection models based on attenuated live virus or DNA vaccines, protection is more typically CD4-dependent, and in humans, HSV disease worsens with CD4 depletion in untreated human immunodeficiency virus type 1 (HIV-1) infection (12, 13).

The breadth and specificity of HSV-1-specific T-cells in humans is largely unknown. The virus has a large 152 kb genome encoding about 77 polypeptides (14, 15). A limited number of CD8 epitopes discovered in the context of HSV-2 research are sequence-identical and thus cross-reactive with HSV-1. In HSV-1-infected human eyes, CD4 reactivity has been demonstrated with proteins in the viral tegument encoded by genes UL21, UL46, UL47, and UL49 (18-23). Envelope glycoproteins gD1 and gB1 are also known CD4 antigens (24).

Thus rules governing CD8 specificity are an important issue for HSV vaccine design. HSV genes are expressed in sequential, coordinated kinetic waves during the viral replication cycle, and a subset of proteins are present in virions and injected into cells upon viral entry. Some replication-incompetent whole HSV vaccines are blocked at the DNA replication step, such that true-late proteins, which are made only after DNA replication, are not expressed (25). Other strains have a later replication block, with true-late proteins being synthesized in the cytoplasm of infected cells (26). This property is shared by attenuated but replication-competent candidates (27). There remains a need, therefore, to determine if the CD8 response is weighted towards any specific kinetic or structural subset of HSV-1 proteins.

There remains a need, however, to identify epitopes that can be used for effective vaccines for treatment and/or prevention of HSV infection.

### SUMMARY OF THE INVENTION

The invention provides HSV antigens, polypeptides comprising HSV antigens, polynucleotides encoding the polypeptides, vectors, and recombinant viruses containing the polynucleotides, antigen-presenting cells (APCs) presenting the polypeptides, immune cells directed against HSV, and pharmaceutical compositions. Compositions comprising these polypeptides, polynucleotides, viruses, APCs and immune cells can be used as vaccines. In particular, the invention provides HSV-1 antigens. In some embodiments, the antigens are specific to HSV-1 as compared to HSV-2. The pharmaceutical compositions can be used both prophylactically and therapeutically. The invention additionally provides methods, including methods for preventing and treating HSV infection, for killing HSV-infected cells, for inhibiting viral replication, for enhancing secretion of antiviral and/or immunomodulatory lymphokines, and for enhancing production of HSV-specific antibody. For preventing and treating HSV infection, for enhancing secretion of antiviral and/or immunomodulatory lymphokines, for enhancing production of HSV-specific antibody, and generally for stimulating and/or augmenting HSV-specific immunity, the method comprises administering to a subject a polypeptide, polynucleotide, recombinant virus, APC, immune cell or composition of the invention. The methods for killing HSV-infected cells and for inhibiting viral replication comprise contacting an HSV-infected cell with an immune cell of the invention. The immune cell of the invention is one that has been stimulated by an antigen of the invention or by an APC that presents an antigen of the invention. One format for presenting an antigen of the invention makes use of replication-competent or replication-incompetent, or appropriately killed, whole virus, such as HSV, that has been engineered to present one or more antigens of the invention. A method for producing immune cells of the invention is also provided. The method comprises contacting an immune cell with an APC, preferably a dendritic cell, that has been modified to present an antigen of the invention. In a preferred embodiment, the immune cell is a T cell such as a CD4+ or CD8+ T cell.

Specific HSV antigens and epitopes that have been identified by the method of the invention include those listed in Table 4 provided in Example 1 below. In some embodiments, the polypeptide is a fusion protein comprising the isolated HSV polypeptide fused to a heterologous polypeptide. Such fusion proteins can optionally be soluble fusion proteins. As indicated in FIG. 5C, some antigens of the invention elicit primarily CD4+ T cell reactions in HSV-infected subjects, while others elicit primarily CD8+ T cells reactions in HSV-infected subjects. Some HSV-1 antigens elicit both CD4 and CD8 T-cells in many subjects, and these antigens eliciting coordinated immune responses are considered especially valuable. Thus, in one embodiment, the HSV polypeptide is one that elicits both CD4 and CD8 responses. In one embodiment, the HSV polypeptide comprises multiple epitopes, as set forth in Table 4, wherein the epitopes may be from the same HSV protein or from more than one HSV protein. The HSV polypeptide comprising one or more epitopes of the invention can comprise a fragment of a full-length HSV protein, or the full-length HSV protein. In some embodiments, multiple HSV polypeptides are provided together within a single composition, within a kit, or within a larger polypeptide. In one embodiment, the invention provides a multi-epitopic or multi-valent vaccine.

The embodiments comprising multiple HSV polypeptides include any combination of two or more of the epitopes listed in Table 4 or the corresponding full-length proteins, and, optionally, additional HSV polypeptides of HSV-1 and/or HSV-2, including those described in United States patent publication number US-2010-0203073-A1, published on Aug. 12, 2010, namely, VP16, gK or gL, or fragments thereof that include amino acids 64-160, 90-99, 141-240, 187-199, 191-203, 215-227, 218-320, 219-230, 381-490, 479-489, 479-488, 480-488 or 477-490 of VP16 (UL48); 201-209 of glycoprotein K (UL53); or 66-74 of glycoprotein L (UL1).

In one embodiment, the HSV polypeptide comprises UL1, UL13, UL21, UL25, UL26, UL27, UL29, UL31, UL37, UL39, UL40, UL41, UL46, UL47, UL48, UL49, UL53, UL54, US1, US7, ICP0, ICP4, or any combination of two or more of the preceding polypeptides. The polypeptide can include the full-length of one or more of the HSV proteins, or a portion that includes one or more epitopes as described herein. In another embodiment, the HSV polypeptide comprises one or more epitopes selected from the group consisting of: amino acids 66-74 of UL1 (LIDGIFLRY; SEQ ID NO: 1), amino acids 512-520 of UL39 (YMESVFQMY; SEQ ID NO: 2), amino acids 259-268 of UL41 (HTDLHPNNTY; SEQ ID NO: 3), amino acids 354-362 of UL46 (ATDSLNNEY; SEQ ID NO: 4), amino acids 360-368 of UL47 (RSSLGSLLY; SEQ ID NO: 5), amino acids 566-574 of UL47 (FTAPEVGTY; SEQ ID NO: 6), amino acids 90-99 of UL48 (SALPTNADLY; SEQ ID NO: 7), amino acids 479-488 of UL48 (FTDALGIDEY; SEQ ID NO: 8), amino acids 201-209 of UL53 (ETDPVTFLY; SEQ ID NO: 9), amino acids 389-397 of UL13 (TLLELVVSV; SEQ ID NO: 10), amino acids 367-375 of UL25 (FLWEDQTLL; SEQ ID NO: 11), amino acids 280-288 of UL27 (SVYPYDEFV; SEQ ID NO: 12), amino acids 448-456 of UL27 (FLIAYQPLL; SEQ ID NO: 13), amino acids 425-433 of UL39 (RILGVLVHL; SEQ ID NO: 14), amino acids 184-192 of UL40 (ILIEGIFFA; SEQ ID NO: 15), amino acids 286-294 of UL47 (FLADAVVRL; SEQ ID NO: 16), amino acids 374-382 of UL47 (ALLDRDCRV; SEQ ID NO: 17), amino acids 545-553 of UL47 (RLLGFADTV; SEQ ID NO: 18), amino acids 162-170 of UL21 (VYTPSPYVF; SEQ ID NO: 19), amino acids 292-300 of UL31 (EYQRLYATF; SEQ ID NO: 20), amino acids 221-230 of UL37 (AYSLLFPAPF; SEQ ID NO: 21), amino acids 640-648 of UL37 (AYLPRPVEF; SEQ ID NO: 22), amino acids 226-234 of UL46 (AYVSVLYRW; SEQ ID NO: 23), amino acids 504-512 of UL54 (KYFYCNSLF; SEQ ID NO: 24), amino acids 1097-1106 of ICP4 (LYPDAPPLRL; SEQ ID NO: 25), amino acids 170-179 of UL25 (SSGVVFGTWY; SEQ ID NO: 26), amino acids 235-243 of UL25 (AVLCLYLLY; SEQ ID NO: 27), amino acids 22-30 of UL26 (YVAGFLALY; SEQ ID NO: 28), amino acids 326-334 of UL26 (YLWIPASHY; SEQ ID NO: 29), amino acids 295-303 of UL27 (VYMSPFYGY; SEQ ID NO: 30), amino acids 641-649 of UL27 (FTFGGGYVY; SEQ ID NO: 31), amino acids 460-468 of UL29 (ALLAKMLFY; SEQ ID NO: 32), amino acids 895-903 of UL29 (YMANQILRY; SEQ ID NO: 33), amino acids 93-101 of UL46 (LASDPHYEY; SEQ ID NO: 34), amino acids 126-134 of UL46 (AILTQYWKY; SEQ ID NO: 35), amino acids 224-232 of UL46 (LLAYVSVLY; SEQ ID NO: 36), amino acids 333-341 of UL46 SIVHHHAQY (SEQ ID NO: 37), amino acids 508-516 of UL47 ALATVTLKY (SEQ ID NO: 38), amino acids 698-706 of ICP0 VPGWSRRTL (SEQ ID NO: 39), amino acids 382-390 of UL21 VPRPDDPVL (SEQ ID NO: 40), amino acids 281-290 of UL49 RPTERPRAPA (SEQ ID NO: 41), amino acids 70-78 of US1 APRIGGRRA (SEQ ID NO: 42), amino acids 22-30 of US7 VVRGPTVSL (SEQ ID NO: 43), amino acids 97-105 of US7 CPRRPAVAF (SEQ ID NO: 44), and amino acids 195-203 of US7 APASVYQPA (SEQ ID NO: 45).

In another embodiment, the HSV polypeptide comprises one or more epitopes that have not been previously described as CD8 epitopes with the same proven or probable HLA restriction using PBMC from HSV-2-infected persons and HSV-2 peptides. For example, the HSV polypeptide comprises one or more epitopes selected from the group consisting of: amino acids 66-74 of UL1 (LIDGIFLRY; SEQ ID NO: 1), amino acids 259-268 of UL41 (HTDLHPNNTY; SEQ ID NO: 3), amino acids 360-368 of UL47 (RSSLGSLLY; SEQ ID NO: 5), amino acids 566-574 of UL47 (FTAPEVGTY; SEQ ID NO: 6), amino acids 90-99 of UL48 (SALPTNADLY; SEQ ID NO: 7), amino acids 479-488 of UL48 (FTDALGIDEY; SEQ ID NO: 8), amino acids 201-209 of UL53 (ETDPVTFLY; SEQ ID NO: 9), amino acids 389-397 of UL13 (TLLELVVSV; SEQ ID NO: 10), amino acids 280-288 of UL27 (SVYPYDEFV; SEQ ID NO: 12), amino acids 425-433 of UL39 (RILGVLVHL; SEQ ID NO: 14), amino acids 184-192 of UL40 (ILIEGIFFA; SEQ ID NO: 15), amino acids 286-294 of UL47 (FLADAVVRL; SEQ ID NO: 16), amino acids 374-382 of UL47 (ALLDRDCRV; SEQ ID NO: 17), amino acids 545-553 of UL47 (RLLGFADTV; SEQ ID NO: 18), amino acids 162-170 of UL21 (VYTPSPYVF; SEQ ID NO: 19), amino acids 292-300 of UL31 (EYQRLYATF; SEQ ID NO: 20), amino acids 221-230 of UL37 (AYSLLFPAPF; SEQ ID NO: 21), amino acids 640-648 of UL37 (AYLPRPVEF; SEQ ID NO: 22), amino acids 226-234 of UL46 (AYVSVLYRW; SEQ ID NO: 23), amino acids 504-512 of UL54 (KYFYCNSLF; SEQ ID NO: 24), amino acids 1097-1106 of ICP4 (LYPDAPPLRL; SEQ ID NO: 25), amino acids 170-179 of UL25 (SSGVVFGTWY; SEQ ID NO: 26), amino acids 235-243 of UL25 (AVLCLYLLY; SEQ ID NO: 27), amino acids 22-30 of UL26 (YVAGFLALY; SEQ ID NO: 28), amino acids 326-334 of UL26 (YLWIPASHY; SEQ ID NO: 29), amino acids 295-303 of UL27 (VYMSPFYGY; SEQ ID NO: 30), amino acids 641-649 of UL27 (FTFGGGYVY; SEQ ID NO: 31), amino acids 460-468 of UL29 (ALLAKMLFY; SEQ ID NO: 32), amino acids 895-903 of UL29 (YMANQILRY; SEQ ID NO: 33), amino acids 93-101 of UL46 (LASDPHYEY; SEQ ID NO: 34), amino acids 126-134 of UL46 (AILTQYWKY; SEQ ID NO: 35), amino acids 224-232 of UL46 (LLAYVSVLY; SEQ ID NO: 36), amino acids 333-341 of UL46 SIVHHHAQY (SEQ ID NO: 37), amino acids 508-516 of UL47 ALATVTLKY (SEQ ID NO: 38), amino acids 382-390 of UL21 VPRPDDPVL (SEQ ID NO: 40), amino acids 281-290 of UL49 RPTERPRAPA (SEQ ID NO: 41), amino acids 70-78 of US1 APRIGGRRA (SEQ ID NO: 42), amino acids 22-30 of US7 VVRGPTVSL (SEQ ID NO: 43), amino acids 97-105 of US7 CPRRPAVAF (SEQ ID NO: 44), and amino acids 195-203 of US7 APASVYQPA (SEQ ID NO: 45).

In another embodiment, the HSV polypeptide comprises one or more type-specific HSV-1 (versus HSV-2) epitopes as identified in Table 4. In an alternative embodiment, the HSV polypeptide comprises one or more type-common (HSV-1 and HSV-2) epitopes as identified in Table 4. In a further embodiment, the HSV polypeptide comprises a combination of type-common and type-specific epitopes. In another embodiment, the HSV polypeptide comprises one or more of the epitopes identified as recognized by T cells of the human trigeminal ganglia, including epitopes of VP16 (gene UL48), immediate early proteins UL39 and ICP0, and late glycoproteins K and L, alone or in combination with one or more of the polypeptides disclosed herein. In one embodiment, the HSV polypeptide comprises epitopes of VP16/UL48, UL39 and/or ICP0.

In some embodiments, the selection of a combination of epitopes and/or antigens to be included within a single composition and/or polypeptide is guided by optimization of population coverage with respect to HLA alleles. For example, each epitope restricted by HLA allele A*0201 will cover 40-50% of most ethnic groups. By adding epitopes restricted by A*0101 (20%), A*2402 (˜5-25%), B*0702 (10-15%), and A*29 (5-10%), one can, in the aggregate, cover more people. In one embodiment, the HSV polypeptide comprises one or more of the epitopes identified in Table 4 as associated with HLA allele A*0101. In another embodiment, the HSV polypeptide comprises one or more of the epitopes identified in Table 4 as associated with HLA allele A*0201. In another embodiment, the HSV polypeptide comprises one or more of the epitopes identified in Table 4 as associated with HLA allele A*2402. In another embodiment, the HSV polypeptide comprises one or more of the epitopes identified in Table 4 as associated with HLA allele A*2902. In another embodiment, the HSV polypeptide comprises one or more of the epitopes identified in Table 4 as associated with HLA allele B*0702. In a further embodiment, the HSV polypeptide comprises epitopes identified in Table 4 as associated with 2, 3, 4 or all 5 of the HLA alleles, A*0101, A*0201, A*2402, A*2902, and B*0702. As is understood by those skilled in the art, these HLA alleles, or HLA alleles that are biologically expected to bind to peptide epitopes restricted by these HLA alleles, cover 80-90% of the human population in most major ethnic and racial groups.

In one embodiment, the HSV polypeptide comprises all of UL1, UL13, UL21, UL25, UL26, UL27, UL29, UL31, UL37, UL39, UL40, UL41, UL46, UL47, UL48, UL49, UL53, UL54, US1, US7, ICP0, and ICP4, not necessarily in that order. In another embodiment, the HSV polypeptide comprises all of the epitopes listed in Table 4, not necessarily in the order listed.

In one embodiment, the invention provides UL39 and UL48, optionally in combination with UL46 and/or UL40, as full-length proteins and/or as fragments thereof that include one or more epitopes identified in Table 4. In one embodiment, the invention provides UL25, UL39 and UL47, as full-length proteins and/or as fragments thereof that include one or more epitopes identified in Table 4. In one embodiment, the invention provides UL25 and UL47, as full-length proteins and/or as fragments thereof that include one or more epitopes identified in Table 4. In one embodiment, the invention provides UL25 and UL39, as full-length proteins and/or as fragments thereof that include one or more epitopes identified in Table 4. In one embodiment, the invention provides UL39 and UL47, as full-length proteins and/or as fragments thereof that include one or more epitopes identified in Table 4. In one embodiment, the invention provides UL46, UL47, UL49, and/or UL21, as full-length proteins and/or as fragments thereof that include one or more epitopes identified in Table 4. In one embodiment, the invention provides UL39 and/or UL46, as full-length proteins and/or as fragments thereof that include one or more epitopes identified in Table 4. The selection of particular combinations of antigens and/or epitopes can be guided by the data described in Example 1, including that presented in FIGS. 4 and 5. For example, antigens that exhibit desirable characteristics per FIG. 4 and/or those that include multiple immunogenic epitopes can be combined in a single composition and/or polypeptide.

In each of the embodiments described herein, the HSV polypeptide, or epitope thereof, may be present alone or in combination with other epitopes listed in Table 4, or with other epitopes of HSV-1 or HSV-2; as a single contiguous polypeptide, or as a composition or kit comprising multiple polypeptides. For embodiments in which the epitopes are present as a single continuous polypeptide, those skilled in the art will appreciate that the epitopes may be adjacent to one another, or present as epitopes separated by short linker sequences selected to enhance epitope release during antigen processing in cells. For example, in one embodiment, the polypeptide consists of one or more of the HSV-1 proteins selected from the group consisting of UL1, UL13, UL21, UL25, UL26, UL27, UL29, UL31, UL37, UL39, UL40, UL41, UL46, UL47, UL48, UL49, UL53, UL54, US1, US7, ICP0, and ICP4, optionally, up to 100 amino acid residues of linker sequence between said proteins. In another example, the polypeptide consists of one or more of the epitopes listed in Table 4 and, optionally, up to 100 amino acid residues of linker sequence between said eptiopes. Typically, a linker comprises up to 10, up to 50, or up to 100 amino acid residues. One skilled in the art can appreciate the appropriate options for selecting a linker sequence.

In one embodiment, the invention provides a vector comprising a polynucleotide encoding an HSV polypeptide of the invention. Also provided is a host cell transformed with the vector, as well as a method of producing a HSV-1 polypeptide comprising culturing the host cell and recovering the polypeptide so produced. The invention additionally provides a HSV polypeptide produced by the aforementioned method. Also provided is a recombinant virus genetically modified to express a HSV polypeptide of the invention, including, for example, an adenovirus or poxvirus.

The diseases to be prevented or treated using compositions and methods of the invention include diseases associated with herpes virus infection, particularly HSV-1 infection. HSV-1 infections have considerable medical impact. Highlights include neonatal HSV-1 encephalitis and visceral infection leading to death or brain damage, HSV-1 encephalitis in adults, and a wide spectrum of HSV eye infections including acute retinal necrosis (ARN) and herpetic stromal keratitis (HSK). In addition, some compositions of the invention are suitable for treating or preventing conditions resulting from infection with HSV-1 and conditions resulting from infection with HSV-2. Such compositions can be administered to patients who may be or may become infected with either or both HSV-1 and HSV-2.

The invention additionally provides pharmaceutical compositions comprising the HSV antigens and epitopes identified herein. Also provided is an isolated polynucleotide that encodes a polypeptide of the invention, and a composition comprising the polynucleotide. The invention additionally provides a recombinant virus genetically modified to express a polynucleotide of the invention, and a composition comprising the recombinant virus. In one embodiment, the recombinant virus is vaccinia virus, canary pox virus, HSV, lentivirus, retrovirus or adenovirus. A composition of the invention can be a pharmaceutical composition. The composition can optionally comprise a pharmaceutically acceptable carrier and/or an adjuvant.

### BRIEF DESCRIPTION OF THE FIGURES

FIGS. 1A-1B are scatterplots that illustrate use of CD137 to detect and enrich HSV-1-specific CD8s from blood. FIG. 1A: Gated live, CD3+, lymphocyte forward/side scatter window cells analyzed for CD8 and CD137 after admixing selected CD8+ cells with autologous moDC loaded with mock or HSV-1-infected HeLa cell debris. Numbers are percentages of cells in indicated quadrants. Data for HSV-1 infected participant 1, and HSV-uninfected participant 12 are shown. Small boxes indicate approximate gates for FACS. FIG. 1B: Reactivity of participant 1 polyclonal expanded responder cell line derived from CD3+ CD8+CD137^{high} or CD137^{low} cells after exposure to mock- or HSV-1 infected autologous B-LCL or control stimuli for 18 h. Responder cells were initially gated using dump-gating of CFSE-labeled APC and for CD3 and CD8 (left), and analyzed for intracellular IFN-γ. Numbers are percentages of cells in indicated quadrants.

FIGS. 2A-2B plot representative data from participant 1 (Table 1) for CD8 T-cell reactivity with HSV-1 ORFs and peptides. FIG. 2A shows IFN-γ secretion by polyclonal expanded responder cell line derived from CD3+ CD137^{high} cells exposed to artificial APC expressing the indicated HLA molecules and the HSV-1 ORFs arrayed in nominal genomic order on the X-axis (ORFs, from left to right along X-axis, full-length unless otherwise indicated: RL1 (g34.5), RL2 (ICP0) fragment A, RL2 (ICP0) fragment B, RL2 (ICP0) fragment C, UL1, UL2, UL3, UL4, UL5, UL6, UL7, UL8, UL9 fragment A, UL9 fragment B, UL9 fragment C, UL10, UL11, UL12, UL13, UL14, UL15, UL16, UL17, UL18, UL19, UL20, UL20 fragment A, UL21, UL22, UL23, UL24, UL25, UL26, UL26 fragment A, UL26 fragment B, UL26.5, UL27, UL28 fragment A, UL28 fragment B, UL29, UL30, UL31, UL32, UL33, UL34, UL34 fragment A, UL35, UL36 fragment A, UL36 fragment B, UL36 fragment C, UL36 fragment D, UL37 fragment A, UL37 fragment B, UL38, UL39, UL40, UL40 fragment A, UL40 fragment B, UL41, UL42, UL43, UL43 fragment A, UL43 fragment B, UL44, UL45, UL46, UL47, UL48, UL49.5, UL49, UL50, UL51, UL52, UL53, UL53 fragment A, UL54, UL55, UL56, US1, US2, US3, US4, US4 fragment A, US5, US6, US7, US7 fragment B, US8, US8 fragment A, US8 fragment B, US8.5, UL9, US10, US11, US12, RS1 (ICP4) full, RS1 (ICP4) fragment A, RS1 (ICP4) fragment B, RS1 (ICP4) fragment D; final 3 bars are media. The names of HSV-1 ORFs driving two representative positive responses are labeled for HLA A*0101 in the first panel. FIG. 2B dot-plots are representative analyses of polyclonal HSV-1-reactive CD8 cells from the same person probed for reactivity with individual HSV-1 peptides derived from these ORFs, indicated at bottom, or negative or positive controls shown at left. Numbers are percentages of cells in the upper right quadrants.

FIG. 3 (2 panels) depicts HLA allele- and HSV-1 ORF-level IFN-γ immune signature of CD8 cells in PBMC of seven humans infected with HSV-1. Participant identities are listed at bottom. Each column represents an individual HLA-ORFeome screen. The HLA A, B, or C alleles used are indicated at the top. Each row represents the integrated data for an HSV-1 ORF, with ORFs listed in nominal genomic order from RL 1 (encoding γ34.5 protein) at top to RS1 (encoding protein ICP4) at bottom with gene names from Genbank NC_{—}001806.1. A red cell shows that specific IFN-γ secretion was detected for the intersecting HLA allele and HSV-1 ORF. Data for ORFs expressed as more than one fragment or exon are simplified to a yes/no call. UL36 amino acid coverage was partial (see text). Black arrows indicate, from top to bottom, genes UL39, UL46, and US6.

FIG. 4 is a graphical summary of direct PBMC IFN-γ ELISPOT. HSV-1 peptides from Table 4 (n=40, detailed in Example 1) were tested with PBMC from 20 persons with HSV-1 infection. Each column is one participant; 1-7 are detailed in Table 1. Each row lists a peptide with one or more positive ELISPOT results. Light-colored cells represent possible reactivities based on the participant bearing an HLA allele corresponding to the peptide tested, but for which the ORFeome screen with the listed HLA allele and ORF (see Table 4 for HLA allele), and peptide ELISPOT, were negative. Darkest cells are similar, but the ORFeome screen was positive and the peptide ELISPOT with the indicated peptide was negative. Medium-dark cells represent ORFs for which both the ORFeome screen with the listed HLA allele and ORF, and the ELISPOT for at least one peptide in the ORF, were positive. Numbers in these cells are positive ELISPOT results in units of net IFN-γ SFU/10⁶ PBMC.

FIGS. 5A-5C illustrate CD4 and integrated T-cell reactivity to the HSV-1 ORFeome by PBMC from HSV-1-infected humans. FIG. 5A: Left two panels show expression of CD137 by PBMC after 20 h of exposure to whole cell-associated UV-killed mock or HSV-1 lysate. Dot-plots were gated for live, CD3+ cells in the lymphocyte forward/side scatter region. Numbers are the percent of cells in the upper right quadrant. Small boxes indicate approximate gates for FACS. Right 6 panels show reactivity of polyclonal twice-expanded responder cell lines derived from CD3+ CD4+ CD137^{high} or CD3+ CD4+ CD137^{low} cells, after 18 h re-exposure to autologous APC and whole cell-associated UV-killed mock or HSV-1 lysate. APC were CFSE dump-gated and responder cells stained intracellularly for IFN-γ and IL-2. Numbers are percentages of cells in indicated quadrants. FIG. 5B: Representative screen of polyclonal HSV-1-reactive CD4 cells to each HSV-1 protein or fragment, whole HSV-1 positive control, and irrelevant microbial negative control proteins. Data are mean of duplicate assays. Horizontal line is cutoff for positive responses calculated as described in Methods. ORFs, from left to right along X-axis, full-length unless otherwise indicated: RL1 (g34.5), RL2 (ICP0) fragment A, RL2 (ICP0) fragment B, RL2 (ICP0) fragment C, UL1, UL2, UL3, UL4, UL5, UL6, UL7, UL8, UL9 fragment A, UL9 fragment B, UL9 fragment C, UL10, UL11, UL12, UL13, UL14, UL15, UL16, UL17, UL18, UL19, UL20, UL21, UL22, UL23, UL24, UL25, UL26, UL26.5, UL27, UL28 fragment A, UL28 fragment B, UL29, UL30, UL31, UL32, UL33, UL34, UL35, UL36 fragment A, UL36 fragment B, UL36 fragment D, UL37 fragment A, UL37 fragment B, UL38, UL39, UL40, UL41, UL42, UL43, UL44, UL45, UL46, UL47, UL48, UL49, UL49.5, UL50, UL51, UL52, UL53, UL54, UL55, UL56, US1, US2, US3, US4, US5, US6, US7, USB, US8.5, UL9, US10, US11, US12, RS1 (ICP4), RS1 (ICP4) fragment A, RS1 (ICP4) fragment B, RS1 (ICP4) fragment D; next two bars are mock and UV HSV1, respectively, followed by negative controls: p203 (5 bars), no DNA (6 bars), microbial proteins (15 bars). FIG. 5C (2 panels): Graphical representation of CD4 and CD8 reactivity to HSV-1 ORFs in PBMC from seven HSV-1-infected humans, indicated in the rows. Each column is an HSV-1 ORF. The ORFs are grouped by their kinetic expression during viral replication. Color code is CD8 only (dark squares), CD4 only (medium squares), CD8 and CD4 (light squares); white (blank) indicates no reactivity.

FIG. 6 is a schematic overview of the high throughput T-cell antigen discovery pathway described in Example 1. In this example, the microbe (left) was a virus, HSV-1, but the workflow can also be adapted to bacteria or parasites. A microbial ORF library is initially cloned in a flexible format and subcloned into both a custom protein expression vector for CD4 assays, and into a custom transient expression vector for CD8 assays. The CD4 workflow (upper section) stimulates PBMCs with whole killed microbe, and detects and isolates microbe-specific CD4 T-cells based on differential CD137 expression followed by expansion. The polyclonal CD4 responder cells are then assayed against the protein library made in vitro from the secondary protein expression clone set. The CD8 workflow (lower section) uses cross-presentation by microbial antigen-laden DC to stimulate CD8 T-cells from PBMCs. After CD137-based selection and expansion, effector cells are assayed for reactivity with panels of person-specific artificial APCs made by co-transfection of Cos7 cells with participant HLA class I cDNA and the transient transfection microbial ORF set. The integrated assays assign each ORF a yes/no result for each participant for CD4 and CD8 T-cells.

FIGS. 7A-7E demonstrate reactivity of polyclonal expanded CD8 cell lines derived from CD3+ CD137^{high} cells with synthetic HSV-1 peptides at 1 μg/ml. These data are the basis for Table 4. Autologous PBMC used as APC were CFSE-labeled and dump-gated. Each one of FIGS. 7A-7E shows results from a distinct participant from Table 1, and begins with negative control DMSO stimulation and positive control SEB stimulation (controls are the top dot-plots in each panel), followed by peptides. Each dot-plot shows expression of CD8 and intracellular IFN-γ. The identity of the peptides is indicated below each dot-plot, with asterisks after three HLA A*0101-restricted peptides studied in both participants 1 and 2. The identity of the HLA allele used to assign restriction is indicated above the relevant dot-plots. The numbers are the percentages of cells in the upper right quadrants of each dot-plot.

### DETAILED DESCRIPTION OF THE INVENTION

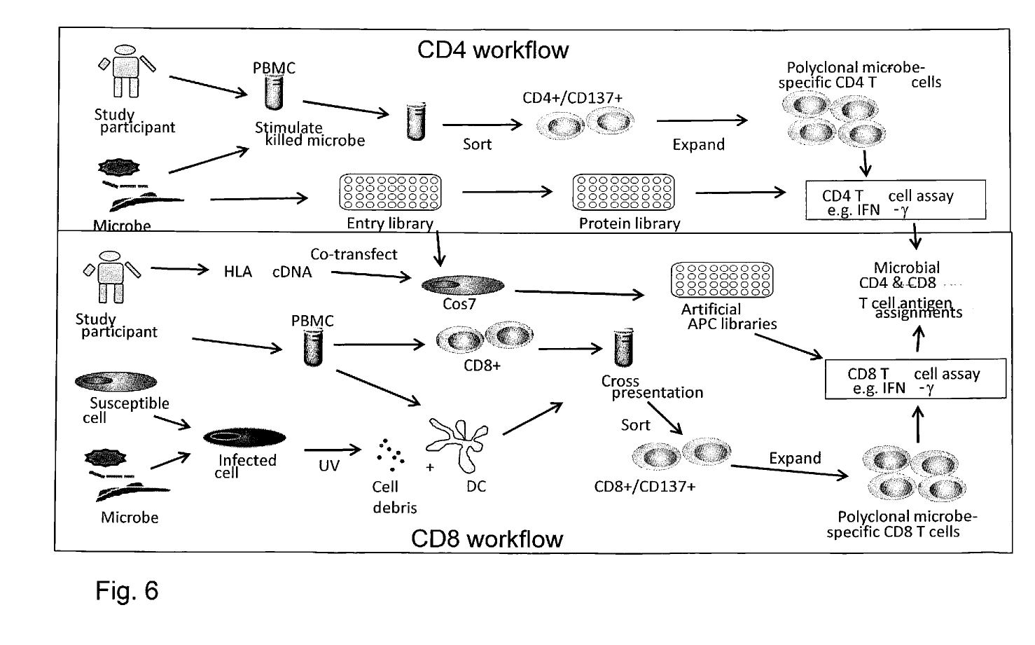

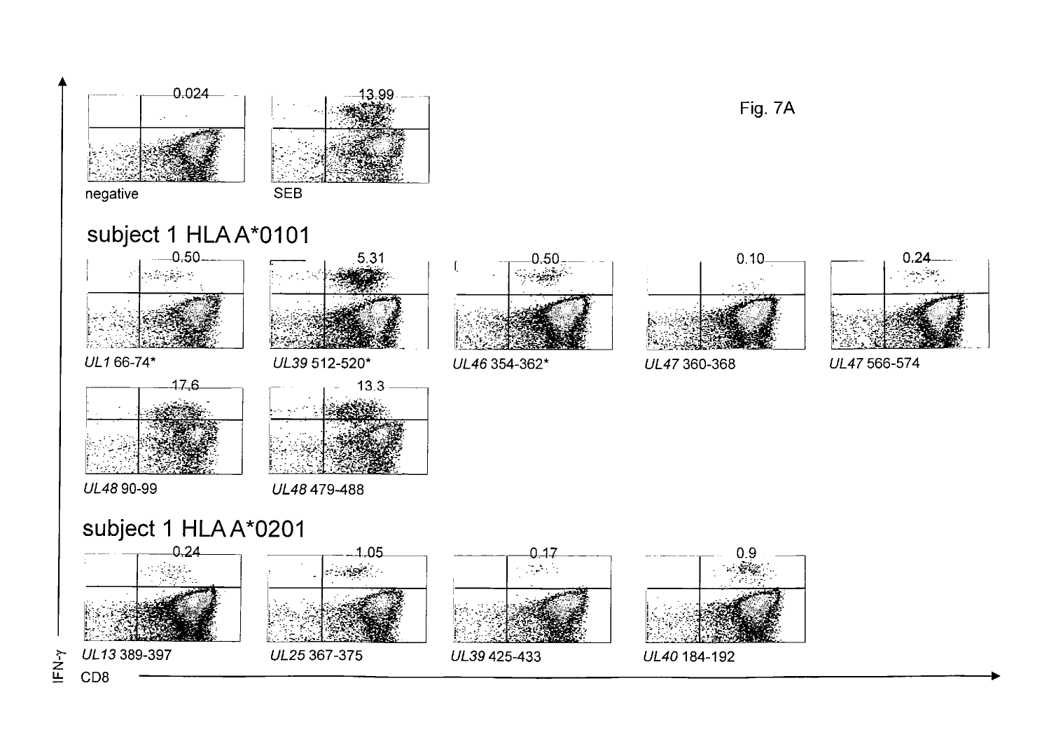

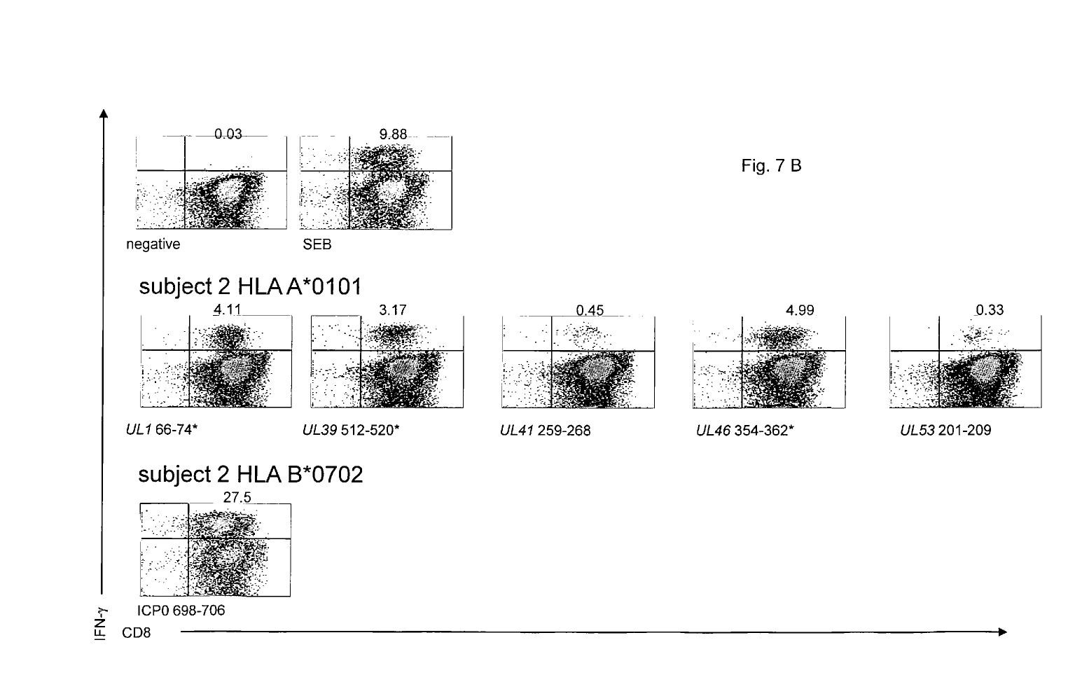

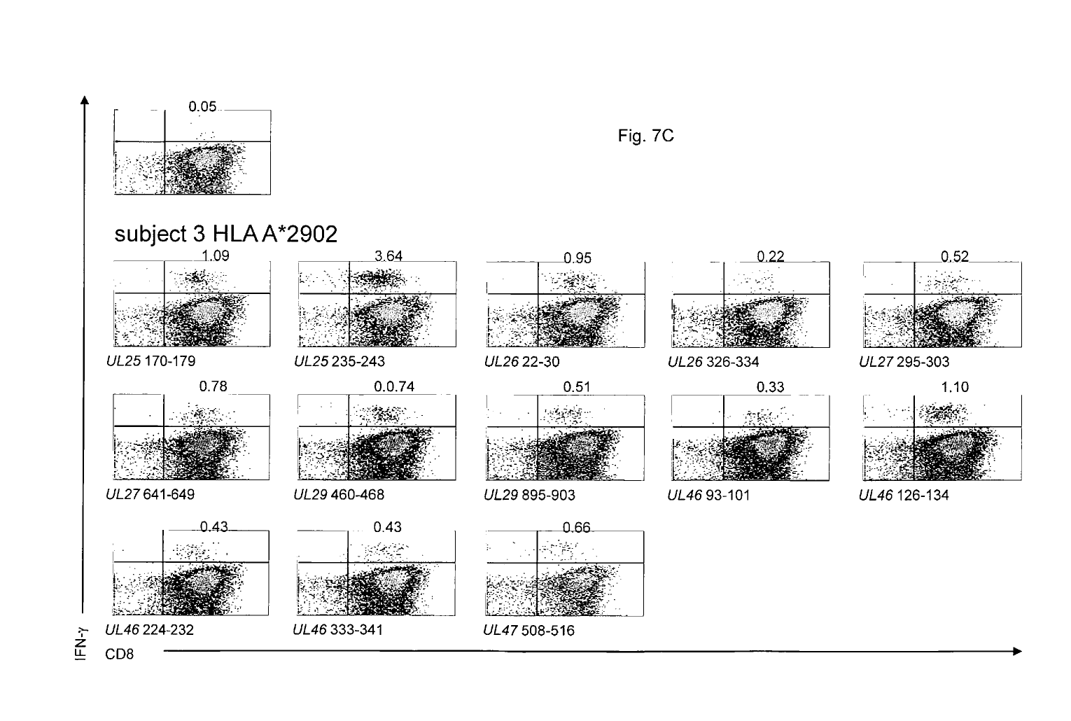

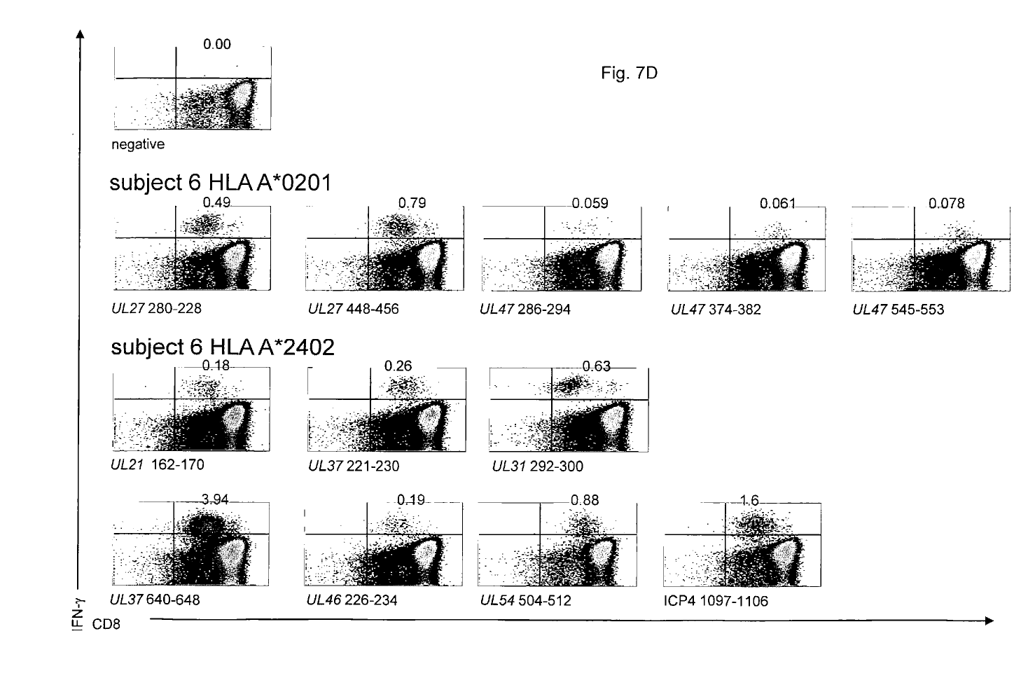

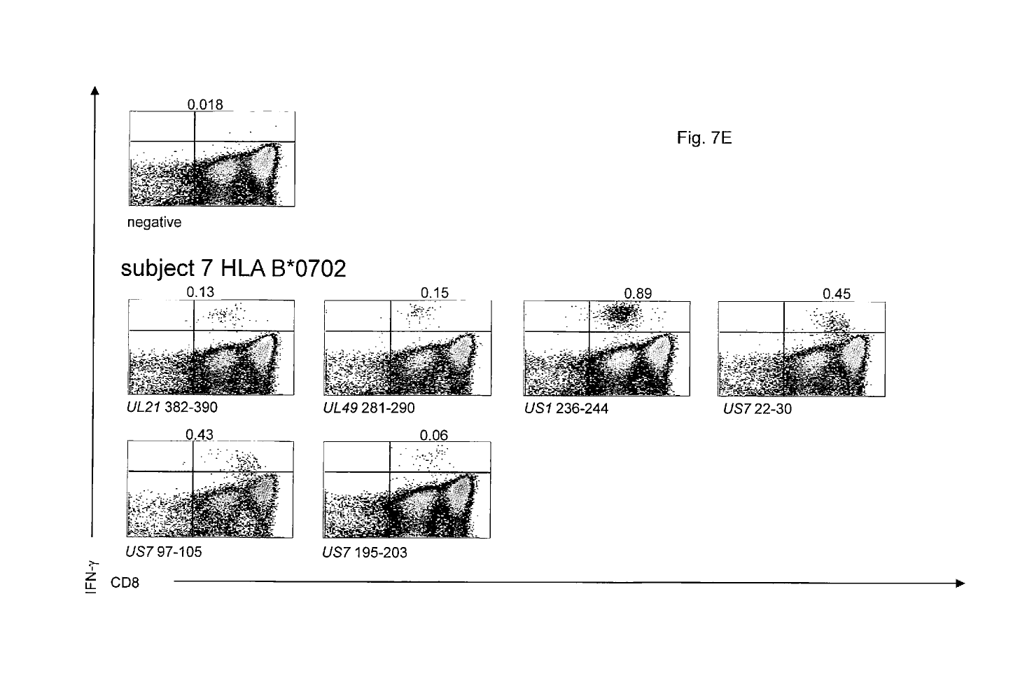

The invention described herein is based on the discovery of the HSV-1 open reading frames (antigens) and minimal units of recognition (epitopes) recognized by CD8 and CD4 T-cells in the TG of humans as revealed by cross-presentation and genome-wide screening. An established expression cloning technology (Koelle et al. J. Immunol. 2001; Jing et al. J Immunol. 2005) was considerably adapted and improved to determine which HSV-1 open reading frames were recognized. In the new workflow (FIG. 6), we initially used full length HSV-1 genes, not fragments as in the earlier publications In addition, we used a novel method based on cross-presentation of HSV-1-infected cell debris, and on selection of rare HSV-1-specific CD8 T cells from blood using selective expression of CD137, to enrich these very rare cells from blood samples. The novel cell enrichment step gave the genome-wide screening a sufficient signal to noise ratio to determine which genes or large fragments of genes were positive.

Immune system cells that can monitor, surveil and control HSV-1 reactivation at its site of origin, infected neurons in the TG, offer effective targets for vaccines. In a preventative mode, pre-equipping a patient with T-cells specific for those HSV-1 proteins that are expressed in TG could modify (reduce) initial and recurrent infection of TG neurons. In a therapeutic mode, a vaccine would boost levels of T-cells that are capable of sensing HSV-1 reactivation in TG neurons, and thereby down-regulate recurrent infection.

### DEFINITIONS

All scientific and technical terms used in this application have meanings commonly used in the art unless otherwise specified. As used in this application, the following words or phrases have the meanings specified.

As used herein, “polypeptide” includes proteins, fragments of proteins, and peptides, whether isolated from natural sources, produced by recombinant techniques or chemically synthesized. Polypeptides of the invention typically comprise at least about 6 amino acids, and can be at least about 15 amino acids. Typically, optimal immunological potency is obtained with lengths of 8-10 amino acids. Those skilled in the art also recognize that additional adjacent sequence from the original (native) protein can be included, and is often desired, in an immunologically effective polypeptide suitable for use as a vaccine. This adjacent sequence can be from 1, 2, 3, 4, 5, 6, 7, 8, 9 or 10 amino acids in length to as much as 15, 20, 25, 30, 35, 40, 45, 50, 75 or 100 amino acids in length or more. Adjacent native sequence may be included at one, both or neither end of the identified epitope for use in a vaccine composition.

As used herein, particularly in the context of polypeptides of the invention, “consisting essentially of” means the polypeptide consists of the recited amino acid sequence and, optionally, adjacent amino acid sequence, but less than the full-length protein from which the polypeptide is derived. The adjacent sequence typically consists of additional, adjacent amino acid sequence found in the full length antigen, but variations from the native antigen can be tolerated in this adjacent sequence while still providing an immunologically active polypeptide.

As used herein, “epitope” refers to a molecular region of an antigen capable of eliciting an immune response and of being specifically recognized by the specific immune T-cell produced by such a response. Another term for “epitope” is “determinant” or “antigenic determinant”. Those skilled in the art often use the terms epitope and antigen interchangeably in the context of referring to the determinant against which an immune response is directed. A minimal epitope is the shortest antigenic region identified for a given antigenic polypeptide.

As used herein, “HSV polypeptide” includes HSV-1 and HSV-2, unless otherwise indicated. References to amino acids of HSV-1 proteins or polypeptides are based on the genomic sequence information regarding HSV-1 (strain 17+) as described in McGeoch et al., 1988, J. Gen. Virol. 69:1531-1574 (Genbank NC_{—}001806.1). References to amino acids of HSV-2 proteins or polypeptides are based on the genomic sequence information regarding HSV-2 as described in A. Dolan et al., 1998, J. Virol. 72(3):2010-2021 (Genbank NC_{—}001798.1).

As used herein, “substitutional variant” refers to a molecule having one or more amino acid substitutions or deletions in the indicated amino acid sequence, yet retaining the ability to be “immunologically active”, or specifically recognized by an immune cell. The amino acid sequence of a substitutional variant is preferably at least 80% identical to the native amino acid sequence, or more preferably, at least 90% identical to the native amino acid sequence. Typically, the substitution is a conservative substitution.

One method for determining whether a molecule is “immunologically active”, “immunologically effective”, or can be specifically recognized by an immune cell, is the cytotoxicity assay described in D. M. Koelle et al., 1997, Human Immunol. 53:195-205. Other methods for determining whether a molecule can be specifically recognized by an immune cell are described in the examples provided herein below, including the ability to stimulate secretion of interferon-gamma or the ability to lyse cells presenting the molecule. An immune cell will specifically recognize a molecule when, for example, stimulation with the molecule results in secretion of greater interferon-gamma than stimulation with control molecules. For example, the molecule may stimulate greater than 5 pg/ml, or preferably greater than 10 pg/ml, interferon-gamma secretion, whereas a control molecule will stimulate less than 5 pg/ml interferon-gamma.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “antigen-presenting cell” or “APC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells are a preferred type of antigen presenting cell. Dendritic cells are found in many non-lymphoid tissues but can migrate via the afferent lymph or the blood stream to the T-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods. For example, the APCs can be modified by exposure to the isolated antigen, alone or as part of a mixture, peptide loading, or by genetically modifying the APC to express a polypeptide that includes one or more epitopes.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, nickel, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “adjuvant” includes those adjuvants commonly used in the art to facilitate the stimulation of an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mich.); Merck Adjuvant 65 (Merck and Company, Inc., Rahway, N.J.); AS-2 (Smith-Kline Beecham); QS-21 (Aquilla); MPL or 3d-MPL (Corixa Corporation, Hamilton, Mont.); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, −7 or −12) and immunostimulatory DNA sequences. In some embodiments, such as with the use of a polynucleotide vaccine, an adjuvant such as a helper peptide or cytokine can be provided via a polynucleotide encoding the adjuvant.

As used herein, “a” or “an” means at least one, unless clearly indicated otherwise.

As used herein, to “prevent” or “protect against” a condition or disease means to hinder, reduce or delay the onset or progression of the condition or disease.

### OVERVIEW

Herpes simplex virus type 1 (HSV-1) and HSV-2 are related alphaherpesviruses. Each has about 85 known ORFs. HSV-1/HSV-2 amino acid identity ranges from 20 to 90% depending on the ORF. Animal gene knockout and monoclonal antibody (mAb) blocking studies, and data from immune-suppressed humans, suggest vital roles for CD4 and CD8 T-cells in the control of primary and recurrent HSV. CD8 T-cells usually recognize unmodified 8-10 amino acid epitopes. T-cell clonotypes can be either type-common, recognizing HSV-1 and HSV-2, or type-specific. Most HSV-specific CD8 and CD4 T-cell epitopes to date have been type-specific. Human HSV T-cell research has concentrated on HSV-2. This invention concerns the less-studied human T-cell response to HSV-1.

HSV infections are thought to be permanent, due to infection of sensory ganglion neurons. Infection is most accurately diagnosed by IgG serology: patients remain seropositive for life. The prevalence of HSV-1 infection is about 60% in diverse human populations. There is a great spectrum in the severity of HSV infections. Only a minority of persons with HSV corneal infection progress to blinding HSK. This is likely attributable at least in part to bona fide biological variation. Inoculum size is important in some HSV animal models. Inter-strain sequence divergence is of uncertain clinical significance. In general, there is so little sequence divergence between clinical strains that the large majority of epitope sequences described herein are expected to be identical in all or most circulating HSV-1 strains in the community. Divergent clinical severities in persons proven to have the same HSV strain argues a dominant effect. The invention addresses a need for treatment and prevention of HSV-1 infection.

### HSV Polypeptides

In one embodiment, the invention provides an isolated HSV polypeptide that comprises an epitope identified in Table 4. In some embodiments, the HSV polypeptide comprises additional adjacent native sequence from the corresponding full-length protein, up to and/or including the full-length sequence. The sequences of the HSV-1 antigens described herein and containing the epitopes listed in Table 4 can be found in Genbank NC_{—}001806, and are reproduced below.

| UL1/Glycoprotein L | |
|---|---|
| (SEQ ID NO: 46) | |
| 1 | mgilgwvgli avgilcvrgg lpsteyvirs rvarevgdil kvpcvplpsd dldwryetps |
| 61 | ainyalidgi flryhcpgld tvlwdrhaqr aywvnpflfg agfledlshp afpadtqete |
| 121 | trlalykeir qaldsrkqaa shtpvkagcv nfdysrtrrc vgrqdlgltn rtsgrtpvlp |
| 181 | sddeaglqpk plttpspiia tsdptprrda atksrrrrph frgl |
| UL13 | |
| (SEQ ID NO: 47) | |
| 1 | mdesrrqrpa ghvaanlspq garqrsfkdw lasyvhsnph gasgrpsgps lqdaaysrss |
| 61 | hgsrhrsglr erlraglsrw rmsrsshrra spetpgtaak lnrpplrrsq aaltappssp |
| 121 | shiltltrir klcspvfain palhyttlei pgarsfggsg gygdvqlire hklavktike |
| 181 | kewfavelia tllvgecvlr agrthnirgf iaplgfslqq rqlvfpaydm dlgkylgqla |
| 241 | slrttnpsvs talhqcftel aravvflntt cgishldikc anilvmlrsd ayslrravla |
| 301 | dfslvtlnsn stiargqfcl qepdlksprm fgmptaltta nfhtlvghgy nqppellvky |
| 361 | lnneraeftn hrlkhdvgla vdlyalgqtl lelvvsvyva pslgvpvtrf pgyqyfnnql |
| 421 | spdfalalla yrcvlhpalf vnsaetnthg laydvpegir rhlrnpkirr aftdrcinyq |
| 481 | hthkailssv alppelkpll vlvsrlchtn pcarhals |
| UL21 | |
| (SEQ ID NO: 48) | |
| 1 | melsyattmh yrdvvfyvtt drnrayfvcg gcvysvgrpc asqpgelakf glvvrgtgpd |
| 61 | drvvanyvrs elrqrglqdv rpigedevfl dsvcllnpnv sseldvintn dvevldecla |
| 121 | eyctslrtsp gvlisglrvr aqdriielfe hptivnvssh fvytpspyvf alaqahlprl |
| 181 | psslealvsg lfdgipaprq pldahnprtd vvitgrrapr piagsgagsg gagakratvs |
| 241 | efvqvkhidr vgpagvspap ppnntdsssl vpgaqdsapp gptlrelwwv fyaadralee |
| 301 | pradsgltre evravrgfre qawklfgsag aprafigaal glsplqklav yyyiihrerr |
| 361 | lspfpalvrl vgrytqrhgl yvprpddpvl adainglfrd alaagttaeq llmfdllppk |
| 421 | dvpvgsdvqa dstallrfie sqrlavpggv ispehvaylg aflsvlyagr grmsaathta |
| 481 | rltgvtslvl avgdvdrlsa fdrgaagaas rtraagyldv lltvrlarsq hgqsv |
| UL25 | |
| (SEQ ID NO: 49) | |
| 1 | mdpycpfdal dvwehrrfiv adsrnfitpe fprdfwmspv fnlpretaae qvvvlqaqrt |
| 61 | aaaaalenaa mqaaelpvdi errlrpiern vheiagalea letaaaaaee adaargdepa |
| 121 | gggdggappg lavaemevqi vrndpplryd tnlpvdllhm vyagrgatgs sgvvfgtwyr |
| 181 | tiqdrtitdf plttrsadfr dgrmsktfmt alvlslqacg rlyvgqrhys afecavlcly |
| 241 | llyrnthgaa ddsdrapvtf gdllgrlpry laclaavigt eggrpqyryr ddklpktqfa |
| 301 | agggryehga lashiviatl mhhgvlpaap gdvprdasth vnpdgvahhd dinraaaafl |
| 361 | srghnlflwe dqtllratan titalgviqr llangnvyad rlnnrlqlgm lipgavpsea |
| 421 | iargasgsds gaiksgdnnl ealcanyvlp lyradpavel tqlfpglaal cldaqagrpv |
| 481 | gstrrvvdms sgarqaalvr ltalelinrt rtnptpvgev ihandalaiq yeqglgllaq |
| 541 | qariglgsnt krfsafnvss dydmlyflcl gfipqylsav |
| UL26 | |
| (SEQ ID NO: 50) | |
| 1 | maadapgdrm eeplpdravp iyvagflaly dsgdsgelal dpdtvraalp pdnplpinvd |
| 61 | hragcevgrv lavvddprgp ffvgliacvq lervletaas aaiferrgpp lsreerllyl |
| 121 | itnylpsysl atkrlggeah pdrtlfahva lcaigrrlgt ivtydtglda aiapfrhlsp |
| 181 | asregarrla aeaelalsgr twapgvealt htllstavnn mmlrdrwslv aerrrqagia |
| 241 | ghtylqasek fkmwgaepvs apargyknga pestdippgs iaaapqgdrc pivrqrgval |
| 301 | spvlppmnpv ptsgtpapap pgdgsylwip ashynqlvag haapqpqphs afgfpaaags |
| 361 | vaygphgagl sqhypphvah qypgvlfsgp spleaqiaal vgaiaadrqa ggqpaagdpg |
| 421 | vrgsgkrrry eagpsesycd qdepdadypy ypgeargapr gvdsrraarh spgtnetita |
| 481 | lmgavtslqq elahmrarts apygmytpva hyrpqvgepe pttthpalcp peavyrppph |
| 541 | sapygppqgp ashaptppya paacppgppp ppcpstqtra plptepafpp aatgsqpeas |
| 601 | naeagalvna ssaahvdvdt araadlfvsq mmgar |
| UL27/Glycoprotein B | |
| (SEQ ID NO: 51) | |
| 1 | mrqgapargr rwfvvwallg ltlgvlvasa apsspgtpgv aaatqaangg patpappapg |
| 61 | apptgdpkpk knrkpkppkp prpagdnatv aaghatlreh lrdikaentd anfyvcpppt |
| 121 | gatvvqfeqp rrcptrpegq nytegiavvf keniapykfk atmyykdvtv sqvwfghrys |
| 181 | qfmgifedra pvpfeevidk inakgvcrst akyvrnnlet tafhrddhet dmelkpanaa |
| 241 | trtsrgwhtt dlkynpsrve afhrygttvn civeevdars vypydefvla tgdfvymspf |
| 301 | ygyregshte htsyaadrfk qvdgfyardl ttkaratapt trnllttpkf tvawdwvpkr |
| 361 | psvctmtkwq evdemlrsey ggsfrfssda isttfttnlt eyplsrvdlg dcigkdarda |
| 421 | mdrifarryn athikvgqpq yylanggfli ayqpllsntl aelyvrehlr eqsrkppnpt |
| 481 | ppppgasana sverikttss iefarlqfty nhiqrhvndm lgrvaiawce lqnheltlwn |
| 541 | earklnpnai asatvgrrvs armlgdvmav stcvpvaadn vivqnsmris srpgacysrp |
| 601 | lvsfryedqg plvegqlgen nelrltrdai epctvghrry ftfgggyvyf eeyayshqls |
| 661 | radittvstf idlnitmled hefvplevyt rheikdsgll dytevqrrnq lhdlrfadid |
| 721 | tvihadanaa mfaglgaffe gmgdlgravg kvvmgivggv vsaysgvssf msnpfgalav |
| 781 | gllvlaglaa affafryvmr lqsnpmkaly plttkelknp tnpdasgege eggdfdeakl |
| 841 | aearemirym alvsamerte hkakkkgtsa llsakvtdmv mrkrrntnyt qvpnkdgdad |
| 901 | eddl |
| UL29/ICP8 | |
| (SEQ ID NO: 52) | |
| 1 | metkpktatt ikvppgplgy vyaracpseg iellallsar sgdsdvavap lvvgltvesg |
| 61 | feanvavvvg srttglggta vslkltpshy sssvyvfhgg rhldpstqap nltrlcerar |
| 121 | rhfgfsdytp rpgdlkhett gealcerlgl dpdrallylv vtegfkeavc inntflhlgg |
| 181 | sdkvtiggae vhripvyplq lfmpdfsrvi aepfnanhrs igekftyplp ffnrplnrll |
| 241 | feavvgpaav alrcrnvdav araaahlafd enhegaalpa ditftafeas qgktprggrd |
| 301 | gggkgaaggf eqrlasvmag daalalesiv smavfdeppt disawplfeg qdtaaarana |
| 361 | vgaylaraag lvgamvfstn salhltevdd agpadpkdhs kpsfyrfflv pgthvaanpq |
| 421 | vdreghvvpg fegrptaplv ggtqefageh lamlcgfspa llakmlfyle rcdgavivgr |
| 481 | qemdvfryva dsnqtdvpcn lctfdtrhac vhttlmrlra rhpkfasaar gaigvfgtmn |
| 541 | smysdcdvlg nyaafsalkr adgsetarti mqetyraate rvmaeletlq yvdqavptam |
| 601 | grletiitnr ealhtvvnnv rqvvdreveq lmrnlvegrn fkfrdglgea nhamsltldp |
| 661 | yacgpcpllq llgrrsnlav yqdlalsqch gvfagqsveg rnfrnqfqpv lrrrvmdmfn |
| 721 | ngflsaktlt valsegaaic apsltagqta paessfegdv arvtlgfpke lrvksrvlfa |
| 781 | gasanaseaa karvaslqsa yqkpdkrvdi llgplgfllk qfhaaifpng kppgsnqpnp |
| 841 | qwfwtalqrn qlparllsre dietiafikk fsldygainf inlapnnvse lamyymanqi |
| 901 | lrycdhstyf intltaiiag srrppsvqaa aawsaqggag leagaralmd avdahpgawt |
| 961 | smfascnllr pvmaarpmvv lglsiskyyg magndrvfqa gnwaslmggk nacpllifdr |
| 1021 | trkfvlacpr agfvcaassl gggahesslc eqlrgiiseg gaavassvfv atvkslgprt |
| 1081 | qqlqiedwla lledeylsee mmeltarale rgngewstda alevaheaea lvsqlgnage |
| 1141 | vfnfgdfgce ddnatpfggp gapgpafagr krafhgddpf gegppdkkgd ltldml |
| UL31 | |
| (SEQ ID NO: 53) | |
| 1 | mydtdphrrg srpgpyhgke rrrsrssaag gtlgvvrras rkslpphark qelclherqr |
| 61 | yrglfaalaq tpseeiaivr slsvplvktt pvslpfcldq tvadncltls gmgyylgigg |
| 121 | ccpacnagdg rfaatsreal ilafvqqint ifehraflas lvvladrhna plqdllagil |
| 181 | gqpelffvht ilrgggacdp rllfypdpty gghmlyvifp gtsahlhyrl idrmltacpg |
| 241 | yrfvahvwqs tfvlvvrrna ekptdaeipt vsaadiyckm rdisfdgglm leyqrlyatf |
| 301 | defppp |
| UL37/ICP32 | |
| (SEQ ID NO: 54) | |
| 1 | madrglpsea pvvttspagp psdgpmqrll aslaglrqpp tptaetanga ddpaflatak |
| 61 | lraamaafll sgtaiapada rdcwrplleh lcalhrahgl petallaenl pgllvhrlvv |
| 121 | alpeapdqaf remevikdti lavtgsdtsh aldsaglrta aalgpvrvrq cavewidrwq |
| 181 | tvtksclams prtsiealge tslkmapvpl gqpsanlttp aysllfpapf vqeglrflal |
| 241 | vsnrvtlfsa hlqriddatl tpltralftl alvdeylttp ergavvpppl laqfqhtvre |
| 301 | idpaimippl eankmvrsre evrvstalsr vsprsacapp gtlmarvrtd vavfdpdvpf |
| 361 | lsssalavfq payssllqlg eqpsagaqqr llallqqtwt liqntnspsv vintlidagf |
| 421 | tpshcthyls alegflaagv partptghgl gevqqlfgci alagsnvfgl areygyyany |
| 481 | vktfrrvqga sehthgrlce avglsggvls qtlarimgpa vptehlaslr ralvgefeta |
| 541 | errfssgqps llretaliwi dvygqthwdi tpttpatpls allpvgqpsh apsvhlaaat |
| 601 | qirfpalegi hpnvladpgf vpyvlalvvg dalratcsaa ylprpvefal rvlawardfg |
| 661 | lgylptvegh rtklgalitl lepaargglg ptmqmadnie qllrelyvis rgaveqlrpl |
| 721 | vqlqpppppe vgtslllism yalaargvlq dlaeradpli rqledaivll rlhmrtlsaf |
| 781 | fecrfesdgr rlyavvgdtp drlgpwppea mgdavsqycs myhdakralv aslaslrsvi |
| 841 | tettahlgvc delaaqvshe dnvlavvrre ihgflsvvsg iharasklls gdqvpgfcfm |
| 901 | gqflarwrrl sacyqaaraa agpepvaefv qelhdtwkgl qteravvvap lvssadqraa |
| 961 | airevmahap edappqspaa drvvltsrrd lgawgdyslg plgqttavpd svdlsrqgla |
| 1021 | vtlsmdwllm nellrvtdgv frasafrpla gpesprdlev rdagnslpap mpmdaqkpea |
| 1081 | yghgprqadr egaphsntpv eddemipedt vapptdlplt syq |
| UL39/ICP6/10 | |
| (SEQ ID NO: 55) | |
| 1 | masrpaassp vearapvggq eaggpsaatq geaagaplah ghhvycqrvn gvmvlsdktp |
| 61 | gsasyrisdn nfvqcgsnct miidgdvvrg rpqdpgaaas papfvavtni gagsdggtav |
| 121 | vafggtprrs agtstgtqta dvptealggp pppprftlgg gccscrdtrr rsavfggegd |
| 181 | pvgpaefvsd drssdsdsdd sedtdsetls hassdvsgga tyddaldsds ssddslqidg |
| 241 | pvcrpwsndt apldvcpgtp gpgadaggps avdphaptpe agaglaadpa varddaegls |
| 301 | dprprlgtgt aypvpleltp enaeavarfl gdavnrepal mleyfcrcar eetkrvpprt |
| 361 | fgspprlted dfgllnyalv emqrlcldvp pvppnaympy ylreyvtrlv ngfkplvsrs |
| 421 | arlyrilgvl vhlrirtrea sfeewlrske valdfglter lreheaqlvi laqaldhydc |
| 481 | lihstphtiv erglqsalky eefylkrfgg hymesvfqmy triagflacr atrgmrhial |
| 541 | gregswwemf kfffhrlydh qivpstpaml nlgtrnyyts scylvnpqat tnkatlrait |
| 601 | snvsailarn ggiglcvqaf ndsgpgtasv mpalkvldsl vaahnkesar ptgacvylep |
| 661 | whtdvravlr mkgvlageea qrcdnifsal wmpdlffkrl irhldgeknv twtlfdrdts |
| 721 | msladfhgee feklyqhlev mgfgeqipiq elaygivrsa attgspfvmf kdavnrhyiy |
| 781 | dtqgaaiags nlcteivhpa skrssgvcnl gsvnlarcvs rqtfdfgrlr davqacvlmv |
| 841 | nimidstlqp tpqctrgndn lrsmgigmqg lhtaclklgl dlesaefqdl nkhiaevmll |
| 901 | samktsnalc vrgarpfnhf krsmyragrf hwerfpdarp ryegewemlr qsmmkhglrn |
| 961 | sqfvalmpta asagisdvse gfaplftnlf skvtrdgetl rpntlllkel ertfsgkrll |
| 1021 | evmdsldakq wsvaqalpcl epthplrrfk tafdydqkll idlcadrapy vdhsqsmtly |
| 1081 | vtekadgtlp astlvrllvh aykrglktgm yyckvrkatn sgvfggddni vcmscal |
| UL40 | |
| (SEQ ID NO: 56) | |
| 1 | mdsaapalsp altaltdqsa tadlaiqipk cpdperyfyt sqcpdinhlr slsilnrwle |
| 61 | telvfvgdee dvsklsegel sfyrflfafl saaddlvten lgglsglfeq kdilhyyveq |
| 121 | ecievvhsrv yniiqlvlfh nndqarreyv agtinhpair akvdwlearv recasvpekf |
| 181 | ilmiliegif faasfaaiay lrtnnllrvt cqsndlisrd eavhttascy iynnylggha |
| 241 | kpppdrvygl frqaveieig firsqaptds hilspaalaa ienyvrfsad rllglihmkp |
| 301 | lfsapppdas fplslmstdk htnffecrst syagavvndl |
| UL41/vhs | |
| (SEQ ID NO: 57) | |
| 1 | mglfgmmkfa hthhlvkrrg lgapagyftp iavdlwnvmy tivvkyqrry psydreaitl |
| 61 | hclcrllkvf tqkslfpifv tdrgvncmep vvfgakaila rttaqcrtde easdvdaspp |
| 121 | pspitdsrps safsnmrrrg tslasgtrgt agsgaalpsa apskpalrla hlfcirvlra |
| 181 | lgyayinsgq leaddacanl yhtntvayvy ttdtdlllmg cdivldisac yiptincrdi |
| 241 | lkyfkmsypq flalfvrcht dlhpnntyas vedvlrechw tppsrsqtrr airrehtssr |
| 301 | stetrpplpp aaggtetrvs wteiltqqia ggyeddedlp ldprdvtggh pgprssssei |
| 361 | ltppelvqvp naqlleehrs yvanprrhvi hdapesldwl pdpmtitelv ehryikyvis |
| 421 | ligpkergpw tllkrlpiyq dirdenlars ivtrhitapd iadrfleqlr tqapppafyk |
| 481 | dvlakfwde |
| UL46/VP11/12 | |
| (SEQ ID NO: 58) | |
| 1 | mqrrtrgass lrlarcltpa nlirgdnagv perrifggcl lptpegllsa avgalrqrsd |
| 61 | daqpafltct drsvrlaarq hntvpesliv dglasdphye yirhyasaat qalgevelpg |
| 121 | gqlsrailtq ywkylqtvvp sgldvpedpv gdcdpslhvl lrptlapkll artpfksgav |
| 181 | aakyaatvag lrdalhriqq ymffmrpadp srpstdtalr lnellayvsv lyrwaswmlw |
| 241 | ttdkhvchrl spsnrrflpl ggspeapaet farhldrgps gttgsmqcma lraaysdvlg |
| 301 | hltrlanlwq tgkrsggtyg tvdtvvstve vlsivhhhaq yiinatltgy gvwatdslnn |
| 361 | eylraavdsq erfcrttapl fptmtapswa rmelsikawf gaalaadllr ngapslhyes |
| 421 | ilrlvasrrt twsagpppdd masgpgghra gggtcrekiq rarrdneppp lprprlhstp |
| 481 | astrrfrrrr adgagpplpd andpvaeppa aatqpatyyt hmgevpprlp arnvagpdrr |
| 541 | ppaatcpllv rraslgsldr prvwgpapeg epdqmeatyl taddddddar rkathaasar |
| 601 | erhapyedde siyetvsedg grvyeeipwm rvyenvcvnt anaapaspyi eaenplydwg |
| 661 | gsalfsppgr tgppppplsp spvlarhran altndgptnv aalsalltkl kregrrsr |
| UL47/VP13/14 | |
| (SEQ ID NO: 59) | |
| 1 | msarepagrr rrastrpras pvadepagdg vgfmgylrav frgdddsele aleemagdep |
| 61 | pvrrrregpr arrrraseap ptshrrasrq rpgpdaarsq svrgrldddd evprgppqar |
| 121 | qggylgpvda railgrvggs rvapsplfle elqyeeddyp eavgpedggg arsppkvevl |
| 181 | egrvpgpelr aafpldrlap qvavwdesvr salalghpag fypcpdsafg lsrvgvmhfa |
| 241 | spdnpavffr qtlqqgeala wyitgdgild ltdrrtktsp aqamsflada vvrlaingwv |
| 301 | cgtrlhaear gsdlddraae lrrqfaslta lrpvgaaavp llsagglvsp qsgpdaavfr |
| 361 | sslgsllywp gvralldrdc rvaaryagrm tylatgalla rfnpdavrcv ltreaaflgr |
| 421 | vldvlavmae qtvqwlsvvv garlhphvhh pafadvaree lfralplgsp avvgaeheal |
| 481 | gdtaarrlla nsglnavlga avyalhtala tvtlkyarac gdahrrrdda aatrailaag |
| 541 | lvlqrllgfa dtvvacvtla afdggftape vgtytplrya cvlratqply arttpakfwa |
| 601 | dvraaaehvd lrpassapra pvsgtadpaf llkdlepfpp apvsggsvlg prvrvvdims |
| 661 | qfrkllmgde gaaalrahvs grratglggp prp |
| UL48/VP16/ICP25 | |
| (SEQ ID NO: 60) | |
| 1 | mdllvdelfa dmnadgaspp pprpaggpkn tpaapplyat grlsqaqlmp sppmpvppaa |
| 61 | lfnrllddlg fsagpalctm ldtwnedlfs alptnadlyr eckflstlps dvvewgdayv |
| 121 | pertqidira hgdvafptlp atrdglglyy ealsrffhae lrareesyrt vlanfcsaly |
| 181 | rylrasvrql hrqahmrgrd rdlgemlrat iadryyreta rlarvlflh1 ylfltreilw |
| 241 | aayaeqmmrp dlfdclccdl eswrqlaglf qpfmfvngal tvrgvpiear rlrelnhire |
| 301 | hlnlplvrsa ateepgaplt tpptlhgnqa rasgyfmvli rakldsyssf ttspseavmr |
| 361 | ehaysrartk nnygstiegl ldlpdddape eaglaaprls flpaghtrrl stapptdvsl |
| 421 | gdelhldged vamahadald dfdldmlgdg dspgpgftph dsapygaldm adfefeqmft |
| 481 | dalgideygg |
| UL49/VP22 | |
| (SEQ ID NO: 61) | |
| 1 | mtsrrsvksg prevprdeye dlyytpssgm aspdsppdts rrgalqtrsr qrgevrfvqy |
| 61 | desdyalygg ssseddehpe vprtrrpvsg avlsgpgpar appppagsgg agrtpttapr |
| 121 | aprtqrvatk apaapaaett rgrksaqpes aalpdapast aptrsktpaq glarklhfst |
| 181 | appnpdapwt prvagfnkrv fcaavgrlaa mharmaavql wdmsrprtde dlnellgitt |
| 241 | irvtvcegkn llqranelvn pdvvqdvdaa tatrgrsaas rpterprapa rsasrprrpv |
| 301 | e |
| UL53/Glycoprotein K | |
| (SEQ ID NO: 62) | |
| 1 | mlavrslqhl stvvlitayg lvlvwytvfg asplhrciyv vrptgtnndt alvwmkmnqt |
| 61 | llflgapthp pnggwrnhah isyanliagr vvpfqvppda mnrrimnvhe avncletlwy |
| 121 | trvrlvvvgw flylafvalh qrrcmfgvvs pahkmvapat yllnytgriv ssvflqypyt |
| 181 | kitrllcels vqrqnlvqlf etdpvtflyh rpaigvivgc elivrfvavg livgtafisr |
| 241 | gacaityplf ltittwcfvs tigltelyci lrrgpapkna dkaaapgrsk glsgvcgrcc |
| 301 | siilsgiamr lcyiavvagv vlvalhyeqe iqrrlfdv |
| UL54ICP27 | |
| (SEQ ID NO: 63) | |
| 1 | matdidmlid lgldlsdsdl dedppepaes rrddlesdss gecsssdedm edphgedgpe |
| 61 | pildaarpav rpsrpedpgv pstqtprpte rqgpndpqpa phsvwsrlga rrpscspeqh |
| 121 | ggkvarlqpp ptkaqpargg rrgrrrgrgr ggpgaadgls dprrraprtn rnpggprpga |
| 181 | gwtdgpgaph geawrgseqp dppggqrtrg vrqappplmt laiapppadp rapaperkap |
| 241 | aadtidattr lvlrsisera avdrisesfg rsaqvmhdpf ggqpfpaans pwapvlagqg |
| 301 | gpfdaetrrv swetlvahgp slyrtfagnp raastakamr dcvlrqenfi ealasadetl |
| 361 | awckmcihhn lplrpqdpii gttaavldnl atrlrpflqc ylkarglcgl delcsrrrla |
| 421 | dikdiasfvf vilarlanrv ergvaeidya tlgvgvgekm hfylpgacma glieildthr |
| 481 | qecssrvcel tashivappy vhgkyfycns if |
| US1/ICP22 | |
| (SEQ ID NO: 64) | |
| 1 | madispgafa pcvkarrpal rspplgtrkr krpsrplsse sevesdtale sevesetasd |
| 61 | stesgdqdea priggrrapr rlggrffldm saesttgtet dasvsddpdd tsdwsyddip |
| 121 | prpkrarvnl rltsspdrrd gvifpkmgrv rstretqpra ptpsapspna mlrrsvrqaq |
| 181 | rrssarwtpd lgymrqcinq lfrvlrvard phgsanrlrh lirdcylmgy crarlaprtw |
| 241 | crllqvsggt wgmhlrntir evearfdata epvcklpcle trrygpecdl snleihlsat |
| 301 | sddeisdatd leaagsdhtl asqsdtedap spvtletpep rgslavrled efgefdwtpq |
| 361 | egsqpwlsav vadtssverp gpsdsgagra aedrkcldgc rkmrfstacp ypcsdtflrp |
| US7/Glycoprotein I | |
| (SEQ ID NO: 65) | |
| 1 | mpcrplqglv lvglwvcats lvvrgptvsl vsnsfvdaga lgpdgvveed llilgelrfv |
| 61 | gdqvphttyy dggvelwhyp mghkcprvvh vvtvtacprr pavafalcra tdsthspayp |
| 121 | tlelnlaqqp llrvqratrd yagvyvlrvw vgdapnaslf vlgmaiaaeg tlayngsayg |
| 181 | scdpkllpss aprlapasvy qpapnqastp stttstpstt ipapsttipa pqasttpfpt |
| 241 | gdpkpqppgv nheppsnatr atrdsryalt vtqiiqiaip asiialvflg scicfihrcq |
| 301 | rryrrsrrpi yspqmptgis cavneaamar lgaelkshps tppksrrrss rtpmpsltai |
| 361 | aeesepagaa glptppvdpt tptptppllv |
| RL2/ICP0 | |
| (SEQ ID NO: 66) | |
| 1 | meprpgastr rpegrpqrep apdvwvfpcd rdlpdssdse aetevggrgd adhhdddsas |
| 61 | eadstdtelf etgllgpqgv dggaysggsp preedpgscg gappredggs degdvcavct |
| 121 | deiaphlrcd tfpcmhrfci pcmktwmqlr ntcplcnakl vylivgvtps gsfstipivn |
| 181 | dpqtrmeaee avragtavdf iwtgnqrfap ryltlgghtv ralspthpep ttdeddddld |
| 241 | dadyvppapr rtprapprrg aaappvtgga shaapqpaaa rtappsapig phgssntntt |
| 301 | tnssggggsr qsraaaprga sgpsggvgvg vgvveaeagr prgrtgplvn rpaplannrd |
| 361 | pivisdsppa sphrppaapm pgsaprpgpp asaaasgpar praavapcvr apppgpgpra |
| 421 | papgaepaar padarrvpqs hsslaqaanq eqslcrarat vargsggpgv egghgpsrga |
| 481 | apsgaaplps aasveqeaav rprkrrgsgq enpspqstrp plapagakra athppsdsgp |
| 541 | ggrgqggpgt pltssaasas sssassssap tpagaassaa gaasssasas sggavgalgg |
| 601 | rqeetslgpr aasgprgprk carktrhaet sgavpagglt rylpisgvss vvalspyvnk |
| 661 | titgdclpil dmetgnigay vvlvdqtgnm atrlraavpg wsrrtllpet agnhvmppey |
| 721 | ptapasewns lwmtpvgnml fdqgtlvgal dfrslrsrhp wsgeqgastr degkq |
| RS1/ICP4 | |
| (SEQ ID NO: 67) | |
| 1 | masenkqrpg spgptdgppp tpspdrderg algwgaetee ggddpdhdpd hphdlddarr |
| 61 | dgrapaagtd agedagdavs prqlallasm veeavrtipt pdpaaspprt pafraddddg |
| 121 | deyddaadaa gdrapargre reaplrgayp dptdrlsprp paqpprrrrh grwrpsasst |
| 181 | ssdsgsssss sasssssssd ededddgnda adharearav grgpssaapa apgrtppppg |
| 241 | ppplseaapk praaartpaa sagrierrra raavagrdat grftagqprr veldadatsg |
| 301 | afyaryrdgy vsgepwpgag ppppgrvlyg glgdsrpglw gapeaeearr rfeasgapaa |
| 361 | vwapelgdaa qqyalitrll ytpdaeamgw lqnprvvpgd valdqacfri sgaarnsssf |
| 421 | itgsvaravp hlgyamaagr fgwglahaaa avamsrrydr aqkgflltsl rrayapllar |
| 481 | enaaltgaag spgagaddeg vaavaaaapg eravpagyga agilaalgrl saapaspagg |
| 541 | ddpdaarhad adddagrraq agrvavecla acrgileala egfdgdlaav pglagarpas |
| 601 | pprpegpagp aspppphada prlrawlrel rfvrdalvlm rlrgdlrvag gseaavaavr |
| 661 | avslvagalg palprdprlp ssaaaaaadl lfdnqslrpl laaaasapda adalaaaaas |
| 721 | aapregrkrk spgparppgg ggprppktkk sgadapgsda raplpapapp stppgpepap |
| 781 | aqpaapraaa aqarprpvav srrpaegpdp lggwrrqppg pshtaapaaa aleaycspra |
| 841 | vaeltdhplf pvpwrpalmf dpralasiaa rcagpapaaq aacgggdddd nphphgaagg |
| 901 | rlfgplrasg plrrmaawmr qipdpedvrv vvlysplpge dlagggasgg ppewsaergg |
| 961 | lscllaalan rlcgpdtaaw agnwtgapdv salgaqgvll lstrdlafag aveflgllas |
| 1021 | agdrrlivvn tvracdwpad gpavsrqhay lacellpavq cavrwpaard lrrtvlasgr |
| 1081 | vfgpgvfarv eaaharlypd applrlcrgg nvryrvrtrf gpdtpvpmsp reyrravlpa |
| 1141 | ldgraaasgt tdamapgapd fceeeahsha acarwglgap lrpvyvalgr eavragparw |
| 1201 | rgprrdfcar allepdddap plvlrgdddg pgalppappg irwasatgrs gtvlaaagav |
| 1261 | evlgaeagla tpprrevvdw egawdeddgg afegdgvl |

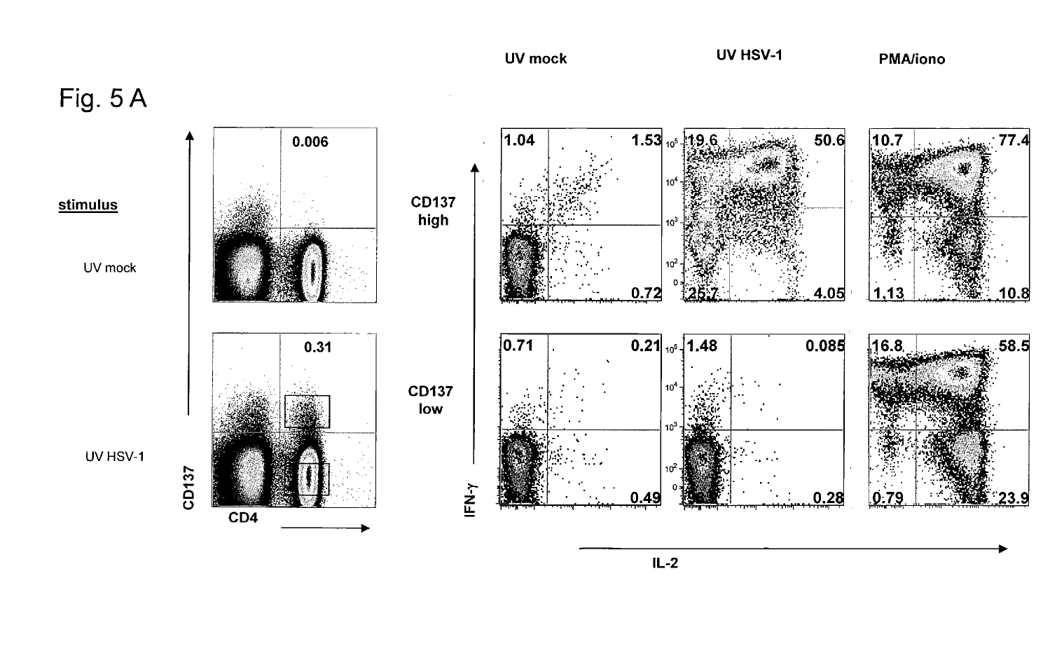

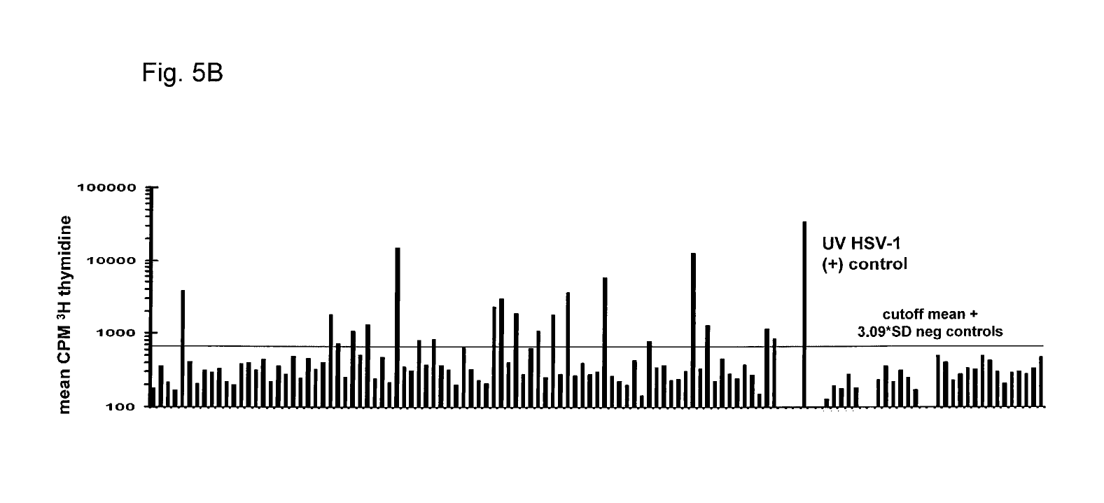

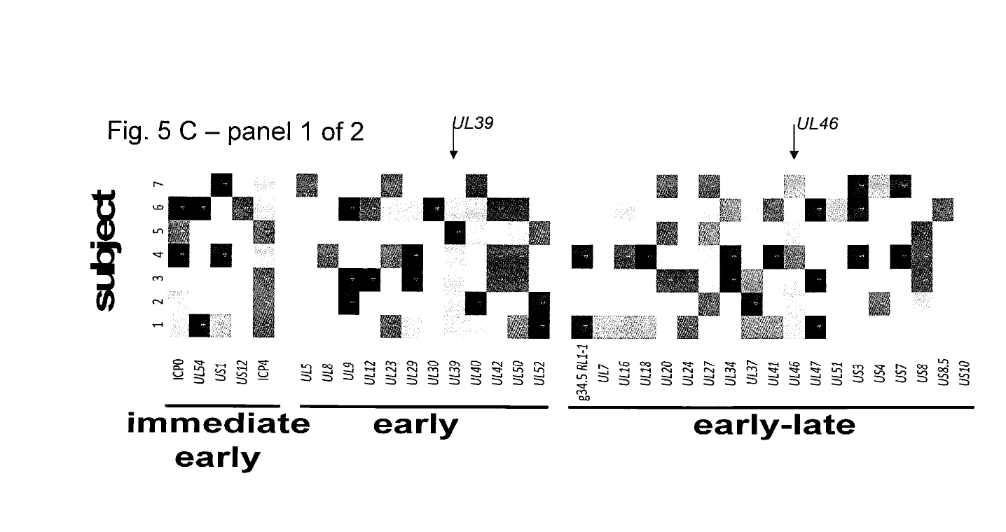

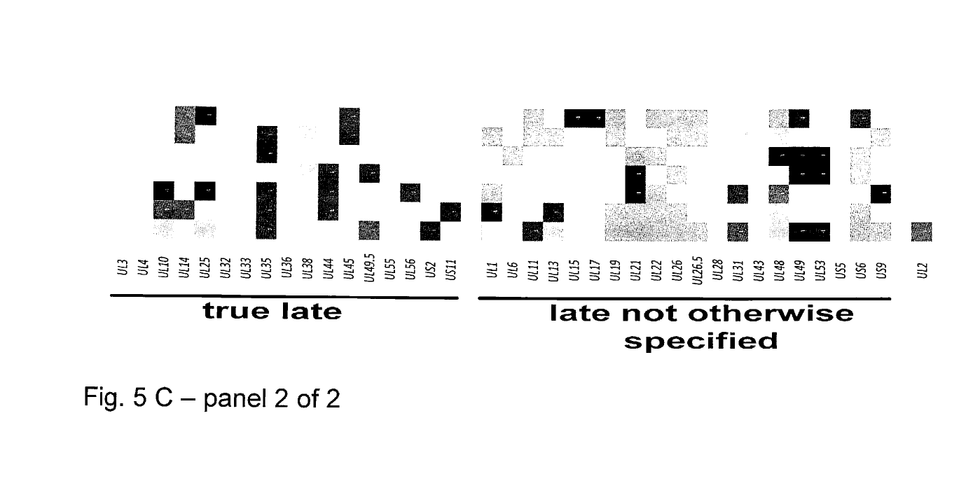

As indicated in FIG. 5C, some antigens of the invention elicit primarily CD4+ T cell reactions in HSV-infected subjects, while others elicit primarily CD8+ T cells reactions in HSV-infected subjects. Some HSV-1 antigens elicit both CD4 and CD8 T-cells in many subjects, and these antigens eliciting coordinated immune responses are considered especially valuable. Thus, in one embodiment, the HSV polypeptide is one that elicits both CD4 and CD8 responses. In one embodiment, the HSV polypeptide comprises multiple epitopes, as set forth in Table 4, wherein the epitopes may be from the same HSV protein or from more than one HSV protein. The HSV polypeptide comprising one or more epitopes of the invention can comprise a fragment of a full-length HSV protein, or the full-length HSV protein. In some embodiments, multiple HSV polypeptides are provided together within a single composition, within a kit, or within a larger polypeptide. In one embodiment, the invention provides a multi-epitopic or multi-valent vaccine.

The embodiments comprising multiple HSV polypeptides include any combination of two or more of the epitopes listed in Table 4 or the corresponding full-length proteins, and, optionally, additional HSV polypeptides of HSV-1 and/or HSV-2, including those described in United States patent publication number US-2010-0203073-A1, published on Aug. 12, 2010, namely, VP16, gK or gL, or fragments thereof that include amino acids 64-160, 90-99, 141-240, 187-199, 191-203, 215-227, 218-320, 219-230, 381-490, 479-489, 479-488, 480-488 or 477-490 of VP16 (UL48); 201-209 of glycoprotein K (UL53); or 66-74 of glycoprotein L (UL1).

In one embodiment, the HSV polypeptide comprises UL1, UL13, UL21, UL25, UL26, UL27, UL29, UL31, UL37, UL39, UL40, UL41, UL46, UL47, UL48, UL49, UL53, UL54, US1, US7, ICP0, ICP4, or any combination of two or more of the preceding polypeptides. The polypeptide can include the full-length of one or more of the HSV proteins, or a portion that includes one or more epitopes as described herein. In another embodiment, the HSV polypeptide comprises one or more epitopes selected from the group consisting of each of the peptides listed in Table 4.

In another embodiment, the HSV polypeptide comprises one or more epitopes that have not been previously described as CD8 epitopes with the same proven or probable HLA restriction using PBMC from HSV-2-infected persons and HSV-2 peptides. For example, the HSV polypeptide comprises one or more epitopes selected from the group consisting of: amino acids 66-74 of UL1 (LIDGIFLRY; SEQ ID NO: 1), amino acids 259-268 of UL41 (HTDLHPNNTY; SEQ ID NO: 3), amino acids 360-368 of UL47 (RSSLGSLLY; SEQ ID NO: 5), amino acids 566-574 of UL47 (FTAPEVGTY; SEQ ID NO: 6), amino acids 90-99 of UL48 (SALPTNADLY; SEQ ID NO: 7), amino acids 479-488 of UL48 (FTDALGIDEY; SEQ ID NO: 8), amino acids 201-209 of UL53 (ETDPVTFLY; SEQ ID NO: 9), amino acids 389-397 of UL13 (TLLELVVSV; SEQ ID NO: 10), amino acids 280-288 of UL27 (SVYPYDEFV; SEQ ID NO: 12), amino acids 425-433 of UL39 (RILGVLVHL; SEQ ID NO: 14), amino acids 184-192 of UL40 (ILIEGIFFA; SEQ ID NO: 15), amino acids 286-294 of UL47 (FLADAVVRL; SEQ ID NO: 16), amino acids 374-382 of UL47 (ALLDRDCRV; SEQ ID NO: 17), amino acids 545-553 of UL47 (RLLGFADTV; SEQ ID NO: 18), amino acids 162-170 of UL21 (VYTPSPYVF; SEQ ID NO: 19), amino acids 292-300 of UL31 (EYQRLYATF; SEQ ID NO: 20), amino acids 221-230 of UL37 (AYSLLFPAPF; SEQ ID NO: 21), amino acids 640-648 of UL37 (AYLPRPVEF; SEQ ID NO: 22), amino acids 226-234 of UL46 (AYVSVLYRW; SEQ ID NO: 23), amino acids 504-512 of UL54 (KYFYCNSLF; SEQ ID NO: 24), amino acids 1097-1106 of ICP4 (LYPDAPPLRL; SEQ ID NO: 25), amino acids 170-179 of UL25 (SSGVVFGTWY; SEQ ID NO: 26), amino acids 235-243 of UL25 (AVLCLYLLY; SEQ ID NO: 27), amino acids 22-30 of UL26 (YVAGFLALY; SEQ ID NO: 28), amino acids 326-334 of UL26 (YLWIPASHY; SEQ ID NO: 29), amino acids 295-303 of UL27 (VYMSPFYGY; SEQ ID NO: 30), amino acids 641-649 of UL27 (FTFGGGYVY; SEQ ID NO: 31), amino acids 460-468 of UL29 (ALLAKMLFY; SEQ ID NO: 32), amino acids 895-903 of UL29 (YMANQILRY; SEQ ID NO: 33), amino acids 93-101 of UL46 (LASDPHYEY; SEQ ID NO: 34), amino acids 126-134 of UL46 (AILTQYWKY; SEQ ID NO: 35), amino acids 224-232 of UL46 (LLAYVSVLY; SEQ ID NO: 36), amino acids 333-341 of UL46 SIVHHHAQY (SEQ ID NO: 37), amino acids 508-516 of UL47 ALATVTLKY (SEQ ID NO: 38), amino acids 382-390 of UL21 VPRPDDPVL (SEQ ID NO: 40), amino acids 281-290 of UL49 RPTERPRAPA (SEQ ID NO: 41), amino acids 70-78 of US1 APRIGGRRA (SEQ ID NO: 42), amino acids 22-30 of US7 VVRGPTVSL (SEQ ID NO: 43), amino acids 97-105 of US7 CPRRPAVAF (SEQ ID NO: 44), and amino acids 195-203 of US7 APASVYQPA (SEQ ID NO: 45).

In another embodiment, the HSV polypeptide comprises one or more type-specific HSV-1 (versus HSV-2) epitopes as identified in Table 4. In an alternative embodiment, the HSV polypeptide comprises one or more type-common (HSV-1 and HSV-2) epitopes as identified in Table 4. In a further embodiment, the HSV polypeptide comprises a combination of type-common and type-specific epitopes. In another embodiment, the HSV polypeptide comprises one or more of the epitopes identified as recognized by T cells of the human trigeminal ganglia, including epitopes of VP16 (gene UL48), immediate early proteins UL39 and ICP0, and late glycoproteins K and L, alone or in combination with one or more of the polypeptides disclosed herein. In one embodiment, the HSV polypeptide comprises epitopes of VP16/UL48, UL39 and/or ICP0.

In some embodiments, the selection of a combination of epitopes and/or antigens to be included within a single composition and/or polypeptide is guided by optimization of population coverage with respect to HLA alleles. For example, each epitope restricted by HLA allele A*0201 will cover 40-50% of most ethnic groups. By adding epitopes restricted by A*0101 (20%), A*2402 (˜5-25%), B*0702 (10-15%), and A*29 (5-10%), one can, in the aggregate, cover more people. In one embodiment, the HSV polypeptide comprises one or more of the epitopes identified in Table 4 as associated with HLA allele A*0101. In another embodiment, the HSV polypeptide comprises one or more of the epitopes identified in Table 4 as associated with HLA allele A*0201. In another embodiment, the HSV polypeptide comprises one or more of the epitopes identified in Table 4 as associated with HLA allele A*2402. In another embodiment, the HSV polypeptide comprises one or more of the epitopes identified in Table 4 as associated with HLA allele A*2902. In another embodiment, the HSV polypeptide comprises one or more of the epitopes identified in Table 4 as associated with HLA allele B*0702. In a further embodiment, the HSV polypeptide comprises epitopes identified in Table 4 as associated with 2, 3, 4 or all 5 of the HLA alleles, A*0101, A*0201, A*2402, A*2902, and B*0702. As is understood by those skilled in the art, these HLA alleles, or HLA alleles that are biologically expected to bind to peptide epitopes restricted by these HLA alleles, cover 80-90% of the human population in most major ethnic and racial groups.

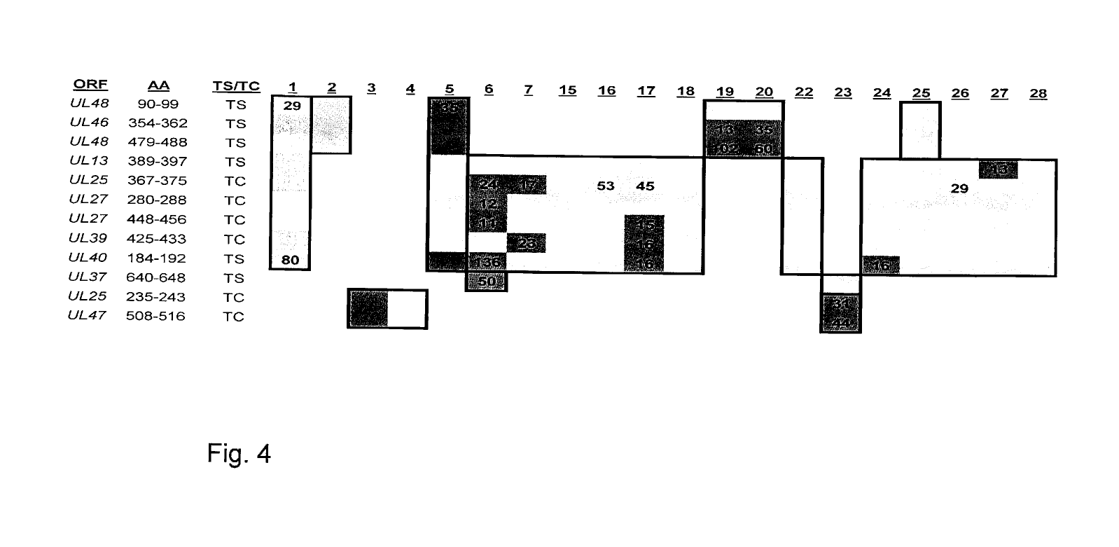

In one embodiment, the HSV polypeptide comprises all of UL1, UL13, UL21, UL25, UL26, UL27, UL29, UL31, UL37, UL39, UL40, UL41, UL46, UL47, UL48, UL49, UL53, UL54, US1, US7, ICP0, and ICP4, not necessarily in that order. In another embodiment, the HSV polypeptide comprises all of the epitopes listed in Table 4, not necessarily in the order listed. In one embodiment, the invention provides UL39 and UL48, optionally in combination with UL46 and/or UL40, as full-length proteins and/or as fragments thereof that include one or more epitopes identified in Table 4. In one embodiment, the invention provides UL25, UL39 and UL47, as full-length proteins and/or as fragments thereof that include one or more epitopes identified in Table 4. In one embodiment, the invention provides UL25 and UL47, as full-length proteins and/or as fragments thereof that include one or more epitopes identified in Table 4. In one embodiment, the invention provides UL25 and UL39, as full-length proteins and/or as fragments thereof that include one or more epitopes identified in Table 4. In one embodiment, the invention provides UL39 and UL47, as full-length proteins and/or as fragments thereof that include one or more epitopes identified in Table 4. In one embodiment, the invention provides UL46, UL47, UL49, and/or UL21, as full-length proteins and/or as fragments thereof that include one or more epitopes identified in Table 4. In one embodiment, the invention provides UL39 and/or UL46, as full-length proteins and/or as fragments thereof that include one or more epitopes identified in Table 4. The selection of particular combinations of antigens and/or epitopes can be guided by the data described in Example 1, including that presented in FIGS. 4 and 5. For example, antigens that exhibit desirable characteristics per FIG. 4 and/or those that include multiple immunogenic epitopes can be combined in a single composition and/or polypeptide.

In each of the embodiments described herein, the HSV polypeptide, or epitope thereof, may be present alone or in combination with other epitopes listed in Table 4, or with other epitopes of HSV-1 or HSV-2; as a single contiguous polypeptide, or as a composition or kit comprising multiple polypeptides. For embodiments in which the epitopes are present as a single continuous polypeptide, those skilled in the art will appreciate that the epitopes may be adjacent to one another, or present as epitopes separated by short linker sequences selected to enhance epitope release during antigen processing in cells. For example, in one embodiment, the polypeptide consists of one or more of the HSV-1 proteins selected from the group consisting of UL1, UL13, UL21, UL25, UL26, UL27, UL29, UL31, UL37, UL39, UL40, UL41, UL46, UL47, UL48, UL49, UL53, UL54, US1, US7, ICP0, and ICP4, optionally, up to 100 amino acid residues of linker sequence between said proteins. In another example, the polypeptide consists of one or more of the epitopes listed in Table 4 and, optionally, up to 100 amino acid residues of linker sequence between said eptiopes. Typically, a linker comprises up to 10, up to 50, or up to 100 amino acid residues. One skilled in the art can appreciate the appropriate options for selecting a linker sequence.

A fragment of the invention consists of less than the complete amino acid sequence of the corresponding protein, but includes the recited epitope or antigenic region. As is understood in the art and confirmed by assays conducted using fragments of widely varying lengths, additional sequence beyond the recited epitope can be included without hindering the immunological response. A fragment of the invention can be as few as 8 amino acids in length, or can encompass 10%, 20%, 30%, 40%, 50%, 60%, 70%, 80%, 90% or 95% of the full length of the protein.

The optimal length for the polypeptide of the invention will vary with the context and objective of the particular use, as is understood by those in the art. In some vaccine contexts, a full-length protein or large portion of the protein (e.g., up to 100 amino acids, 150 amino acids, 200 amino acids, 250 amino acids or more) provides optimal immunological stimulation, while in others, a short polypeptide (e.g., less than 50 amino acids, 40 amino acids, 30 amino acids, 20 amino acids, 15 amino acids or fewer) comprising the minimal epitope and/or a small region of adjacent sequence facilitates delivery and/or eases formation of a fusion protein or other means of combining the polypeptide with another molecule or adjuvant.

A polypeptide for use in a composition of the invention comprises a HSV polypeptide that contains an epitope or minimal stretch of amino acids sufficient to elicit an immune response. These polypeptides typically consist of such an epitope and, optionally, adjacent sequence. Those skilled in the art are aware that the HSV epitope can still be immunologically effective with a small portion of adjacent HSV or other amino acid sequence present. Accordingly, a typical minimal polypeptide of the invention will consist essentially of the recited HSV epitope and have a total length of up to 15, 20, 25 or 30 amino acids.

In general, polypeptides (including fusion proteins) and polynucleotides as described herein are isolated. An “isolated” polypeptide or polynucleotide is one that is removed from its original environment. For example, a naturally occurring protein is isolated if it is separated from some or all of the coexisting materials in the natural system. An isolated HSV polypeptide of the invention is one that has been isolated, produced or synthesized such that it is separate from a complete, native HSV virus, although the isolated polypeptide may subsequently be introduced into a recombinant HSV or other virus. A recombinant virus that comprises an isolated polypeptide or polynucleotide of the invention is an example of subject matter provided by the invention. Preferably, such isolated polypeptides are at least about 90% pure, more preferably at least about 95% pure and most preferably at least about 99% pure. A polynucleotide is considered to be isolated if, for example, it is cloned into a vector that is not part of the natural environment.

The polypeptide can be isolated from its naturally occurring form, produced by recombinant means or synthesized chemically. Recombinant polypeptides encoded by DNA sequences described herein can be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably the host cells employed are E. coli, yeast or a mammalian cell line such as Cos or CHO. Supernatants from the soluble host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Fragments and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, wherein amino acids are sequentially added to a growing amino acid chain (Merrifield, 1963, J. Am. Chem. Soc. 85:2146-2149). Equipment for automated synthesis of polypeptides is commercially available from suppliers such as Perkin Elmer/Applied BioSystems Division (Foster City, Calif.), and may be operated according to the manufacturer's instructions.

Variants of the polypeptide for use in accordance with the invention can have one or more amino acid substitutions, deletions, additions and/or insertions in the amino acid sequence indicated that result in a polypeptide that retains the ability to elicit an immune response to HSV or HSV-infected cells. Such variants may generally be identified by modifying one of the polypeptide sequences described herein and evaluating the reactivity of the modified polypeptide using a known assay such as a T cell assay described herein. Polypeptide variants preferably exhibit at least about 70%, more preferably at least about 90%, and most preferably at least about 95% identity to the identified polypeptides over the length of the identified polypeptide. These amino acid substitutions include, but are not necessarily limited to, amino acid substitutions known in the art as “conservative”.

A “conservative” substitution is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

One can readily confirm the suitability of a particular variant by assaying the ability of the variant polypeptide to elicit an immune response. The ability of the variant to elicit an immune response can be compared to the response elicited by the parent polypeptide assayed under identical circumstances. One example of an immune response is a cellular immune response. The assaying can comprise performing an assay that measures T cell stimulation or activation. Examples of T cells include CD4 and CD8 T cells.

One example of a T cell stimulation assay is a cytotoxicity assay, such as that described in Koelle, D M et al., Human Immunol. 1997, 53; 195-205. In one example, the cytotoxicity assay comprises contacting a cell that presents the antigenic viral peptide in the context of the appropriate HLA molecule with a T cell, and detecting the ability of the T cell to kill the antigen presenting cell. Cell killing can be detected by measuring the release of radioactive ⁵¹Cr from the antigen presenting cell. Release of ⁵¹Cr into the medium from the antigen presenting cell is indicative of cell killing. An exemplary criterion for increased killing is a statistically significant increase in counts per minute (cpm) based on counting of ⁵¹Cr radiation in media collected from antigen presenting cells admixed with T cells as compared to control media collected from antigen presenting cells admixed with media.

### Fusion Proteins

The polypeptide can be a fusion protein. In one embodiment, the fusion protein is soluble. A soluble fusion protein of the invention can be suitable for injection into a subject and for eliciting an immune response. Within certain embodiments, a polypeptide can be a fusion protein that comprises multiple polypeptides as described herein, or that comprises at least one polypeptide as described herein and an unrelated sequence. In one example, the fusion protein comprises a HSV epitope described herein (with or without flanking adjacent native sequence) fused with non-native sequence. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., 1985, Gene 40:39-46; Murphy et al., 1986, Proc. Natl. Acad. Sci. USA 83:8258-8262; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located 5′ to the DNA sequence encoding the first polypeptides. Similarly, stop codons required to end translation and transcription termination signals are present 3′ to the DNA sequence encoding the second polypeptide.

Fusion proteins are also provided that comprise a polypeptide of the present invention together with an unrelated immunogenic protein. Preferably the immunogenic protein is capable of eliciting a recall response. Examples of such proteins include tetanus, tuberculosis and hepatitis proteins (see, for example, Stoute et al., 1997, New Engl. J. Med., 336:86-9).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenza virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In some embodiments, it may be desirable to couple a therapeutic agent and a polypeptide of the invention, or to couple more than one polypeptide of the invention. For example, more than one agent or polypeptide may be coupled directly to a first polypeptide of the invention, or linkers that provide multiple sites for attachment can be used. Alternatively, a carrier can be used. Some molecules are particularly suitable for intercellular trafficking and protein delivery, including, but not limited to, VP22 (Elliott and O'Hare, 1997, Cell 88:223-233; see also Kim et al., 1997, J. Immunol. 159:1666-1668; Rojas et al., 1998, Nature Biotechnology 16:370; Kato et al., 1998, FEBS Lett. 427(2):203-208; Vives et al., 1997, J. Biol. Chem. 272(25):16010-7; Nagahara et al., 1998, Nature Med. 4(12):1449-1452).

A carrier may bear the agents or polypeptides in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088).

### Polynucleotides, Vectors, Host Cells and Recombinant Viruses

The invention provides polynucleotides that encode one or more polypeptides of the invention. The polynucleotide can be included in a vector. The vector can further comprise an expression control sequence operably linked to the polynucleotide of the invention. In some embodiments, the vector includes one or more polynucleotides encoding other molecules of interest. In one embodiment, the polynucleotide of the invention and an additional polynucleotide can be linked so as to encode a fusion protein.

Within certain embodiments, polynucleotides may be formulated so to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, vaccinia or a pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

The invention also provides a host cell transformed with a vector of the invention. The transformed host cell can be used in a method of producing a polypeptide of the invention. The method comprises culturing the host cell and recovering the polypeptide so produced. The recovered polypeptide can be purified from culture supernatant.

Vectors of the invention can be used to genetically modify a cell, either in vivo, ex vivo or in vitro. Several ways of genetically modifying cells are known, including transduction or infection with a viral vector either directly or via a retroviral producer cell, calcium phosphate precipitation, fusion of the recipient cells with bacterial protoplasts containing the DNA, treatment of the recipient cells with liposomes or microspheres containing the DNA, DEAE dextran, receptor-mediated endocytosis, electroporation, micro-injection, and many other techniques known to those of skill. See, e.g., Sambrook et al. Molecular Cloning—A Laboratory Manual (2nd ed.) 1-3, 1989; and Current Protocols in Molecular Biology, F. M. Ausubel et al., eds., Greene Publishing Associates, Inc. and John Wiley & Sons, Inc., (1994 Supplement).

Examples of viral vectors include, but are not limited to retroviral vectors based on, e.g., HIV, SIV, and murine retroviruses, gibbon ape leukemia virus and other viruses such as adeno-associated viruses (AAVs) and adenoviruses. (Miller et al. 1990, Mol. Cell. Biol. 10:4239; J. Kolberg 1992, NIH Res. 4:43, and Cornetta et al. 1991, Hum. Gene Ther. 2:215). Widely used retroviral vectors include those based upon murine leukemia virus (MuLV), gibbon ape leukemia virus (GaLV), ecotropic retroviruses, simian immunodeficiency virus (SIV), human immunodeficiency virus (HIV), and combinations. See, e.g. Buchscher et al. 1992, J. Virol. 66(5):2731-2739; Johann et al. 1992, J. Virol. 66(5):1635-1640; Sommerfelt et al. 1990, Virol. 176:58-59; Wilson et al. 1989, J. Virol. 63:2374-2378; Miller et al. 1991, J. Virol. 65:2220-2224, and Rosenberg and Fauci 1993 in Fundamental Immunology, Third Edition, W. E. Paul (ed.) Raven Press, Ltd., New York and the references therein; Miller et al. 1990, Mol. Cell. Biol. 10:4239; R. Kolberg 1992, J. NIH Res. 4:43; and Cornetta et al. 1991, Hum. Gene Ther. 2:215.

In vitro amplification techniques suitable for amplifying sequences to be subcloned into an expression vector are known. Examples of such in vitro amplification methods, including the polymerase chain reaction (PCR), ligase chain reaction (LCR), Qβ-replicase amplification and other RNA polymerase mediated techniques (e.g., NASBA), are found in Sambrook et al. 1989, Molecular Cloning—A Laboratory Manual (2nd Ed) 1-3; and U.S. Pat. No. 4,683,202; PCR Protocols A Guide to Methods and Applications (Innis et al. eds.) Academic Press Inc. San Diego, Calif. 1990. Improved methods of cloning in vitro amplified nucleic acids are described in U.S. Pat. No. 5,426,039.

The invention additionally provides a recombinant microorganism genetically modified to express a polynucleotide of the invention. The recombinant microorganism can be useful as a vaccine, and can be prepared using techniques known in the art for the preparation of live attenuated vaccines. Examples of microorganisms for use as live vaccines include, but are not limited to, viruses and bacteria. In a preferred embodiment, the recombinant microorganism is a virus. Examples of suitable viruses include, but are not limited to, vaccinia virus and other poxviruses.

### Compositions

The invention provides compositions that are useful for treating and preventing HSV infection. The compositions can be used to inhibit viral replication and to kill virally-infected cells. In one embodiment, the composition is a pharmaceutical composition. The composition can comprise a therapeutically or prophylactically effective amount of a polypeptide, polynucleotide, recombinant virus, APC or immune cell of the invention. An effective amount is an amount sufficient to elicit or augment an immune response, e.g., by activating T cells. One measure of the activation of T cells is a cytotoxicity assay, as described in D. M. Koelle et al., 1997, Human Immunol. 53:195-205. In some embodiments, the composition is a vaccine.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The composition of the invention can further comprise one or more adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other viral antigens may be present, either incorporated into a fusion polypeptide or as a separate compound, within the composition or vaccine.

A pharmaceutical composition or vaccine may contain DNA encoding one or more of the polypeptides of the invention, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, 1998, Crit. Rev. Therap. Drug Carrier Systems 15:143-198, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., 1989, Proc. Natl. Acad. Sci. USA 86:317-321; Flexner et al., 1989, Ann. My Acad. Sci. 569:86-103; Flexner et al., 1990, Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91102805; Berkner, 1988, Biotechniques 6:616-627; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cir. Res. 73:1202-1207. Techniques for incorporating DNA into such expression systems are well known to those of ordinary skill in the art. The DNA may also be “naked,” as described, for example, in Ulmer et al., 1993, Science 259:1745-1749 and reviewed by Cohen, 1993, Science 259:1691-1692. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

Any of a variety of adjuvants may be employed in the vaccines of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes biodegradable microspheres; monophosphoryl lipid A and quil A. Cytokines, such as GM CSF or interleukin-2, −7, or −12, may also be used as adjuvants.

Within the vaccines provided herein, the adjuvant composition is preferably designed to induce an immune response predominantly of the Th1 type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, IL-5, IL-6, IL-10 and TNF-β) tend to favor the induction of humoral immune responses.

Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, 1989, Ann. Rev. Immunol. 7:145-173.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL™ adjuvants are available from Corixa Corporation (see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555. Another preferred adjuvant is a saponin, preferably QS21, which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprises an oil-in-water emulsion and tocopherol. A particularly potent adjuvant formulation involving QS21, 3D-MPL and tocopherol in an oil-in-water emulsion is described in WO 95/17210. Another adjuvant that may be used is AS-2 (Smith-Kline Beecham). Any vaccine provided herein may be prepared using well known methods that result in a combination of antigen, immune response enhancer and a suitable carrier or excipient.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets HSV-infected cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have antiviral effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic immunity (see Timmerman and Levy, Ann. Rev. Med. 50:507-529, 1999). In general, dendritic cells may be identified based on their typical shape (stellate in situ, with marked cytoplasmic processes (dendrites) visible in vitro) and based on the lack of differentiation markers of B cells (CD19 and CD20), T cells (CD3), monocytes (CD14) and natural killer cells (CD56), as determined using standard assays. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (Zitvogel et al., 1998, Nature Med. 4:594-600).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce maturation and proliferation of dendritic cells.

Dendritic cells are conveniently categorized as “immature” and “mature” cells, which allows a simple way to discriminate between two well-characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor, mannose receptor and DEC-205 marker. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80 and CD86).

APCs may generally be transfected with a polynucleotide encoding a polypeptide (or portion or other variant thereof) such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75:456-460. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

### Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Preferably, the patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue.

The compositions are administered in any suitable manner, often with pharmaceutically acceptable carriers. Suitable methods of administering cells in the context of the present invention to a patient are available, and, although more than one route can be used to administer a particular cell composition, a particular route can often provide a more immediate and more effective reaction than another route.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases such as HSV infection, the physician needs to evaluate the production of an immune response against the virus, progression of the disease, and any treatment-related toxicity.

For example, a vaccine or other composition containing a subunit HSV protein can include 1-10,000 micrograms of HSV protein per dose. In a preferred embodiment, 10-1000 micrograms of HSV protein is included in each dose in a more preferred embodiment 10-100 micrograms of HSV protein dose. Preferably, a dosage is selected such that a single dose will suffice or, alternatively, several doses are administered over the course of several months. For compositions containing HSV polynucleotides or peptides, similar quantities are administered per dose.

In one embodiment, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an antiviral immune response, and is at least 10-50% above the basal (i.e., untreated) level. Such vaccines should also be capable of causing an immune response that leads to an improved clinical outcome in vaccinated patients as compared to non-vaccinated patients. In general, for pharmaceutical compositions and vaccines comprising one or more polypeptides, the amount of each polypeptide present in a dose ranges from about 0.1 μg to about 5 mg per kg of host. Preferably, the amount ranges from about 10 to about 1000 μg per dose. Suitable volumes for administration will vary with the size, age and immune status of the patient, but will typically range from about 0.1 mL to about 5 mL, with volumes less than about 1 mL being most common.

Compositions comprising immune cells are preferably prepared from immune cells obtained from the subject to whom the composition will be administered. Alternatively, the immune cells can be prepared from an HLA-compatible donor. The immune cells are obtained from the subject or donor using conventional techniques known in the art, exposed to APCs modified to present an epitope of the invention, expanded ex vivo, and administered to the subject. Protocols for ex vivo therapy are described in Rosenberg et al., 1990, New England J. Med. 9:570-578. In addition, compositions can comprise APCs modified to present an epitope of the invention.

Immune cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to enrich and rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., 1997, Immunological Reviews 157:177).

Administration by many of the routes of administration described herein or otherwise known in the art may be accomplished simply by direct administration using a needle, catheter or related device, at a single time point or at multiple time points.

### Methods of Treatment and Prevention

The invention provides a method for treatment and/or prevention of HSV infection in a subject. The method comprises administering to the subject a composition of the invention. The composition can be used as a therapeutic or prophylactic vaccine. In one embodiment, the HSV is HSV-1. Alternatively, the HSV is HSV-2. The invention additionally provides a method for inhibiting viral replication, for killing virally-infected cells, for increasing secretion of lymphokines having antiviral and/or immunomodulatory activity, and for enhancing production of virus-specific antibodies. The method comprises contacting an infected cell with an immune cell directed against an antigen of the invention, for example, as described in the Examples presented herein. The contacting can be performed in vitro or in vivo. In a preferred embodiment, the immune cell is a T cell. T cells include CD4 and CD8 T cells. Compositions of the invention can also be used as a tolerizing agent against immunopathologic disease.

In addition, the invention provides a method of producing immune cells directed against HSV. The method comprises contacting an immune cell with a polypeptide of the invention. The immune cell can be contacted with the polypeptide via an antigen-presenting cell, wherein the antigen-presenting cell is modified to present an antigen included in a polypeptide of the invention. Preferably, the antigen-presenting cell is a dendritic cell. The cell can be modified by, for example, peptide loading or genetic modification with a nucleic acid sequence encoding the polypeptide. In one embodiment, the immune cell is a T cell. T cells include CD4 and CD8 T cells. Also provided are immune cells produced by the method. The immune cells can be used to inhibit viral replication, to kill virally-infected cells, in vitro or in vivo, to increase secretion of lymphokines having antiviral and/or immunomodulatory activity, to enhance production of virus-specific antibodies, or in the treatment or prevention of viral infection in a subject.

### EXAMPLES

The following examples are presented to illustrate the present invention and to assist one of ordinary skill in making and using the same. The examples are not intended in any way to otherwise limit the scope of the invention.

### Example 1

### Herpes Simplex Virus Type 1 T-Cell Antigens in Humans Revealed by Cross-Presentation and Genome-Wide Screening

There is an unmet need for vaccines for herpes simplex virus type 1 (HSV-1) and other large-genome pathogens. Presumably, a vaccine must stimulate coordinated T-cell responses, but the large genome and the low frequency of virus-specific T-cells have hampered the search for T-cell antigens. We have developed new methods to efficiently provide a genome-wide map of HSV-1-specific T-cells, and demonstrate applicability to a second complex microbe, vaccinia virus. We used cross-presentation and CD137 activation-based FACS to enrich polyclonal CD8 effectors. The HSV-1 proteome was prepared in a flexible format for CD8 and CD4 studies. Scans with participant-specific artificial APC panels identified an oligospecific response in each person. Results enabled streamlined discovery of myriad novel CD8 epitopes, permitting direct ex vivo assays of HLA-appropriate persons. Parallel CD137-based CD4 research showed discrete oligospecific recognition of HSV-1 antigens. Unexpectedly, the two HSV-1 proteins not previously considered as vaccine candidates elicited both CD8 and CD4 T-cells in most HSV-1-infected persons. These new methods, also demonstrated in principle for vaccinia virus for both CD8 and CD4 T-cells, should be broadly applicable to T-cell vaccine antigen selection in the era of microbial genomics.

HSV immune evasion and the low abundance of HSV specific CD8 cells in human blood impair their study. Inhibition of TAP, down-regulation of HLA class I (28, 29), decreased DC co-stimulation (30), and disruption of TCR signaling (31, 32) mediated by various HSV genes all likely contribute to difficulties with direct presentation in in vitro settings. In contrast, murine HSV data show that both the priming of naïve CD8 responses and the recall of memory CD8 cells use cross—rather than direct priming and presentation (33-38).

We earlier showed that human monocyte-derived DC (moDC) can cross-present HSV-2 to memory HSV-2-specific CD8s (39). In this report, we harnessed cross-presentation to stimulate HSV-1-specific CD8 T-cells, while CD4 memory cells were reactivated with whole killed antigen. CD137 was then used to radically enrich HSV-1-specific T-cells. CD137 is a tumor necrosis factor receptor family protein that identifies recently activated CD4 and CD8 T-cells (40-42). A near complete collection of HSV-1 molecular clones was deployed in specific CD8 and CD4 formats to perform genome-wide scans of these polyclonal virus-specific T-cells. Our data identified the proteins expressed by HSV-1 genes UL39 and UL46, previously not known to be CD8 T-cell antigens, as rational vaccines candidates for the elicitation of coordinated CD8 and CD4 responses. In contrast, gD1, the homolog of gD2, was a poor CD8 antigen.

To test the broad applicability of our methods, we evaluated vaccinia virus, a microbe with over 200 genes. We studied if moDC cross-presentation and CD137-based methods using whole virus would enrich vaccinia-reactive CD8 and CD4 T-cells. The affirmative results suggest that a modular approach to T-cell antigen discovery and prioritization, modified on a case-by-case basis to reflect the biology of specific microbes, should be laterally transferable to challenging pathogens with large genomes.

### Methods

Participants and Specimens.

Healthy adults with HSV-1 infection were recruited at the University of Washington Virology Research Clinic. Participants were antibody negative for HIV-1. HSV-1 and HSV-2 infections were documented with type-specific serum immunoblot (70). Vaccinia immune status was derived from history of vaccinia vaccination. None of the participants had an oral or genital recurrence or received anti-herpesviral medication at the time of lymphocyte collection. The collection of PBMC from vaccinia-immune persons has been documented (71). Peripheral venous blood was collected with heparin and processed by Ficoll density gradient centrifugation (72). A 150 ml blood sample allowed CD8 and CD4 analyses for HSV-1. PBMC were cryopreserved until use.

HLA Typing.

HLA at A and B loci was typed at the Puget Sound Blood Center, Seattle, Wash. When 2 digits followed by xx or 4 digits are reported, low or high resolution typing was done and the old nomenclature used (73). HLA C was typed by Dr. Dan Geraghty at the Fred Hutchinson Cancer Research Center by sequencing exons 2 and 3. Ambiguous HLA C alleles are reported to 4 digits (predicted amino acid sequence) using the older nomenclature if there are only two possibilities. Some HLA C alleles are reported using the new G nomenclature (http://hla.alleles.orq/alleles/g_qroups.html) when there are multiple possible amino acid sequences (73).

Virus and Cell Culture.

HSV-1 strain E115 (74) and HSV-2 strain 333 (75) were grown and titered (62) on Vero cells (ATCC). Vaccinia strain WR was grown and titered as described (76). Viral antigens for CD4 cell stimulation and readout assays were diluted sonicates of Vero (HSV-1) or BSC40 (vaccinia) harvested by scraping at 4+ cytopathic effect and treated with UV light to eliminate infectious virus (72, 77). HeLa and Cos-7 cells (ATCC) were cultured in DMEM. B-LCL were immortalized from PBMC using EBV strain B95-8 (62) and are permissive for HSV infection (78). Continuous cell lines were Mycoplasma negative (Lonza). Monocyte-derived DC (moDC) were cultured from adherent or CD14 positively-selected (Miltenyi) PBMC using media, conditions, and recombinant human granulocyte monocyte colony stimulating factor (GM-CSF) and IL-4 as described (53). Media (72, 79) used 10% Fetalclone III (Fisher) instead of FCS.

Enrichment and Expansion of Virus-Reactive T-Cells from PBMC.

For HSV-1-specific CD8 T-cell enrichment, HeLa cells, seeded 1-2 days before in 6-well plates at 3×10⁵ cells/well and used at 80-90% confluence, were infected with HSV-1 at a MOI of 5, or a similar dilution of mock virus, for 30 min with rocking in serum-free medium at 37° C., 5% CO₂, followed by addition of complete medium. At 18 h, cytopathic effect was visible by microscopy. Cells were recovered by 20 mM EDTA and scraping, washed in TCM, re-suspended at 2×10⁶/ml in TCM, plated in a 6-well plate, and exposed to UV radiation (Stratalinker XL1000, Agilent, 180,000 microjoules). MoDC were similarly recovered from their culture wells, washed, and re-suspended at 2×10⁶/ml in TCM and plated at 100 μl/well in 48-well plates. HeLa debris (100 μl/well, equivalent to 2×10⁵ cells/well prior to UV) was added for a moDC/HeLa ratio of 1:1 for 5 h at 37° C., 5% CO₂. CD8+ T-cells were negatively selected from autologous PBMC (Miltenyi) and added (1×10⁶/well in 300 μl) to the antigen-loaded moDC for a responder:DC ratio of 10:1 in a final volume of 500 μl TCM/well. Antigen loading and stimulations typically involved 15-20 identical wells/person. Vaccinia-specific CD8 T-cells were re-stimulated by cross-presentation as described for T-cell clones (80) except that bulk PBMC-derived CD8+ T-cells were used as responders. The remainder of the procedure matched that for HSV-1.

Stimulation was for 20 h at 37° C., 5% CO₂. Cells were pooled and stained with 7-AAD, anti-CD3-PE, anti-CD8α-FITC, and anti-CD137-allophycocyanin (Becton Dickinson) for 30 min at room temperature in 50 μl TCM. After 2 washes, cells were re-suspended in 1×10⁷/ml at TCM. FACSAria II (Becton Dickenson) FACS used initial gating of live CD3+ lymphocytes. All available CD8+ CD137^{high} cells were collected as were a fraction of the abundant CD137-negative cells. Sorted cells were washed and plated in bulk with 1.5×10⁵ allogeneic 3300-rad irradiated PBMC and 1.6 μg/ml PHA-P (Remel) in 200 μl TCM in a 96-well U-bottom. Natural hIL-2 (32 U/ml, Hemagen) was added on day 2 and maintained for 14-16 days, typically yielding 1−10×10⁶ cells. A portion of the output cells from the first expansion were bulk stimulated with anti-CD3 mAb, feeder cells, and recombinant hIL-2 (79). This typically yielded a 1000-fold cell increase in 14 days (81). Frozen sister aliquots of expanded cells were thawed for downstream assays. Re-expansion was not required.

Enrichment of virus-specific CD4 T-cells began with adding UV-inactivated, cell-associated HSV-1 or vaccinia (MOI prior to inactivation) to 2×10⁶ PBMC per well of 24 well plates in 2 ml TCM. This HSV preparation has been proven to contain non-structural proteins such as the UL50-encoded enzyme, and structural proteins (82). Cultures were initiated from 15−20×10⁶ PBMC. After 20 h, cells with stained as above but anti-CD4 was substituted for anti-CD8α. Live CD3+CD4+CD137+ cells, and a portion of the CD3+CD4+CD137− cells were sorted and expanded in bulk as above.

HSV-1 ORFeome.

Total DNA was prepared from cells infected with HSV-1 strain 17+(14) using the Qiagen blood kit cultured cell instructions. PCR primers were designed to amplify each annotated open reading frame (ORF) in HSV-1 (Genbank NC_{—}001806). Whenever possible, full-length ORFs were amplified. For ICP0, each exon was amplified independently. Since UL 15 has an intron, its' entry vector was generated using a cDNA clone as PCR template. Nomenclature was from a standard reference (1). In some cases, long ORFs were amplified as fragments, usually overlapping by several amino acids. These were labeled as fragment (frag) A, etc. in the N- to C-terminal direction. In other cases, both a full-length clone and an internal fragment or fragments were separately cloned; the first internal fragment is labeled as frag A if it starts near the N-terminus and frag B if not. ORFs that are internal to, in-frame with, and therefore have polypeptide sequences identical to fragments of longer open reading frames, such as ORF UL26.5 within UL26 (1), were separately cloned in some instances but are not separately reported (see below). For each gene or fragment, both primers had a 5′ extension to allow recombinase-mediated integration into pDONR207 or pDONR221 (Invitrogen), yielding vectors termed pENTR207-gene X or pENTR221-gene X. Recombination used Invitrogen reagents. Plasmids recovered from candidate bacterial colonies were evaluated by restriction endonucleases and/or sequencing.

Vector pDEST103, for CD8 work, accepts DNA inserts from either pDONR207 or pDONR221, and express polypeptides of interest such that EGFP is at the N terminus of a fusion polypeptide. To create pDEST103, peGFP-C1 (Clontech) was linearized with Xho I, blunt-ended with T4 DNA polymerase and dNTPS, dephosphorylated with calf intestinal alkaline phosphatase, and gel-purified. Reading frame cassette A (Invitrogen) was ligated with T4 DNA ligase into peGFP-C1. After selection with chloramphenicol and kanamycin in E. coli strain ccdB survival 2 T1^{R}, intermediary vector pDEST102 was recovered and the orientation of the cassette was confirmed by sequencing. pcDNA3.1(+) (Invitrogen) was digested with Nhe I and Hind III. pDEST102 was similarly digested and the insert, comprised of EGFP and Cassette A with termini was gel purified and ligated into digested pcDNA3.1(+), creating pDEST103. pDEST103 expressed the ccdB-encoded protein and was with selected with ampicillin and chloramphenicol in ccdB survival E. coli. The identity of pDEST103 was confirmed by sequencing through att recombination sites, EGFP, and flanking regulatory regions. HSV-1 inserts were transferred from either of the pENTR series vectors (above) to pDEST103 with LR Clonase II (Invitrogen) and selected using ampicillin in E. coli DH5α, yielding pEXP103-gene X vectors. Candidate pEXP103-gene X expressing HSV-1 polypeptides were sequencing through their EGFP-HSV junctions at the N-termini of the HSV polypeptide, and through the C-termini insert-vector junctions. The fusion polypeptides are predicted to encode EGFP, followed by peptide SGLRCRITSLYKKAGF (seqid), followed by the HSV-1 polypeptide of interest. DNA was prepared using anion exchange (Qiagen), measured at OD₂₆₀ (Nanodrop) and diluted in water (100 ng/μl) for transfection.

Expression of each HSV-1 ORF was checked by transfecting Cos-7 cells cultured in 96-well flat bottom plates as described (81) with 100 ng/well DNA. 48 h later, cells recovered by trypsinization, stained with Violet live/dead (Invitrogen), and analyzed for EGFP by flow cytometry after gating on live cells. For protein gD1, pEXP103-US6 expression was confirmed with a mAb and flow cytometry as described (83). For protein gB1, pEXP103-UL27 expression was confirmed using the same technology except that mAb H1817 (Novus) was used at 5 μl/tube as the primary antibody and PE-conjugated goat anti-mouse IgG (Invitrogen) was used at 1 μl/tube as secondary.

Each predicted HSV-1 polypeptide from the pENTR series (above) was separately subcloned into custom vector pDEST203 designed for in vitro protein expression and CD4 research. To construct pDEST203, pIVEX2.4d (Roche) was digested with Xho I, blunt-ended with T4 DNA polymerase and dNTPs, de-phosphorylated, and ligated with the reading frame B cassette (Invitrogen). Colonies were selected in ccdB survival E. coli as above but with ampicillin and chloramphenicol. A sequence-confirmed correct plasmid was termed pDEST203. pDEST203 has a T7 promoter, a transcriptional unit encoding 6-Histidine fused to the HSV-1 polypeptide, attR recombination sites, and features suitable for in vitro transcription/translation. HSV-1 inserts from pENTR207 or pENTR221 were moved to pDEST203 using LR Clonase II to generate the pEXP203-ORF series. The left and right vector-insert junctions of each pEXP203-ORF plasmid were sequenced to confirm identity and in-frame fusion with 6-His. Each pEXP203 construct encodes MSGSHHHHHHSSGIEGRGRLIKHMTMASRLESTSLYKKAGF (SEQ ID NO: 68) at the N-terminal, followed in-frame by the HSV-1 polypeptide.

For expression, pEXP203 plasmids were prepared from a 3 ml ampicillin culture of transformed E. coli using a silica method (Qiagen) and mass determined by spectrophotometry. Protein expression used 50 μl volumes of the Expressionway E. coli system (Invitrogen). To check expression, 1 μl of reaction product was spotted onto nitrocellulose membranes (Whatman, Piscataway, N.J.), air-dried, blocked with 1% blocking reagent (Roche) diluted in TBS (50 mM Tris, 150 mM NaCl, pH 7.5), probed with anti-6-His mAb (Roche) diluted 1:500 in TBS-0.1% Tween −20 (TBS-T) (Roche), washed with TBS-T, incubated with horseradish peroxidase-conjugated anti-mouse IgG (Promega) diluted 1:2500 in TBS-T, washed with TBS-T, and developed with TMB substrate (Promega). Proteins failing to display a spot indicating anti-6-His binding were re-synthesized. For HSV-1 protein VP22 (gene UL49), expression was checked by triplicate, 3-day ³H thymidine proliferation assay using cornea-derived CD4+ clone 9447.28 specific for HSV-1 VP22 AA 199-211 (18) as responders (2×10⁴/well), autologous 3,300 rad γ-irradiated PBMC as APC (10⁵/well), and HSV-1 VP22 or controls expressed in the pEXP203 system.

HLA cDNA Cloning.

HLA class I cDNAs for A*0101, A*0201, A*2902, B*0702, B*0801, B*4402, and B*5801 are documented (52, 56, 71, 84). Cloning of HLA A*2402, A*2601, and A*6801 used 5′ primer CCGCCGCTAGCATGGCCGTCATGGCGCCCCGA (SEQ ID NO: 69) and 3′ primer CCGCCCTCGAGTCACACTTTACAAGCTGTG (SEQ ID NO: 70). Cloning of HLA B*1516, B*3503, B*5101, and B*5801 used 5′ primer CCGCCGCTAGCATGCGGGTCACGGCGCCCCGAACCG (SEQ ID NO: 71) and 3′ primer CCGCCCTCGAGTCAAGCTGTGAGAGACACATCAG (SEQ ID NO: 72). Cloning of HLA Cw0704 used 5′ primer CCGCCTGCTAGCATGCGGGTCATGGCGCCCCGAG (SEQ ID NO: 73) and 3′ primer CCGCCCGTCTCGAGTCAGGCTTTACAAGTGATGAGAGAC (SEQ ID NO: 74). Cloning of HLA Cw0102, Cw0202, Cw0401, Cw0501, Cw0701, Cw0702, and Cw1402 used 5′ primer TATAAAGCTTTTCTCCCCAGACGCCGAGA (SEQ ID NO: 75) and 3′ primer ATATGCGGCCGCGTCTCAGGCTTTACAAGCGA (66; SEQ ID NO: 76). For new alleles, RNA was prepared from PBMC or B-LCL (79) (RNeasy kit, Invitrogen). cDNA synthesis used random hexamer primers (HLA A, B, and Cw0704) or oligo-dT (other HLA C alleles) and Superscript II (Invitrogen). PCR used the above primer pairs (bold Nhe I and Xho I sites for HLA A, B, and Cw0704; Hind III and Not I for other HLA C; additional distal non-HLA sequences in italics; HLA-specific sequences in plain font with ATG start codons underlined for HLA A, B, and Cw0704). PCR amplicons at the expected MW were digested with the restriction enzymes listed and cloned into pcDNA3.1(+) (Invitrogen) (HLA, B, and Cw0704) or pcDNA3.1/V5-His TOPO (Invitrogen) (other HLA C). cDNA clones with 100% sequence matches (85) were prepared by anion exchange (Qiagen).

To validate expression, Cos-7 cells were transfected with plasmid cDNA and Fugene 6 (Boehringer Mannheim-Roche), for 48 h, trypsinized, and surface stained for flow cytometry with anti-HLA mAb (A*0101: 0544HA; A*0201: MA2.1 (86); A*2402: 0497HA; A*2601: 0544HA; A*2902: 0334HA; B*0801: 0059HA; B*1516: 0044HA; B*3503: 0789AHA; B*4402: 0786BHA; One Lambda, except MA2.1 as described (56)). Isotype-appropriate fluorochrome-labeled polyclonal antibodies were used to detect primary antibody binding (81). Negative controls were Cos-7 cells stained with secondary antibody only, non-transfected Cos-7 with both antibodies, and HLA-mismatched B-LCL; positive controls were HLA-matched B-LCL. In some situations, HLA alleles were matched to participants based on low resolution typing and knowledge of the most prevalent HLA allelic subtypes. Specifically, for participant 2 with HLA B*07 and B*08 were used the most likely alleles, B*0702 and B*0801; for participant 5 with HLA A*01 and B*08 and B*51 we used A*0101, B*0801 and B*5101; and for participant 6 with A*24 we used A*2402. For one participant at each locus, only one genome scan was done for CD8 responses due to homozygosity or near homozygosity. Specifically, participant 7 had HLA A*0220 and A*0224 (each differing from A*0201 at a single amino acid) for whom we used A*0201 only. Participant 6 had both B*3503 and B*3502 (differing by 3 amino acids from B*3503) for which only B*3503 was studied, and was homozygous for HLA C*04G1 for which Cw0401 was studied. Participant 4 had A*2901, but we used A*2902 differing at one amino acid.

ORFeome CD8 Screens.

Cos-7 cells were plated in 96-well flat plates as described (56) and 24 h later were simultaneously transfected with 50 ng HLA cDNA and 150 ng/well pDEST103-based HSV-1 construct. Each HSV-1 ORF or fragment was assayed in duplicate. Negative controls were pDEST103 mono-transfected. After 48 h, bulk polyclonal CD8 effector cells (above) were added at 5−10×10⁴/well in 200 μl fresh TCM. After 16-24 h, supernatants were collected and stored at −20° C. T-cell activation was detected by supernatant ELISA for IFN-γ (56).

ELISA data were designed to classify each HSV-1 ORF/HLA transfection combination as positive or negative for each responder T-cell line. For an ORF to be considered positive, we required that both individual OD₄₅₀ readings were 0.08 or greater for every screen except for one with higher background, where the threshold was set at 0.1. For ORFs screened as more than one fragment, or both a full length and one or more internal fragments or separately annotated but in-frame genes, or exon by exon, the major ORF was considered the fundamental unit of analysis and was considered positive if one or more fragment(s) scored positive. Analyses grouped proteins by the kinetics of gene expression in the context of infected cells or by structural or functional biology from reviews (1) and primary literature, as well as by presence or absence from virions (51).

ORFeome CD4 Screens.

Bulk-expanded HSV-1-reactive CD4 T-cell lines were tested for proliferative responses to individual HSV-1 proteins as described for vaccinia (87, 88). Briefly, 5−10×10⁴ autologous gamma-irradiated (3300 rad) PBMC, 3×10⁴ bulk responder cells, and recombinant HSV-1 proteins (above) diluted 1:5000 were plated in duplicate in 200 μl TCM in 96-well U-bottom plates. Negative controls included similar dilutions of the in vitro transcription/translation products of plasmids encoding Francisella tularensis proteins, empty pDEST203, or no DNA. F. tularensis ShuS4 molecular clones of genes 1208, 1127, 1305, 7056, 1306, 1314, 1961, 5396, 1852, 1254, 1729, 1695, 1544, 1835, and 1963 in vector pXT7 (89) were maintained in E. coli under kanamycin selection. Protein was synthesized as described for HSV-1. UV-treated HSV-1 and mock virus were positive and negative controls. ³H thymidine incorporation was measured after 3 days (47). The criteria for designating an HSV-1 ORF or fragment as positive used a the negative controls (n=30) and was set as the mean plus 3.09 times the standard deviation of the negative controls (n=30) (47) for a one-tailed false discovery rate of 0.1%

Intracellular Cytokine Cytometry.

The reactivity of T-cell responder cultures was tested by intracellular cytokine cytometry (ICC) as described (87). The format for checking CD8 reactivity with whole virus involved infected autologous B-LCL with mock virus, HSV-1, HSV-2, or vaccinia for 18 h at MOI 5, washing, and co-culturing 2×10⁵ B-LCL with 2×10⁵ responder T-cells in 1 ml TCM. For CD4 reactivity, 2×10⁵ autologous, CFSE-labeled PBMC were co-cultured with 2×10⁵ responder T-cells and UV-treated HSV-1, HSV-2, vaccinia, or mock virus at 1:100 dilution in 1 ml TCM. For peptides, responder T-cells were co-cultured with equal numbers (˜10⁵) of CFSE pre-labeled autologous B-LCL and 1 μg/ml peptide or an equivalent volume of DMSO as negative control. Either staphylococcal enterotoxin B (SEB, 1 μg/ml, Becton Dickinson) or PMA and ionomycin (88) were used as positive control. Anti-CD28 and anti-CD49d were added at assay set-up, and Brefeldin A was used to reduce cytokine secretion. Cells were surface-stained for CD8 or CD4 as appropriate, permeabilized, and stained intracellularly for IFN-γ, and in some cases also for TNF-α and IL-2 (80). After appropriate washes and fixation, CFSE-negative cells in the lymphocyte forward and side scatter gates were analyzed by flow cytometry for binding of fluorochrome-labeled antibodies. For peptides, responses were considered positive if a discrete group of IFN-γ-expressing cells was observed and the percentage of IFN-γ+ cells was >2×DMSO background. Low-positive peptide responses were repeated at least once including new negative controls and only repeatedly reactive peptide responses were considered positive and were reported herein.

Cytolysis Assays.

The cytolytic activity of sorted, bulk, polyclonally expanded T-cell responder cultures was tested in ⁵¹Cr release assays (90). Briefly, target cells were created by infecting autologous or HLA class I mismatched allogeneic B-LCL with HSV-1 or vaccinia at MOI 5 for 18 h (90) while labeling with ⁵¹Cr. Washed target cells (2×10³/well) were co-cultured in triplicate in 96-well U-bottom plates with a 40-fold excess of responder cells for 4 h at 37° C., 5% CO₂ Media or 5% Igepal (Sigma) were used for spontaneous and total ⁵¹Cr release, respectively. 30 μl of supernatant was counted using Lumaplates and a TopCount (Packard). Data are reported as percent specific release (90); spontaneous release (90) was typically less than 20%.

ELISPOT.

To test PBMC directly ex vivo, duplicate IFN-γ ELISPOT was done as described except that un-manipulated PBMC were used (53). Thawed PBMC were tested at 7.5×10⁶/well. Stimuli were HSV-1 peptides (1 μg/ml, n=41), 0.1% DMSO negative control, and UV-killed HSV-1 at 1:100 dilution as positive control. Five peptides were omitted: HSV-1 UL53 201-209 (HLA A*0101 restricted), UL26 326-334 and UL27 641-649 (HLA A*2909 restricted), and US7 22-30 and ICP0 698-706 (HLA B*0702 restricted). Potential positives were manually reviewed using high density images. Peptides with >10 spot forming units (SFU) per 10⁶ PBMC and >2×DMSO background were considered positive.

HSV-1 Peptides.

The predicted amino acid sequences for HSV-1 ORFs or fragments that were reactive in CD8 ORFeome analyses were submitted with the restricting HLA class I alleles to binding prediction algorithms (91). Top-ranking 9-mers or 10-mers were synthesized with native termini, usually 3 to 10 per ORF per HLA allele (Sigma). We also purchased peptides gD1 (gene US6) 77-85 (SLPITVYYA), 94-102 (VLLNAPSEA), and 302-310 (ALLEDPVGT) reported to bind HLA A*0201 tightly and to be A*0201-restricted epitopes in gD1 (16). Peptide HSV-1 UL25 367-375 was made as described (54). Throughout, amino acid 1 refers to the genomic ATG encoding methionine, rather than alternative numbering schemes (16). The manufacturer characterized peptide MW by mass spectroscopy. Peptides were diluted to 10 mg/ml in DMSO, stored at −20° C. and further diluted in TCM.

Statistical Analysis.

For CD4 ORFeome screens, HSV-1 ORFs with mean CPM values above the mean plus 3.09 times the standard deviation for negative controls were considered positive for a one-tail false discovery rate of 0.1% (87). This q statistic for false discovery rates is generally considered to be analogous to the P value; in this case 0.1% corresponds to P of 0.001. For CD8 ORFeome screens, the mean background raw IFN-γ ELISA OD₄₅₀ values were typically 0.05 and were normally distributed. ORFs for which each duplicate well yielded an IFN-γ ELISA OD₄₅₀ value >2× the mean background value raw OD₄₅₀ (typically 0.08) were considered positive.

Study Approval.

Healthy adults with HSV-1 infection or a history of vaccinia vaccination were recruited into a protocol approved by the University of Washington IRB, Seattle, Wash., USA. Participants provided informed written consent.

### Results

HSV-1-Specific CD8 T-Cells can be Detected and Enriched by Cross-Presentation.

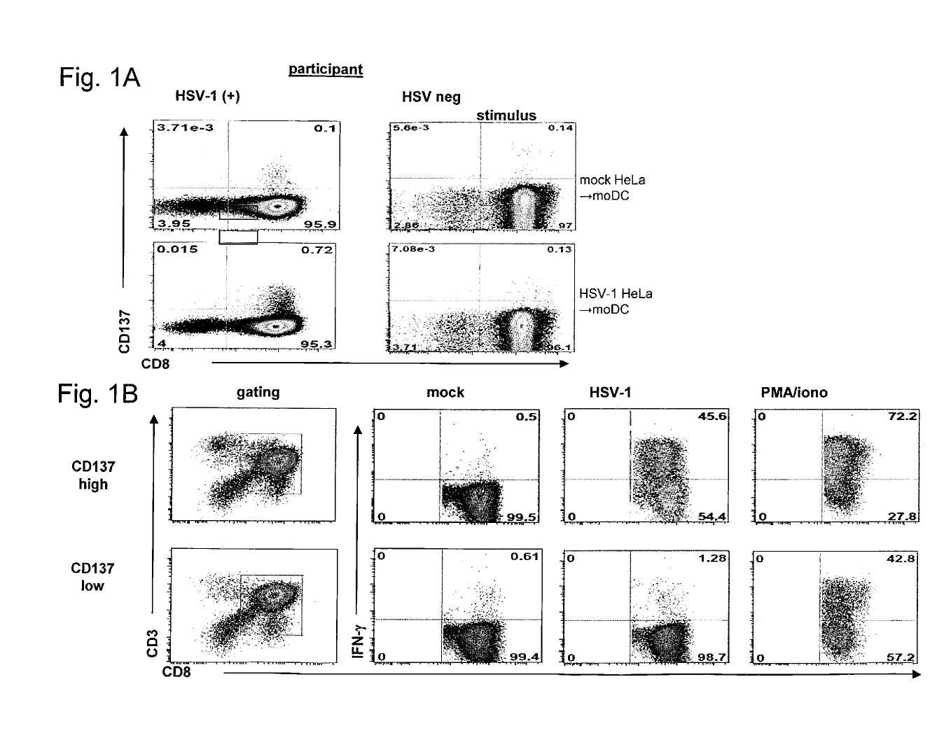

We studied seven HSV-1 seropositive persons (Table 1) in whom we measured HSV-1-specific T-cell responses in fine detail. In each case, exposure of CD8 cells from PBMC to autologous, HSV-1 antigen-loaded moDC for 20 h resulted in detectable CD137 responses amongst live, CD3+ CD8+ cells (representative participant, FIG. 1A). We observed that specific expression of CD137 was usually somewhat higher than for IFN-γ (Table 2). Amongst three individuals seronegative for HSV-1 and HSV-2 (persons 12-14, Table 1), two had very low CD137 and cytokine CD8 responses, while participant 13 had detectable responses in both assays (Table 2).

TABLE 1
Participants studied in this report.

| | | | HSV- | HSV- | | | | HLA | HLA | HLA |
|---|---|---|---|---|---|---|---|---|---|---|
| ID | age^{a} | gender | 1^{a} | 2^{a} | HLA^{b} A | HLA B | HLA C | DRB1 | DRB3,4,5 | DQB1 |
| 1 | 29 | M | pos | neg | *01xx, | *5101, * | *0202, | *0701 | | *0202 |
| | | | | | *0201 | 5801 | *07:01:01G | | | |
| 2 | 42 | F | pos | pos | *01xx, | *07xx, | *0702/0750, | *1501, | 3*0301, | *0602, |
| | | | | | *26xx | *08xx | *07:01:01G | *0301 | 5*0101 | *0201 |
| 3 | 24 | F | pos | pos | *2902, | *1516, * | *0704/0711, | *0401, | 3 pos, 4 | *0301 |
| | | | | | *6801 | 4402 | *1402 | *1101 | pos | |
| 4 | 42 | F | pos | pos | *2901, | *3503, * | *04:01:01G1, | *0401, | 3 pos, 4 | *0301, |
| | | | | | *6801 | 4402 | *0704/0711 | *1104 | pos | *0302 |
| 5 | 44 | M | pos | pos | *01xx, | *08xx, * | *0102, | *0101, | 3 pos | *0201, |
| | | | | | *0201 | 51xx | *07:01:01G | *0301 | | *0501 |
| 6 | 36 | M | pos | pos | *0201, | *3502, | *04:01:01G | *1104, | 3 pos | *0301, |
| | | | | | *24xx | *3503/3570 | | *1401 | | *0503 |
| 7 | 40 | M | pos | pos | *0220, | *4402, | *0501/0503, | *0101, | 4 pos | *0201, |
| | | | | | *0224 | *0702 | *0702/0750 | *0701 | | *0501 |
| 8 | 29 | F | pos | neg | not done | | | | | |
| 9 | 29 | F | pos | neg | not done | | | | | |
| 10 | 20 | F | pos | neg | not done | | | | | |
| 11 | 20 | F | pos | neg | not done | | | | | |
| 12 | 55 | M | neg | neg | *02xx | *51xx, | *01xx, *03xx | *0101, | 5 pos | *0501, |
| | | | | | | *60xx | | *1501 | | *0602 |
| 13 | 45 | F | neg | neg | not done | | | | | |
| 14 | 50 | M | neg | neg | *02xx, *6601/ | *38xx, | *12xx | *16xx, | 3 pos, 5 | *03xx, |
| | | | | | 6604 | *39xx | | *11xx | pos | *05xx |

^{a}At time of phlebotomy.
^{b}HLA nomenclature at A and B per older system (92); HLA C uses newer system (73). See Materials and Methods. The presence or absence of a functional allele at the HLA DRB3, 4, and 5 loci is indicated for some persons and the alleles identified for others.

TABLE 2
Direct ex vivo expression of intracellular IFN-γ or surface
CD137 by live, CD3+CD8+ cells after cross-presentation
by autologous moDC loaded with HSV-1 or control antigen.

| | IFN-γ | | | CD137 | | |
|---|---|---|---|---|---|---|
| person | mock | HSV-1 | net | mock | HSV-1 | net |
| | HSV seropositive | | | | | |
| 1 | 0.08 | 0.59 | 0.51 | 0.54 | 1.68 | 1.14 |
| 2 | not done | not done | not done | 0.12 | 2.63 | 2.51 |
| 3 | 0.07 | 0.11 | 0.04 | 0.22 | 1.93 | 1.71 |
| 4 | 0.01 | 0.10 | 0.09 | 0.12 | 0.52 | 0.40 |
| 5 | 0.01 | 0.19 | 0.18 | 0.51 | 0.78 | 0.27 |
| 6 | 0.16 | 2.54 | 2.38 | 0.21 | 2.96 | 2.75 |
| 7 | 0.07 | 0.19 | 0.12 | 0.23 | 1.52 | 1.29 |
| | HSV seronegative | | | | | |
| 12 | 0.07 | 0.08 | 0.01 | 0.32 | 0.50 | 0.18 |
| 13 | 0.24 | 0.78 | 0.54 | 0.48 | 2.29 | 1.81 |
| 14 | 0.05 | 0.03 | −0.02 | 0.14 | 0.14 | −0.01 |

An advantage of using CD137 to detect HSV-1-reactive CD8 cells is the ability to sort and expand these cells for downstream testing. Sorted, polyclonal CD3+ CD8+ CD137^{high} cells and control CD3+ CD8+ CD137^{low} cells were expanded with a non-specific mitogen. Resultant bulk populations were tested using autologous APC infected by HSV-1. Significant proportions of the sorted, expanded CD137^{high} cells selectively recognized the infected APC (representative data for participant 1 after two cell expansions in FIG. 1B; all persons in Table 3), while the CD137^{low} cells were non-reactive. Cytotoxicity was used to assess the antiviral effector function of the sorted, expanded cells. Brisk, self-restricted killing was noted for most participants (Table 3).

TABLE 3
Recognition of HSV-1 by polyclonal, twice mitogen-expanded
CD3+CD8+ PBMC selected on the basis of CD137 expression
after cross-presentation of HSV-1 by moDC.

| | IFN-γ | | | | | | | |
|---|---|---|---|---|---|---|---|---|
| | expression in | | cytotoxicity | | | cytotoxicity | | |
| | response to | | against | | | against | | |
| | autologous | | autologous | | | allogeneic | | |
| | BLCL ± HSV | | BLCL ± HSV | | | BLCL ± HSV | | |
| | infection | | infection | | | infection | | |
| | | HSV- | | HSV- | HSV- | | HSV- | HSV- |
| person | mock | 1 | mock | 1 | 2 | mock | 1 | 2 |
| 1 | 0.5 | 45.6 | −1.5 | 52.8 | 1.5 | 9.7 | −1.3 | 8.5 |
| 2 | 0.2 | 4.4 | 2.7 | 16.5 | 0.8 | 10.2 | 4.9 | 4 |
| 3 | 0.4 | 4.6 | 1.6 | 46.1 | 2.4 | 4.3 | 3.6 | 5.6 |
| 4 | 0.4 | 14.8 | −0.6 | 53.9 | 0.2 | 2.7 | 3.4 | 3.6 |
| 5 | 0.3 | 4.4 | −0.2 | 8.9 | −2.8 | 14.8 | 0.4 | −3.4 |
| 6 | 0.5 | 16.1 | 5.8 | 16.8 | −4.3 | −3.2 | 8.1 | −0.2 |
| 7 | 2.4 | 8.4 | 1.8 | 55.9 | 9.2 | 26 | 1.6 | 8.3 |

Detection and HLA Restriction of HSV-1 Antigen-Specific CD8 T-Cells.

We cryopreserved >10⁸ responder cells/person and interrogated their responses with panels of artificial APC (aAPC), expressing one of the participant's HLA A, B, or C molecules, and specific fragments of HSV-1 genetic material. Full length genes or fragments, together covering a total of 74 HSV-1 open reading frames (ORFs) were cloned into a custom vector suitable for expression in aAPC. Transfection efficiency for HLA class I was typically 5-20% at 48 h. Transfection efficiencies for the HSV-1 constructs were typically at least 10% as monitored with EGFP.

We completed CD8 ORFeome scans for every HLA A, B, and C in 7 HSV-1-infected persons (Table 1). T-cell activation was monitored by IFN-γ secretion after addition of polyclonal CD8 responders to the co-transfected aAPC. Due to near-homozygosity in one participant per locus, some participants had one scan at HLA A, B, or C. We used 21 distinct HLA class I cDNA molecules in all. The most frequently studied alleles were A*0201 (4 persons) and A*0101, B*4402, and Cw0701 (3 persons each).

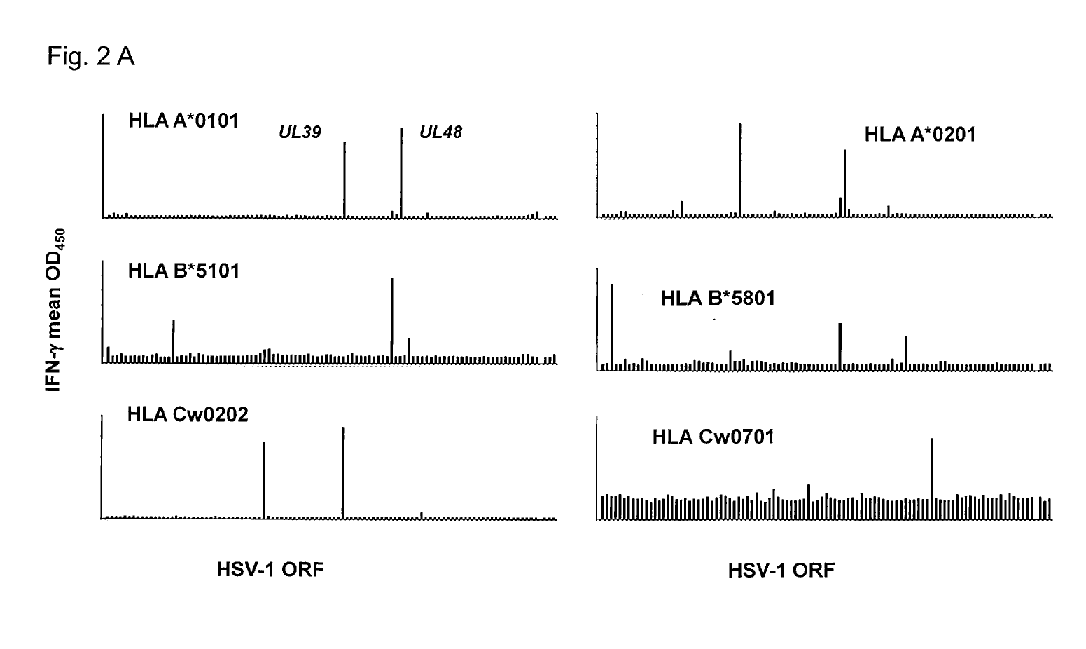

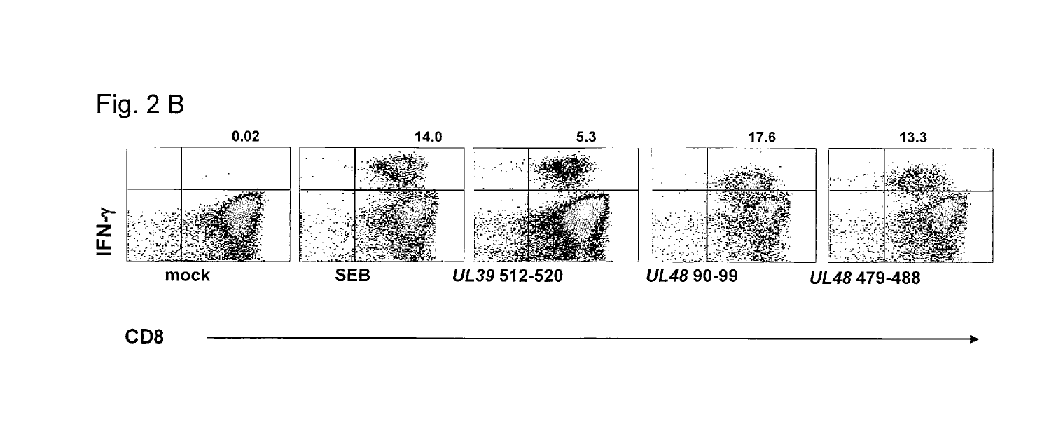

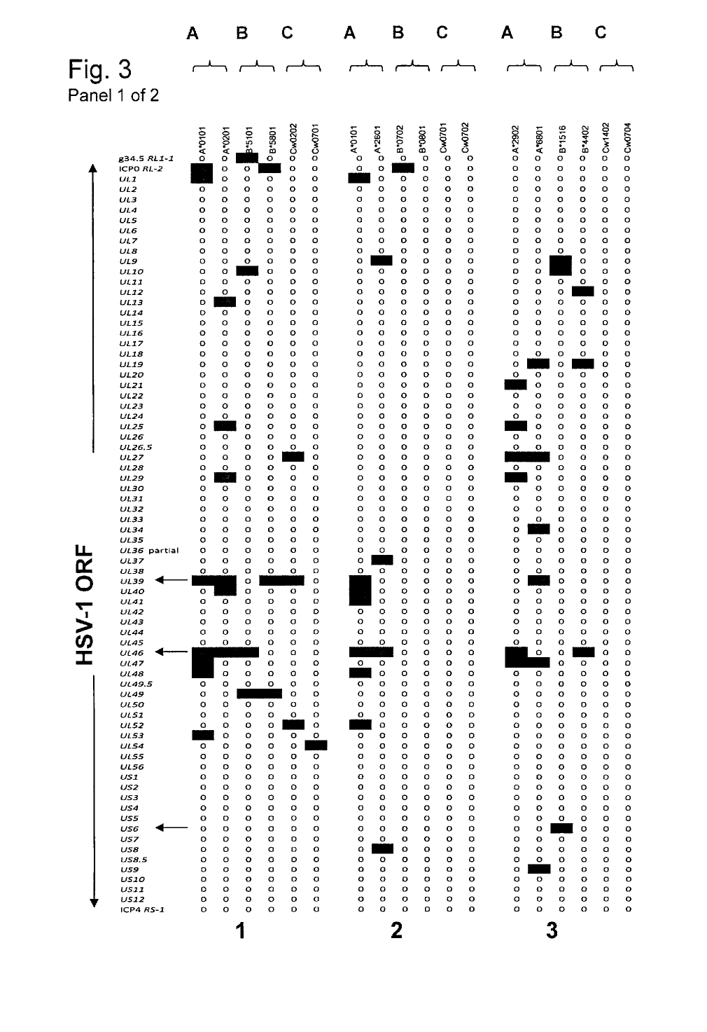

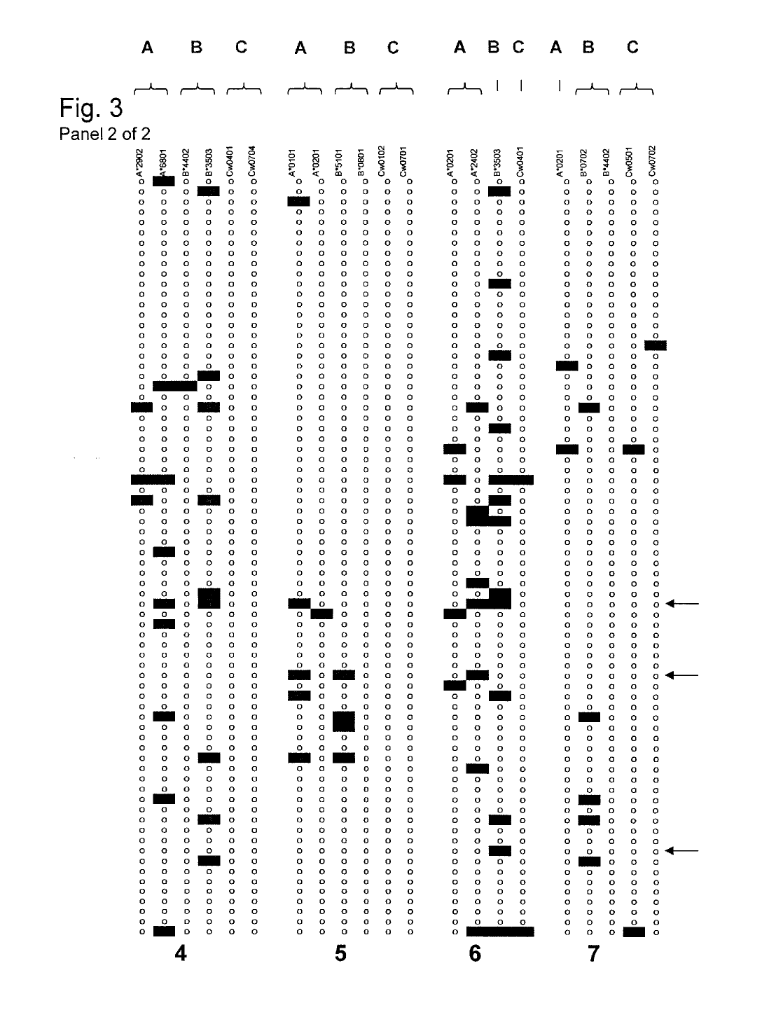

We observed discrete IFN-γ responses with very low backgrounds. A representative set of genome-wide screens for participant 1 (FIG. 2A) showed that each HLA allele had discrete CD8 reactivities. For example genes UL39 and UL48 had strong reactivity restricted by HLA A*0101, confirmed with synthetic peptides including two distinct epitopes in the UL48-endoded protein (FIG. 2B). Overall, there were 122 reactive combinations of HLA class 1 molecules and HSV-1 ORFs (FIG. 3). The number of CD8 ORF-level hits per person ranged from 10 to 27, with a median of 17 and mean±standard deviation of 17±7. Amongst these CD8 ORF-level hits, HLA A was the most frequently used locus and HLA C the least, with a mean±standard deviation of 10±4.0, 6.6±4.0, and 1.3±1.7 CD8 ORF-level hits at the A, B, and C loci, respectively. All six HLA A alleles and all six of seven HLA B alleles had one or more responses, while three of the eight HLA C alleles (0102, 0402, and 0704) did not. Overall, 28 of the 39 (72%) genome-wide screens yielded one or more reactive HSV-1 ORFs, with a range from zero to a maximum of 13 for HLA A*3503 in participant 6, and a mean±standard deviation of 5.2±2.3, 3.5±3.8, and 0.6±1.0 CD8 ORF-level hits in individual HLA A, B, and C allele screens, respectively.

HSV-1 ORFs Eliciting CD8 Responses Enriched by Cross-Presentation.

Overall, 40 distinct HSV-1 polypeptides were found to elicit CD8 IFN-γ responses amongst the 7 participants (UL1, UL9, UL10, UL12, UL13, UL15, UL16, UL17, UL18, UL19, UL21, UL23, UL25, UL27, UL29, UL30, UL31, UL34, UL37, UL38, UL39, UL40, UL41, UL46, UL47, UL48, UL49, UL50, UL52, UL53, UL54, US1/1.5, US3, US6, US7, USB, US9, RL2/ICP0, RL1, and RS1/ICP4). This represents 54% of the 74 unique proteins studied (FIG. 3). These proteins had diverse expression kinetic, structural, and functional roles as detailed in the literature (reviewed in (1)). Each immediate early protein expressed with a kinetics was represented, except for ICP47, a short polypeptide, as were early, leaky late, and true late polypeptides. Non-structural proteins and structural proteins in the capsid, tegument, and envelope were all targeted by CD8 T-cells. The most population prevalent CD8 antigen was encoded by UL39. This 1,137 amino acid long polypeptide is the large subunit of ribonucleotide reductase, is not detected in virions, and is a virulence factor in vivo but dispensable in vitro. UL39 was recognized in 11 of the 39 screens, in 6 of the 7 persons, and was restricted by 7 distinct HLA alleles: A*0101, A*0201, A*2402, A*6801, B*3503, B*5801, and Cw0202. The next most prevalently recognized protein was encoded by the abundant virion structural protein VP11/12, which is encoded by UL46. This protein is non-essential in vitro (1). UL46 was recognized in 10 screens and 5 participants, and also restricted by 7 distinct alleles: A*0101, A*0201, A*2402, A*2601, A*2902, B*4402, and B*5101.

CD8 responses to HSV-1 proteins gD1 and gB1 were of special interest because they share highly sequence homology to HSV-2 proteins that have been used as vaccine candidates (2, 43-45). HLA A*0201-restricted CD8 epitopes have been previously reported in both HSV-1 and HSV-2 (16, 17). Both proteins were well expressed in the pEXP103 system. Two of 39 HLA-level screens showed reactivity with gD1 (FIG. 3), for either HLA B*1516 or HLA B*3503. Amongst the four persons with HLA A*0201 or a close variant, none had A*0201-restricted responses to gD1. We additionally tested polyclonal HSV-reactive CD8 T-cell lines from these persons with three previously reported (16) A*0201-restricted synthetic peptides in gD that are sequence-identical in HSV-1 and HSV-2. Responses were not detected. In contrast, we demonstrated CD8 recognition of gB1 by six distinct HLA alleles and in 4 of 7 persons.

Definition of HSV-1 Peptide Epitopes Recognized by CD8 T-Cells Enriched by Cross-Presentation.

To test for the presence of discrete peptide epitopes, we selected a subset of the CD8 antigens (FIG. 3) that used HLA alleles with high population prevalence and had well-developed peptide binding prediction algorithms. We tested peptides from 24 distinct HSV-1 ORFs for potential HLA A*0101, A*0201, A*2402, A*2902, or B*0702-restricted peptide epitopes. Bulk CD137^{high}-selected CD8 cells were tested for IFN-γ expression by intracellular cytokine cytometry (ICC) using autologous PBMC as APC. DMSO negative control gave very low background, such that tight groupings of IFN-γ (+) CD8 T-cells could be discerned for antigenic peptides (representative data, FIG. 2B). Overlapping reactive 9 and 10-mer peptides were tallied as a single novel epitope. Overall, we defined 45 distinct HSV-1 CD8 distinct epitopes (Table 4) in 22 ORFs. Several reactive ORFs had more than one constituent CD8 peptide epitope (Table 4).

TABLE 4
HSV-1 CD8 epitopes and HSV-2 homologs. Polyclonal responder cells
derived by cross-presentation and positive when screened with the indicated
HLA alleles and ORFs were reactive with the indicated HSV-1 peptides (SEQ
ID NOs: 1-45).

| HLA | ORF^{b} | HSV-1 AA^{c} | HSV-1 | HSV-2 | HSV-2 AA^{d} | TC/TS^{d} |
|---|---|---|---|---|---|---|
| A*0101 | UL1 | 66-74 | LIDGIFLRY | R-------- | 66-74 | TS |
| A*0101 | UL39 | 512-520^{e} | YMESVFQMY | --------- | 517-525 | TC |
| A*0101 | UL41 | 259-268 | HTDLHPNNTY | ---------- | 261-270 | TC |
| A*0101 | UL46 | 354-362^{e} | ATDSLNNEY | -S------- | 354-362 | TS |
| A*0101 | UL47 | 360-368 | RSSLGSLLY | --------- | 363-371 | TC |
| A*0101 | UL47 | 566-574 | FTAPEVGTY | S-------- | 569-577 | TS |
| A*0101 | UL48 | 90-99 | SALPTNADLY | -GF-----M- | 88-97 | TS |
| A*0101 | UL48 | 479-488 | FTDALGIDEY | ----M---DF | 479-488 | TS |
| A*0101 | UL53 | 201-209 | ETDPVTFLY | -A-------- | 201-209 | TS |
| A*0201 | UL13 | 389-397 | TLLELVVSV | -----L--- | 389-397 | TS |
| A*0201 | UL25 | 367-375^{e} | FLWEDQTLL | --------- | 372-380 | TC |
| A*0201 | UL27 | 280-288 | SVYPYDEFV | --------- | 275-283 | TC |
| A*0201 | UL27 | 448-456^{e} | FLIAYQPLL | --------- | 443-451 | TC |
| A*0201 | UL39 | 425-433 | RILGVLVHL | --------- | 430-438 | TC |
| A*0201 | UL40 | 184-192^{f} | ILIEGIFFA | -----V--- | 181-189 | TS |
| A*0201 | UL47 | 286-294 | FLADAVVRL | --V--I--V | 289-297 | TS |
| A*0201 | UL47 | 374-382 | ALLDRDCRV | ---G----- | 377-385 | TS |
| A*0201 | UL47 | 545-553 | RLLGFADTV | ----L---- | 548-556 | TS |
| A*2402 | UL21 | 162-170^{f} | VYTPSPYVF | A-------- | 162-170 | TS |
| A*2402 | UL31 | 292-300 | EYQRLYATF | --------- | 291-299 | TC |
| A*2402 | UL37 | 221-230 | AYSLLFPAPF | -------S-I | 221-230 | TS |
| A*2402 | UL37 | 640-648 | AYLPRPVEF | ------I-- | 640-648 | TS |
| A*2402 | UL46 | 226-234 | AYVSVLYRW | -------H- | 226-234 | TS |
| A*2402 | UL54 | 504-512 | KYFYCNSLF | --------- | 504-512 | TC |
| A*2402 | ICP4 | 1097- | LYPDAPPLRL | PDG-----V- | 1243- | TS |
| A*2902 | UL25 | 170-179 | SSGVVFGTWY | ---------- | 175-184 | TC |
| A*2902 | UL25 | 235-243 | AVLCLYLLY | --------- | 240-249 | TC |
| A*2902 | UL26 | 22-30 | YVAGFLALY | --------- | 22-30 | TC |
| A*2902 | UL26 | 326-334^{f} | YLWIPASHY | --------- | 328-336 | TC |
| A*2902 | UL27 | 295-303 | VYMSPFYGY | --------- | 290-298 | TC |
| A*2902 | UL27 | 641-649 | FTFGGGYVY | -I------- | 638-646 | TS |
| A*2902 | UL29 | 460-468 | ALLAKMLFY | --------- | 460-468 | TC |
| A*2902 | UL29 | 895-903^{f} | YMANQILRY | --------- | 895-903 | TC |
| A*2902 | UL46 | 93-101 | LASDPHYEY | -------D- | 93-101 | TS |
| A*2902 | UL46 | 126-134 | AILTQYWKY | ---A----- | 126-134 | TS |
| A*2902 | UL46 | 224-232 | LLAYVSVLY | --------- | 224-232 | TC |
| A*2902 | UL46 | 333-341 | SIVHHHAQY | --------- | 333-341 | TC |
| A*2902 | UL47 | 508-516^{f} | ALATVTLKY | --------- | 511-519 | TC |
| B*0702 | ICPO | 698-706^{e} | VPGWSRRTL | A-A------ | 742-750 | TS |
| B*0702 | UL21 | 382-390 | VPRPDDPVL | --A--E-T- | 380-388 | TS |
| B*0702 | UL49 | 281-290 | RPTERPRAPA | --AG-AA-T- | 276-285 | TS |
| B*0702 | US1 | 70-78 | APRIGGRRA | GDLR----R | 62-70 | TS |
| B*0702 | US7 | 22-30 | VVRGPTVSL | --------- | 22-30 | TC |
| B*0702 | US7 | 97-105 | CPRRPAVAF | --------- | 97-105 | TC |
| B*0702 | US7 | 195-203 | APASVYQPA | G-S---T-G | 195-203 | TS |

^{a} HLA cDNA used to screen HSV-1 ORFeome clone set.
^{b}HSV-1 ORF scoring positive that yielded a reactive peptide.
^{c}Amino acid numbers and HSV-1 sequences from Genbank NC_001806.1 scoring positive in IFN-γ ICC assay.
^{d}Predicted amino acid sequence and homologous portion of corresponding HSV-2 protein from Genbank NC_001798.1 (HSV-2). TC = type common epitope, identical between HSV-1 and HSV-2; TS = type specific.
^{e}The HSV-2 homologs of these epitopes were previously described as CD8 epitopes with the same proven or probable HLA restriction using PBMC from HSV-2-infected persons and HSV-2 peptides (52-54). In the case of ICP0 742-750 of HSV-2, the epitope was previously assigned amino acids 743-751 (93) based on our finding of an extra amino acid at the exon 1-exon 2 splice junction, based on cDNA sequencing, that is not present in Genbank NC_001798.1.
^{f}Multiple synthetic peptides were positive. For HLA A*0201/UL40, 9-mer 184-192 and 10-mers 184-193 and 183-192 were positive. Similarly, for HLA A*2902/UL26, 10-mer 325-334 and 9-mer 326-334 were positive, for HLA A*2902/UL29, 10-mer 894-903 and 9-mer 895-903 were positive, for HLA A*2902/UL47, 10-mer 508-517 and 9-mer 508-516 were positive, and for HLA A*2402/UL21, 10 mer 161-170 and 9-mer 162-170 were positive.

CD8 Responses in Direct PBMC Assays.

The CD8 T-cell line data above are qualitative rather than quantitative. The enrichment afforded by cross-presentation and cell selection could detect responses that were below the limit of detection in direct PBMC samples, and there is also the possibility we were detecting in vitro priming by moDC. Epitope-specific responses in direct PBMC assays cannot represent in vitro priming, and are amenable to detailed phenotypic studies using tetramers and ICC. We therefore used IFN-γ ELISPOT to survey and rank epitope-specific responses for further studies. PBMC from 20 HLA-appropriate persons with HSV-1 infection were matched with CD8 peptide epitopes with known HLA restriction. We tested 40 of the 45 CD8 epitopes (Table 4) as detailed in Methods. The participants included the discovery cohort (Table 1) and 13 additional HSV-1 infected persons. Each person had a strong IFN-γ response to whole UV HSV-1 antigen (likely CD4 cells) and no spot-forming units for DMSO negative control.

Among 256 HLA-matched combinations of PBMC and HSV-1 peptides in the overall set, 23 (9.0%) were positive (red cells with integers in FIG. 4). 12 of 20 (60%) persons had from one to five peptide-specific responses in direct PBMC testing. Amongst the 40 CD8 peptide epitopes tested, 12 (30%) were positive in one or more HLA-matched persons. The number of net IFN-γ ELISPOT (+) cells ranged from 11 to 136 SFU/10⁶ PBMC. The highest rate of positivity was for the HLA A*0201 peptides, with 6 of 9 epitopes positive in at least one person and two persons positive for 4 distinct A*0201 peptides. The “hits” in the HLA-ORF screens and confirmed by direct PBMC testing are biologically present above the threshold of the direct ELISPOT test. Some persons with positive ORF-level responses in HLA-specific screens (red in FIG. 3) did not react with the peptides tested (blue in FIG. 4). Peptide-reactive cells could be present below the limit of detection ex vivo. Alternatively, one or more undiscovered HLA-restricted epitopes account for the ORF-level positive responses (FIG. 3).

HSV-1 ORFs Stimulating Population-Prevalent CD4 Responses.

As noted above, measurement of un-manipulated PBMC is limited by the low integrated frequency of HSV-1-reactive CD4 cells and the need for highly purified recombinant proteins or a very large peptide set. We therefore enriched and expanded HSV-1-reactive CD4 T-cells using protocols designed to yield large numbers of polyclonal responder cells. The initial stimulation used whole, cell-associated, UV-killed HSV-1, a format previously shown to re-stimulate CD4 T-cells specific for a variety of structural and non-structural proteins in the context of HSV-2 (46). After 20 h, a small percentage of live, CD3+ CD4+ cells in PBMC specifically expressed CD137 (representative participant, FIG. 5A). Cell lines created by sort-purification and expansion with non-specific mitogens were highly enriched for HSV-1-reactive CD4 cells, while sorted CD137^{low} cells were non-reactive (FIG. 5A shows data after two cell expansions; enrichment was similar after the first expansion).

We expressed proteins covering the large majority of the HSV-1 proteome using an in vitro bacterial expression system. Anti-6-His IB showed specific staining. We titered the ORFeome set in preliminary proliferation assays and found that a dilution of 1:5,000 gave optimal responses with low background. We further checked potency and identity with a CD4 T-cell clone specific for HSV-1 protein VP22 (gene UL49) (18) and noted strong, specific responses.

Polyclonal CD137^{high} CD4 cell lines showed discrete patterns of reactivity when assayed against the protein set (representative participant, FIG. 5B) in each of eleven persons, including those with detailed CD8 studies (Table 1). The average number of HSV-1 ORFs recognized per individual was 22.8±7.0 (mean±SD). Overall, 52 of the 74 (70%) of the unique HSV-1 polypeptides were recognized at least once. On a population basis, the most prevalent CD4 responses were noted for envelope glycoproteins gB1 and gD1, and tegument protein VP11/12 encoded by gene UL46, discussed above for CD8 cells. Each was recognized by 11 of 11 persons (100%). Inspection of the overlap between CD8 and CD4 antigens (FIG. 5C) shows that the proteins encoded by UL39 and UL46 were frequently recognized. Grouping of HSV-1 proteins by their kinetics of expression during viral replication (1) indicates that true-late proteins are less commonly recognized by CD8 cells than are proteins in other classes.

Application to Vaccinia Virus.

Participant 9 from a previous report (47) was re-vaccinated with vaccinia 20 months prior to phlebotomy. Using cross-presentation for CD8 T-cells, followed by CD137 detection outlined exactly as done for HSV-1, we detected low but specific CD8 reactivity at 20 h. Selected, expanded CD137^{high} but not CD137^{low} cells were highly enriched in vaccinia virus-specific CD8 T-cells and these cells had specific cytotoxicity. Participant 8 (47) who had been re-vaccinated 40 months prior to study was studied for CD4 responses. We used UV-inactivated whole vaccinia virus and CD137-based selection, again as detailed for HSV-1. The CD137^{high}-origin bulk cells were very highly enriched for T-cells making IFN-γ with or without IL-2 in response to vaccinia, while these were essentially absent from CD137^{low} cells. All cell populations responded to the positive control PMA/ionomycin stimulation, albeit with varying cytokine patterns. Similar results were obtained for several vaccinia-immune persons. The CD8 and CD4 T-cell enrichment methods (outlined in FIG. 6) are thus applicable to two large-genome microbes. Downstream CD4 analyses showed responses to discrete vaccinia ORFs.

### Discussion

HSV-1 is an important human pathogen, but there are no vaccines in active clinical development. In this report, we have shown that the proteins encoded by HSV-1 genes UL39 and UL46 have coordinated CD8 and CD4 immunogenicity in most persons and are therefore rational vaccine candidates. We have provided an estimate of the complexity of the response, documented clustering of responses based on HLA type, and defined myriad novel CD8 antigens and epitopes. We have identified a new hierarchy of responses based on HLA locus, with HLA A more frequently responsible for antigen presentation than HLA B, and HLA C having a minor contribution. Parallel studies determined that infected humans recognize a mean of 17 and 23 HSV-1 ORFs as CD8 and CD4 antigens, respectively. With the exceptions noted in the Introduction and Results, the vast majority of the antigenic reactivities and epitopes we have defined are novel. We also showed applicability to another large-genome virus and anticipate that these systems may be useful for many pathogens.

T-cell responses to pathogens have been difficult to access for several reasons. Microbe-specific T-cells can occur at low abundance in the blood, such that an unbiased pre-enrichment step is helpful for new antigen or epitope discovery. Sylwester et al.'s probe of the response to the CMV proteome using peptides and direct PBMC ICC was enabled by the high overall abundance of T-cell responses to CMV (48). Responses to EBV are also large, such that a direct PBMC approach to CD8 responses using a cloned partial ORFeome has yielded hits (49). Indeed, the low magnitude of direct PBMC IFN-γ ELISPOT responses to single HSV-1 peptides in the current report contrast with the high magnitude responses noted to single epitopes in CMV and EBV (48, 50).

We have shown that re-stimulation methods tailored for CD8 or CD4 T-cells, based on the biology of antigen presentation, can be used without change for two distinct viruses. Once responder cells are enriched and expanded, the large microbial genomes still harbor myriad potential epitopes that are challenging to decode. In this report, we first assigned CD8 reactivity at the level of HSV-1 ORFs, using efficient plasmid sets. Limited use of peptide binding algorithms and peptide synthesis efficiently confirmed antigenicity to the epitope level. The new, validated peptide reagents were in turn positive in a proportion of additional HLA-appropriate subjects in direct ex vivo assays. Even in our limited study population, CD8 T-cells from discrete persons frequently had reactivity, in our primary co-transfection screens, with the same HSV-1 ORFs when studied using shared HLA cDNA molecules. For example, each HLA A*0101-bearing donor had CD8 T cells recognizing the HSV-1 ORFs UL 1 and UL48 (FIG. 3). Clustering studies to measure this tendency will be the subject of future analyses.

In addition to identifying UL39 and UL46 as rational candidate subunit vaccines, our data have implications for the design of whole virus-formats. In fact, the diversity of the CD8 response, with an average of 17 antigens per person, implies that a whole virus rather than a subunit approach to vaccination for HSV-1 is most likely to mimic the immune response to natural infection. Globally, we found that broad kinetic and structural and functional spectra of HSV-1 proteins were recognized by the human CD8 response. Therefore, either replication-incompetent or attenuated replication-competent vaccine formats have the potential to stimulate broad CD8 responses in most persons. Amongst whole HSV vaccine candidates that replicate discontinuously in normal cells, those that allow expression to proceed relatively completely may be quite rational (25, 26). To explore the virological rules of HSV-1 T-cell antigenicity, we scored each protein dichotomously for CD8 responses in the study population, and then categorized each protein studied as immediate early, early, late but not otherwise characterized, early-late with synthesis prior to DNA replication, or true-late with expression only after DNA replication (1). We also recorded, for each HSV-1 protein, its absence, detection at <1%, or detection at >1% of adjusted virion mass using mass spectroscopy data of purified virions (51). These analyses disclosed a weighting of CD8 responses towards HSV-1 proteins expressed prior to HSV-1 DNA replication, and towards abundant virion polypeptides. Specifically, only 3 of 17 true-late proteins (18%) were recognized by CD8 cells. In contrast, 4 of 5 immediate-early proteins (80%), 9 of 12 early proteins (75%), 12 of 19 early-late proteins (63%), 12 of 20 late proteins not specified as early or late (60%), and 0 of 1 proteins with no specified expression kinetics (0%) were CD8 antigens. Amongst the more abundant virion proteins, 17 of 23 (74%) were positive for CD8 responses. In contrast, only 23 of 51 (45%) proteins either absent from virions or present at <1% levels were CD8 antigens. The population prevalence for CD4 responses did not segregate by HSV-1 kinetic or structural class. Our data suggest that whole-virus HSV-1 format vaccines which express most proteins normally made prior to DNA synthesis, or which can be dosed to provide a large mass of virion input proteins, should retain the potential to stimulate broad CD8 responses.

A vaccine covering HSV-1 and HSV-2 would be desirable. Half of the minimal HSV-1 CD8 epitopes newly defined in this report are sequence-identical in HSV-1 and HSV-2, and appropriate for candidate type-common vaccines. Indeed, the HSV-2 homologs of three epitopes, HLA A*0101/HSV-1 UL39 512-520, HLA A*0201/HSV-1 UL25 367-375, and HLA A*0201/HSV-1 UL27 448-456, were found in our prior HSV-2 work (52-54). HSV CD8 epitopes can also tolerate amino acid substitutions, as exemplified by UL46 354-362 of HSV-1 and HSV-2, differing at amino acid two, and by ICP0 HSV-1 698-706 and its' homolog HSV-2 ICP0 742-750, differing at amino acids one and three. It is certainly possible that cross-reactive T-cells could be involved in cross-protection against some aspects of HSV-2 infection or severity observed in HSV-1 infected persons (55). Most of our subjects were dually infected with both HSV types. Future cross-sectional studies comparing immune responses to HSV-1 in the presence or absence of HSV-2 co-infection can clarify the extent to which each infection contributes to the cross-reactive repertoire.

With regards to effector function, we showed that bulk HSV-specific CD8 T-cells enriched using cross-presentation and CD137 have brisk virus-specific cytotoxicity. We plan to enrich peptide-specific CD8 cells with peptides (56) or tetramers (57) and study recognition of HSV-1-infected skin cells to more closely mimic physiologic target cells. The Cos-7 system has not been characterized for use in CTL assays, so we will move to a viral infection system. In HSV-2 studies, T-cells recognizing diverse antigens were able to lyse HSV-infected skin cells, but the specific conditions, such as the dose and time of infection, and the requirement for de novo viral protein synthesis or for IFN-γ pre-treatment (56, 58), differed between epitopes. With the larger panel of HSV-1 epitopes we hope to establish general rules for CD8 recognition of physiologically relevant cells which could inform vaccinology. Future cross-sectional study of populations with defined levels of HSV-1 severity, and longitudinal research during the ontogeny of primary immune responses or during reactivations in the chronic phase may also contribute correlates of severity and reactivation that could further influence vaccine design.

Interestingly, UL39 was a strong HSV-1 CD8 antigen in both humans and the one mouse MHC haplotype studied, H-2^{b}. UL39 is a virulence factor involved in evading innate immunity and apoptosis (59), such that immune targeting of UL39 may be advantageous to the host. The CD8 repertoire in infected C57BL/6 mice had a breadth of 19 HSV-1 epitopes. These were concentrated in only three ORFs, gB1 (gene UL27), UL39, and ICP8 (gene UL29) (60). The mice did not recognize immediate-early HSV-1 polypeptides, while responses to ICP0, ICP4, ICP22, and ICP47 were detected in humans. In addition to MHC class I-peptide binding preferences, these differences may reflect species-specific effects of HSV-1 HLA class I immune evasion genes (61) and the fact that the human exposure to HSV-1 antigen is chronic and intermittent, while HSV-1 typically does not recur in mice. We conclude that study of adaptive immunity in the natural host is a necessary counterpoint to the powerful manipulative experiments that are possible in experiment models of HSV-1 infection.

Several factors may have influenced our results. Our CD8 workflow used cross-presentation at the re-stimulation step, but then switched to direct present by aAPC or peptide-loaded cells at the readout step. We observed that cross-presentation is efficient in presenting diverse HSV-1 proteins to CD8 T-cells. The HSV proteins ICP47 (gene US12) and vhs (gene UL41) inhibit direct presentation (30). We have previously re-stimulated memory CD8 cells using direct presentation by HSV-infected B-LCL, but this yielded a paucity of HSV-reactive CD8 clones reactive only with membrane glycoproteins (62). HSV-infected fibroblasts failed in this endeavor. Direct presentation by infected DC might uncover epitopes specific for this pathway, albeit HSV infection harms various DC and renders them defective for antigen presentation (63-65). Future studies will compare direct and cross-presentation at the re-stimulation stage.

Expression of the HSV-1 proteome was not totally complete. Genes UL15.5, UL20.5, UL27.5, and UL43.5 are under development, as is the C-terminal ˜500 amino acids of the UL36 protein. Genes predicted to be in-frame subsets of longer polypeptides were not included but this will not lead to loss of potential epitopes. A poorly studied variable is allelic heterogeneity in HLA class I assembly with Chlorocebus sp. β2m in Cos-7 cells. We over-expressed HSV-1 ORFs in isolation in aAPC, where intracellular trafficking and class I presentation could differ from the viral context. There were subtle differences in some of our HLA C constructs from the HLA and B vectors, but our method have achieved excellent HLA C expression (66). There are interactions between HSV-1 proteins such as proteolysis and phosphorylation (1), and possibly species-specific host protein-HSV-1 protein interactions, that would differ between infected and transfected cells. In HSV-2 work using a genomic DNA library and Cos-7 transfection, we decoded the fine specificity of each CD8 clone studied (52, 56, 67) and therefore believe such situations are rare for HSV. We focused on IFN-γ readouts of CD8 T-cell activation, and proliferation to detect CD4 T-cell responses. With regards to effector cytokines, rare HSV-reactive T-cells in PBMC making TNF-α or IL-2 but not IFN-γ have been described (54). In preliminary studies, substitution of TNF-α for IFN-γ ELISA did not uncover additional specificities. We noted one HSV-seronegative person with CD8 responses to HSV-1 (participant 13 in Table 2). Further work will be required to determine if reactivity can be confirmed at the epitope level, as has been done for HSV-2 (68).

We have extended the use of CD137 as an activation marker to two complex microbes for both CD4 and CD8 T-cells. CD137 mediates a strong co-stimulatory signal to T-cells. Thus, use of anti-CD137 to detect and purify antigen-reactive cells may assist their downstream expansion. Our data are consistent with some level of bystander CD137 expression, as the level of reactivity with whole HSV-1 amongst expanded CD137^{high} cells varied between 4% and 45%. Enrichment was better for CD4 cells. We cannot be sure that all memory HSV-reactive cells up-regulated CD137. CD137 is similar in this regard to other molecules used for enrichment such as CD134, CD154, and captured IFN-γ (69).

In summary, the T-cell response to a complex and serious pathogen with a large genome, HSV-1, has been decoded with a linked set of cellular and molecular tools to reveal novel candidate vaccine antigens. We have identified the proteins encoded by genes UL39 and UL46 as having high population prevalence of coordinated CD8 and CD4 responses. Cross-presentation followed by CD137-based selection also effectively enriches rare CD8 cells specific for vaccinia virus, an effective live virus vaccine. CD137 is also suitable for enrichment of CD4 T-cells reactive with whole microbe preparations, as demonstrated for both HSV-1 and vaccinia virus. Our flexible gene cloning format allows integrated, efficient study of both CD8 and CD4 responses after one round of PCR-based cloning of microbial ORFs. Use of appropriate initial re-stimulation conditions, CD137 as a flexible selection marker, and the genomes and complete genome-covering ORF sets now available for Mycobacterium tuberculosis, Plasmodium falciparum, and other agents should speed comprehensive definition of T-cell responses and vaccine design.

### REFERENCES

1. Roizman, B., et al. 2007. Herpes simplex viruses. In Fields Virology. D. M. Knipe, Howley, P. M., editor. Philadelphia: Lippincott, Williams, and Wilkins. 2501-2602.
2. Stanberry, L. R., et al. 2002. New England Journal of Medicine 347:1652-1661.
3. Verjans, G. M. G. M., et al. 2007. Proc Natl Acad Sci USA 104:3496-3501.
4. Jing L, V. G., et al. 2009. In Immunology 2009: 96th Annual Meeting of the American Association of Immunologists. Seattle. Abstract 128.124, page 172.
5. Prabhakaran, K., et al. 2005. Immunity 23:515-525.
6. Zhu, J., et al. 2007. J Exp Med 204:595-603.
7. Zhu, J., et al. 2009. Nat Med 15:886-892.
8. Gebhardt, T., et al. 2009. Nat Immunol 10:524-530.
9. Sawtell, N. M. 1998. Journal of Virology 72:6888-6892.
10. Sawtell, N. M., et al. 2001. Journal of Infectious Diseases 184:964-971.
11. Orr, M. T., et al. 2007. J Immunol 178:4731-4735.
12. Iijima, N., et al. 2008. J Exp Med 205:3041-3052.
13. Schacker, T., et al. 1998. Journal of Infectious Diseases 178:1616-1622.
14. McGeoch, D. J., et al. 1986. Nucleic Acids Research 14:1727-1745.
15. McGeoch, D. J., et al. 1988. Journal of General Virology 69:1531-1574.
16. Chentoufi, A. A., et al. 2008. J Immunol 180:426-437.
17. Chentoufi, A. A., et al. 2010. J Immunol 184:2561-2571.
18. Koelle, D. M., et al. 2000. J Virol 74:10930-10938.
19. Verjans, G. M. G. M., et al. 1998. Journal of Infectious Diseases 177:484-488.
20. Verjans, G. M. G. M., et al. 2000. Investigative Ophthalmology and Visual Science 41:2607-2612.
21. Remeijer, L., et al. 2004. Ocul Immunol Inflamm 12:255-285.
22. Verjans, G. M., et al. 2000. Journal of Infectious Diseases 182:923-927.
23. Verjans, G. M., et al. 1998. Journal of Infectious Diseases 178:27-34.
24. Yasukawa, M., and Zarling, J. M. 1985. Journal of Immunology 134:2679-2687.
25. Hoshino, Y., et al. 2005. J Virol 79:410-418.
26. de Bruyn, G., et al. 2006. Vaccine 24:914-920.
27. Aurelian, L., et al. 1999. Vaccine 9:1951-1963.
28. York, I. A., et al. 1994. Cell 77:525-535.
29. Hill, A., et al. 1995. Nature 375:411-415.
30. Tigges, M. A., et al. 1996. Journal of Immunology 156:3901-3910.
31. Posavad, C. M., et al. 1993. Journal of Immunology 151:4865-4873.
32. Sloan, D. D., et al. 2006. J Immunol 176:1825-1833.
33. Jirmo, A. C., et al. 2009. J Immunol 182:283-292.
34. Smith, C. M., et al. 2003. J Immunol 170:4437-4440.
35. Allan, R. S., et al. 2003. Science 301:1925-1928.
36. Allan, R. S., et al. 2006. Immunity 25:153-162.
37. Wakim, L. M., et al. 2008. Science 319:198-202.
38. Bedoui, S., et al. 2009. Nat Immunol 10:488-495.
39. Bosnjak, L., et al. 2005. J Immunol 174:2220-2227.
40. Wolfl, M., et al. 2008. Cytometry A 73:1043-1049.
41. Wehler, T. C., et al. 2008. J Immunol Methods 339:23-37.
42. Watanabe, K., et al. 2008. Int J Hematol 88:311-320.
43. Corey, L., et al. 1999. Journal of the American Medical Association 282:331-340.
44. Straus, S. E., et al. 1994. Lancet 343:1460-1463.
45. Straus, S. E., et al. 1997. Journal of Infectious Diseases 176:1129-1134.
46. Koelle, D. M., et al. 2000. Journal of Virology 74:11422-11425.
47. Jing, L., et al. 2008. J. Virol. 82: 7120-7134.
48. Sylwester, A. W., et al. 2005. J Exp Med 202:673-685.
49. Saulquin, X., et al. 2000. European Journal of Immunology 30:2531-2539.
50. Hislop, A. D., et al. 2007. Annu Rev Immuno/25:587-617.
51. Loret, S., et al. 2008. J Virol 82:8605-8618.
52. Koelle, D. M., et al. 2003. Proc Natl Acad Sci USA 100:12899-12904.
53. Koelle, D. M., et al. 2008. Clin Vaccine Immunol 15:773-782.
54. Laing, K. J., et al L. 2010. J Clin Immunol.
55. Koelle, D. M., and Corey, L. 2008. Annu Rev Med 59:381-395.
56. Koelle, D. M., et al. 2001. J Immunol 166:4049-4058.
57. Dong, L., Li, P., et al. 2010. J. Immunol.
58. Cunningham, A. L., et al. 1985. Journal of Clinical Investigation 75:226-233.
59. Dufour, F., et al. 2011. Apoptosis 16:256-271.
60. St Leger, A. J., et al. 2011. J Immunol 186:3927-3933.
61. Tomazin, R., et al. 1998. Journal of Virology 72:2560-2563.
62. Tigges, M. A., et al. 1992. Journal of Virology 66:1622-1634.
63. Puttur, F. K., et al. 2010. J Immunol 185:477-487.
64. Jin, H., et al. 2011. J Virol 85:3397-3407.
65. Salio, M., et al. 1999. European Journal of Immunology 29:3245-3253.
66. Akatsuka, Y., et al. 2002. Tissue Antigens 59:502-511.
67. Koelle, D. M., et al. 2002. J Clin Invest 110:537-548.
68. Posavad, C. M., et al. 2003. Journal of Immunology 170:4380-4388.
69. Zaunders, J. J., et al. 2009. J Immunol 183:2827-2836.
70. Ashley, R. L., et al. 1988. J Clin Microbiol 26:662-667.
71. Jing, L., et al. 2005. J Immunol 175:7550-7559.
72. Koelle, D. M., et al. 1994. Journal of Virology 68:2803-2810.
73. Marsh, S. G., et al. 2010. Tissue Antigens 75:291-455.
74. Spruance, S. L., and Chow, F. S. 1980. Journal of Infectious Diseases 142:671-675.
75. Kit, S., et al. 1983. Biochim. Biophys. Acta 741:158-170.
76. Jing, C.-L., et al. 2005. In 43rd Annual Meeting of the Infectious Diseases Society of America (IDSA). San Francisco. abstract 1034, page 1227.
77. Jing, L., et al. 2007. J Immunol 178:6374-6386.
78. Yasukawa, M., and Zarling, J. M. 1983. Journal of Immunology 131:2011-2016.
79. Koelle, D. M., et al. 2001. Journal of Immunology 166:4049-4058.
80. Quakkelaar, E. D., et al. 2011. PLoS ONE 6:e16819.
81. Koelle, D. M. 2003. Methods 29:213-226.
82. Koelle, D. M., et al. 1998. Journal of Virology 72:7476-7483.
83. Kask, A. S., et al. 2010. Vaccine 28:7483-7491.
84. Koelle, D. M., et al. 2002. Blood 99:3844-3847.
85. Robinson J, M. A., et al. 2000. Tissue Antigens 55:28-287.
86. McMichael, A. J., et al. 1980. Human Immunology 1:121-129.
87. Jing, L., et al. 2008. J Virol 82:7120-7134.
88. Jing, L., et al. 2009. J Immunol Methods 347:36-45.
89. Eyles, J. E., et al. 2007. Proteomics 7:2172-2183.
90. Koelle, D. M., et al. 1998. Journal of Clinical Investigation 101:1500-1508.
91. Vita, R., et al. 2010. Nucleic Acids Res 38:D854-862.
92. Marsh, S. G. E., et al. 2000. The HLA FactsBook. San Diego: Academic Press. 398 pp.
93. Koelle, D. M., et al. 2002. Journal of Clinical Investigation 110:537-548.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A pharmaceutical composition comprising an isolated HSV-1 polypeptide, or an isolated polynucleotide encoding the polypeptide, wherein the polypeptide comprises an immunogenic portion of an HSV antigen, and, optionally, an adjuvant, wherein the immunogenic portion comprises one or more epitopes selected from the group consisting of: amino acids 66-74 of UL1 (LIDGIFLRY; SEQ ID NO: 1), amino acids 512-520 of UL39 (YMESVFQMY; SEQ ID NO: 2), amino acids 259-268 of UL41 (HTDLHPNNTY; SEQ ID NO: 3), amino acids 354-362 of UL46 (ATDSLNNEY; SEQ ID NO: 4), amino acids 360-368 of UL47 (RSSLGSLLY; SEQ ID NO: 5), amino acids 566-574 of UL47 (FTAPEVGTY; SEQ ID NO: 6), amino acids 90-99 of UL48 (SALPTNADLY; SEQ ID NO: 7), amino acids 479-488 of UL48 (FTDALGIDEY; SEQ ID NO: 8), amino acids 201-209 of UL53 (ETDPVTFLY; SEQ ID NO: 9), amino acids 389-397 of UL13 (TLLELVVSV; SEQ ID NO: 10), amino acids 367-375 of UL25 (FLWEDQTLL; SEQ ID NO: 11), amino acids 280-288 of UL27 (SVYPYDEFV; SEQ ID NO: 12), amino acids 448-456 of UL27 (FLIAYQPLL; SEQ ID NO: 13), amino acids 425-433 of UL39 (RILGVLVHL; SEQ ID NO: 14), amino acids 184-192 of UL40 (ILIEGIFFA; SEQ ID NO: 15), amino acids 286-294 of UL47 (FLADAVVRL; SEQ ID NO: 16), amino acids 374-382 of UL47 (ALLDRDCRV; SEQ ID NO: 17), amino acids 545-553 of UL47 (RLLGFADTV; SEQ ID NO: 18), amino acids 162-170 of UL21 (VYTPSPYVF; SEQ ID NO: 19), amino acids 292-300 of UL31 (EYQRLYATF; SEQ ID NO: 20), amino acids 221-230 of UL37 (AYSLLFPAPF; SEQ ID NO: 21), amino acids 640-648 of UL37 (AYLPRPVEF; SEQ ID NO: 22), amino acids 226-234 of UL46 (AYVSVLYRW; SEQ ID NO: 23), amino acids 504-512 of UL54 (KYFYCNSLF; SEQ ID NO: 24), amino acids 1097-1106 of ICP4 (LYPDAPPLRL; SEQ ID NO: 25), amino acids 170-179 of UL25 (SSGVVFGTWY; SEQ ID NO: 26), amino acids 235-243 of UL25 (AVLCLYLLY; SEQ ID NO: 27), amino acids 22-30 of UL26 (YVAGFLALY; SEQ ID NO: 28), amino acids 326-334 of UL26 (YLWIPASHY; SEQ ID NO: 29), amino acids 295-303 of UL27 (VYMSPFYGY; SEQ ID NO: 30), amino acids 641-649 of UL27 (FTFGGGYVY; SEQ ID NO: 31), amino acids 460-468 of UL29 (ALLAKMLFY; SEQ ID NO: 32), amino acids 895-903 of UL29 (YMANQILRY; SEQ ID NO: 33), amino acids 93-101 of UL46 (LASDPHYEY; SEQ ID NO: 34), amino acids 126-134 of UL46 (AILTQYWKY; SEQ ID NO: 35), amino acids 224-232 of UL46 (LLAYVSVLY; SEQ ID NO: 36), amino acids 333-341 of UL46 SIVHHHAQY (SEQ ID NO: 37), amino acids 508-516 of UL47 ALATVTLKY (SEQ ID NO: 38), amino acids 698-706 of ICP0 VPGWSRRTL (SEQ ID NO: 39), amino acids 382-390 of UL21 VPRPDDPVL (SEQ ID NO: 40), amino acids 281-290 of UL49 RPTERPRAPA (SEQ ID NO: 41), amino acids 70-78 of US1 APRIGGRRA (SEQ ID NO: 42), amino acids 22-30 of US7 VVRGPTVSL (SEQ ID NO: 43), amino acids 97-105 of US7 CPRRPAVAF (SEQ ID NO: 44), and amino acids 195-203 of US7 APASVYQPA (SEQ ID NO: 45).

2. The pharmaceutical composition of claim 1, wherein the polypeptide consists of one or more of the HSV-1 proteins selected from the group consisting of UL1, UL13, UL21, UL25, UL26, UL27, UL29, UL31, UL37, UL39, UL40, UL41, UL46, UL47, UL48, UL49, UL53, UL54, US1, US7, ICP0, and ICP4, optionally, up to 100 amino acid residues of linker sequence between said proteins.

3. The pharmaceutical composition of claim 1, wherein the polypeptide consists of one or more of the epitopes set forth in claim 1 and, optionally, up to 100 amino acid residues of linker sequence between said eptiopes.

4. The pharmaceutical composition of claim 1, wherein the polypeptide is a fusion protein comprising the isolated HSV polypeptide fused to a heterologous polypeptide.

5. The pharmaceutical composition of claim 4, wherein the fusion protein is soluble.

6. A vector comprising the polynucleotide of claim 1.

7. A host cell transformed with the vector of claim 6.

8. A method of producing a HSV-1 polypeptide comprising culturing the host cell of claim 7 and recovering the polypeptide so produced.

9. A HSV polypeptide produced by the method of claim 8.

10. A recombinant virus genetically modified to express a polypeptide recited in claim 1.

11. The recombinant virus of claim 10 which is an adenovirus or poxvirus.

12. A pharmaceutical composition comprising the virus of claim 10 and a pharmaceutically acceptable carrier, and optionally, an adjuvant.

13. A method of producing immune cells directed against HSV comprising contacting an immune cell with an antigen-presenting cell, wherein the antigen-presenting cell is modified to present an epitope included in amino acids as recited in claim 1.

14. The method of claim 13, wherein the T cell is a CD4+ or CD8+ T cell.

15. An immune cell produced by the method of claim 13.

16. A method of killing a HSV infected cell, of inhibiting HSV replication, of enhancing secretion of antiviral or immunomodulatory lymphokines, or of enhancing production of HSV-specific antibody, the method comprising contacting a HSV with the immune cell of claim 15.

17. A method of enhancing proliferation of HSV-specific T cells comprising contacting the HSV-specific T cells with an isolated polypeptide that comprises an epitope as recited in claim 1.

18. A method of inducing an immune response to an HSV infection in a subject, or of treating a HSV infection in a subject, comprising administering the composition of claim 1 to the subject.

19. A method of inducing an immune response to an HSV infection in a subject, or of treating a HSV infection in a subject, comprising administering the composition of claim 12 to the subject.

20. A method of treating a HSV infection in a subject comprising administering an antigen-presenting cell modified to present an epitope as recited in claim 1.
